(19) United States
(12) Patent Application Publication
(10) Pub. No.: US 2019/0002567 A1
(43) Pub. Date: Jan. 3, 2019

(54) NEW USE OF AN ANTI-CD303 TRANSMEMBRANE PROTEIN ANTIBODY

(71) Applicant: LABORATOIRE FRANCAIS DU FRACTIONNEMENT ET DES BIOTECHNOLOGIES, Les Ulis (FR)

(72) Inventors: Abdessatar Sami CHTOUROU, ELANCOURT (FR); Nathalie FOURNIER, Erquinghem-Lys (FR)

(21) Appl. No.: 16/062,284

(22) PCT Filed: Dec. 16, 2016
(86) PCT No.: PCT/FR2016/053504

§ 371 Date: Jun. 14, 2018

(30) Foreign Application Priority Data
Dec. 16, 2015 (FR) 1562545

(51) Int. Cl. C07K 16/28; A61P 35/00; A61K 47/68; A61K 39/395

(52) U.S. Cl. CPC C07K 16/2851; A61P 35/00; A61K 2039/58; A61K 39/39558; A61K 47/6803

(57) ABSTRACT

Disclosed is an anti-CD303 protein antibody for use in the prophylaxis or therapy of a tumor, involving plasmacytoid dendritic cell activation in the environment of the tumor, said plasmacytoid dendritic cells not being the cause of the tumor.

The present invention relates to a new use of an antibody directed against a membrane protein, and in particular for therapy in the treatment of relevant pathologies.

The use of anti-membrane protein antibodies directed against a membrane protein has already been disclosed in the prior art.

The European patent application EP 1 783 141 discloses for example a composition that makes it possible to induce an immune response in a patient, said composition comprising dendritic cells previously treated with an anti-CD303 antibody that expresses an antigen (tumour, viral, bacterial, etc). This consists of a cell therapy treatment.

The patent application WO 2006/037247 discloses the use of monoclonal antibodies directed against the CD303 protein, in the context of the treatment of a particular auto-immune disease, namely psoriasis.

Nestle et al. (Nestle et al. 2005, J. Exp. Med, vol 202, no 1, pp: 135-143) teaches that antibodies directed against the CD303 protein may be injected intravenously, for therapeutic purposes in the context of the treatment of psoriasis.

Blomberg et al. (Blomberg et al., 2003, Arthritis and Rheumatism, vol 48, no 9, pp: 2524-2532) teaches the use of antibodies directed against the protein CD303, in the context of an autoimmune disease: Lupus Erythematosus. The authors show that the said antibodies are capable of inhibiting the production of interferon α (IFN-α).

The patent application WO 01/36487 discloses monoclonal antibodies directed against the CD303 protein, and in particular the clones AC144, AD5-13A11 and ADS-4B8, as well as the fragments derived. Moreover, this application describes the use of monoclonal antibodies in the context of the treatment of pathologies such as viral infections, autoimmune diseases and tumours. However, this application does not disclose any specific example of treatment of a pathology or disease by making use of the indirect action of an antibody against the CD303 protein.

The international patent application WO 2012/080642 also discloses the use of antibodies directed against the CD303 protein in the context of prophylaxis or therapy to treat hematopoietic tumours of the CD4+/CD56+ phenotype, where plasmacytoid dendritic cells are believed to be the cause.

Plasmacytoid dendritic cells can be the cause of such hematopoietic tumours of the CD4+/CD56+ phenotype which are formed when they acquire an additional marker which is CD56+. This is why they are referred to as CD4+/CD56+ hematopoietic tumours. Such tumours are therefore the result of plasmacytoid dendritic cell-linked tumour development.

Plasmacytoid dendritic cells however are not the cause of all types of tumours.

There is therefore a real need to find a treatment therapy for tumours whereof the cause does not involve plasmacytoid dendritic cells, in particular tumours whereof the cause is not linked to the acquisition of an additional marker such as CD56+ by plasmacytoid dendritic cells.

This is why one of the goals of the invention is to provide a treatment therapy that makes it possible to prevent and/or treat tumours whereof the cause does not involve plasmacytoid dendritic cells, in particular tumours whereof the cause is not linked to the acquisition of an additional marker by the plasmacytoid dendritic cells.

This is why one of the goals of the invention is to prevent and/or treat tumours not resulting from plasmacytoid dendritic cell-linked tumour development.

The present invention thus relates to a new use of an antibody directed against a membrane protein for the prevention and/or treatment of tumours whereof the cause does not involve plasmacytoid dendritic cells.

The present invention also relates to a novel composition comprising a mixture of anti-membrane protein antibodies directed against a membrane protein, and use thereof for the prevention and/or treatment of tumours whereof the cause does not involve plasmacytoid dendritic cells.

The present invention also relates to a new use of an anti-cancer agent.

The present invention is based on the unexpected finding of the inventors which is that the anti-membrane protein antibodies directed against a membrane protein, in particular the CD303 protein, can be used for the prevention and/or treatment of tumours whereof the cause does not involve the acquisition of a marker by the plasmacytoid dendritic cells, in other words for the prevention and/or treatment of tumours not resulting from plasmacytoid dendritic cell-linked tumour development.

The invention is thus distinctly differentiated from the prior art in that it removes the plasmacytoid dendritic cells by the cytotoxic action of the antibody directed against the CD303 protein, in order to prevent and/or treat tumours, although these plasmacytoid dendritic cells are not responsible for causing the tumour. Indeed, although they are not the cause of the tumour, the plasmacytoid dendritic cells, however, can promote the growth of tumour cells and survival thereof, for example by developing immunosuppressive and/or tolerogenic properties. This can thus inhibit or reduce the effectiveness of anticancer medicaments/agents conventionally used in monotherapy. Thus, in the case of the present invention, antibodies directed against the protein CD303 have an indirect action on the treatment of tumours: antibodies directed against the protein CD303 make it possible to decrease the plasmacytoid dendritic cells in the tumour microenvironment, and thereby decrease, advantageously eliminate the immunosuppressive and/or tolerogenic properties of the latter in the tumour microenvironment. According to the invention, the antibodies directed against the CD303 protein can thus facilitate, stimulate or potentiate the action of the anti-cancer agents (for example, an anti-tumour antigen antibody) with which they may be used in a simultaneous, separate or sequential manner over time. The antibodies directed against the CD303 protein according to the invention may also have a synergy with the anti-cancer agents with which it may be used.

In a first aspect, the invention thus relates to an antibody directed against CD303 protein for use in the prevention and/or treatment of a tumour involving activation of plasmacytoid dendritic cells in the microenvironment of the said tumour, the said plasmacytoid dendritic cells not being responsible for causing the tumour.

The term “antibody” is used to refer to an immunoglobulin, a protein constituted of four chains participating in the acquired immune response or an immunoglobulin fragment. Immunoglobulins are well known to the person skilled in the art and consist of an assembly of two dimers each constituted of a heavy chain and a light chain. The multimeric complex is assembled by the binding of a light chain and a heavy chain by a disulfide bond between two cysteines, the two heavy chains themselves being also connected to each other by two disulfide bonds.

Each of the heavy chains and light chains is constituted of a constant region and a variable region. The assembly of the chains that make up an antibody make it possible to define a characteristic three-dimensional structure that is Y-shaped, wherein
the base of the Y corresponds to the Fc constant region that is recognized by the complement and the Fc receptors, and
the end of the arms of the Y corresponds to the respective assembly of the variable regions of the light chain and the heavy chain which are recognized by a specific antigen.

More precisely, each light chain is constituted of a variable region (V_{L}) and a constant region (C_{L}). Each heavy chain is constituted of a variable region (V_{H}) and a constant region constituted of three constant domains C_{H1}, C_{H2} and C_{H3}. The Fc domain comprises of the C_{H2} and C_{H3} domains.

The variable regions of the light chain and the heavy chain are constituted of three domains determining the recognition of the antigen (CDR regions for Complementary Determining Regions) surrounded by four framework domains (FR for Framework Regions). The three-dimensional folding of the variable region is such that the three CDRs are exposed on the same side of the protein and enable the formation of a specific structure recognizing a particular determined antigen.

The antibodies described herein are isolated and purified, and are different from natural antibodies. These antibodies are mature, that is to say, they have an ad hoc three-dimensional structure that allows them to recognize the antigen, and have all of the post-translational modifications essential to their antigen recognition.

According to the invention an “antibody directed against the CD303 protein” is understood to refer to an “anti-CD303” antibody.

The term “CD303 protein” is understood to refer to the protein formerly known as BDCA-protein 2. This protein is expressed in a specific manner on the surface of the plasmacytoid dendritic cells, and is a type II protein belonging to the C-type lectins.

In other words, the human CD303 antigen (or CD303 protein) is the C-type lectin domain family 4 member C and is also known as CLEC4 (for “C-type lectin domain family 4, member C”); DLEC; HECL; BDCA-2; CLECSF7; CLECSF11; or PRO34150 (see the EntrezGene site for the gene CLEC4). It is a type II transmembrane glycoprotein of 213 amino acids, comprising a short cytoplasmic domain that lacks any obvious signaling motifs (amino acids 1-21), a transmembrane region (amino acids 22-41), a neck domain (amino acids 42-82), and an extracellular carbohydrate recognition domain (CRD for “carbohydrate recognition domain”; amino acids 83-213) (Dzionek et al—2001). The sequence of the mRNA encoding for this protein may be found in the Genbank database in the version thereof dated 14 Feb. 2002 under accession no AF293615.1 (SEQ ID NO: 129), while the sequence of amino acids is accessible through the Genbank database in the version thereof dated 14 Feb. 2002 under accession no AAL37036.1 (SEQ ID NO: 130).

The term “plasmacytoid dendritic cells” is understood to refer to the subpopulation of dendritic cells also known as DC2. The plasmacytoid dendritic cells are characterised by the lineage (Lin) markers (CD3−, CD19−, CD20−, CD14−, CD56−), HLA-DR+, CD11c−, CD123+, and CD45RA+. These cells have also been characterised phenotypically: they express the CD4 and CD303, and BDCA-4 markers. They are present in the lymphoid organs and are also in circulation in the blood. The plasmacytoid dendritic cells have the capacity to secrete IFN type I in the presence of a viral infection.

The term “activation of plasmacytoid dendritic cells in the microenvironment of the said tumour” is understood to refer to the different mechanisms that have the effect of activating the proliferation, and secretion of specific determined cytokines, in particular class I interferons, and the phenotypic and morphological alteration of plasmacytoid cells.

The plasmacytoid dendritic cells may also promote tumour cell growth and survival, in particular by inducing an immunosuppressive environment within the tumour microenvironment, for example by inducing the differentiation of regulatory T lymphocyte cells (Treg). Thus, the term “activation of plasmacytoid dendritic cells in the microenvironment of the said tumour” is also understood to refer to the immunosuppressive and/or tolerogenic properties that the plasmacytoid dendritic cells can have vis-a-vis the tumour.

The term “the said plasmacytoid dendritic cells not being responsible for causing the tumour” signifies that the tumours according to the present invention are not linked to tumour development involving plasmacytoid dendritic cells. More precisely, the tumours according to the present invention are not linked to the acquisition by plasmacytoid dendritic cells of an additional marker such as CD56.

The term “prevention” is to be understood as the prevention of tumour expansion in situ, or indeed as the prevention of development of metastasis from tumours already infiltrated in their microenvironment by plasmacytoid dendritic cells.

In one particular aspect of the invention, the plasmacytoid dendritic cells have immunosuppressive and/or tolerogenic properties.

The term “immunosuppressive properties” is understood to refer to the properties of dendritic cells to develop and maintain immunosuppression in the microenvironment of the tumour.

The term “tolerogenic properties” signifies that plasmacytoid dendritic cells will not induce an immune response.

In one particular aspect of the invention, the tumours involving activation of plasmacytoid dendritic cells are solid tumour or hematopoietic tumours.

The term “solid tumours” is understood to refer to a cellular mass resulting from an excessive multiplication of cells. Solid tumours may develop in any tissue, in particular the skin, mucous membranes, the bones or organs, etc. Solid tumours may be distinguished into two groups: carcinomas and sarcomas.

Carcinomas are derived from epithelial cells (present in the skin, mucous membranes, or glands); the cancers involved are for example breast cancer, lung cancer, prostate cancer, intestinal cancer, etc.

Sarcomas are derived from connective tissue cells; the cancers involved are for example of the bone cancer, cartilage cancer, etc.

The term “hematopoietic tumours” refers to tumours involving cells of the blood line, or hematopoietic cells, or tumours affecting the hematopoietic organs, namely the organs capable of hematopoiesis that participate in the development of blood cells. Hematopoietic organs are the bone marrow and those forming the lymphoid tissue (the thymus, ganglia, and spleen, for example). Hematopoietic tumours may also be known as hematological malignancies.

In one particular aspect of the invention, solid tumours involve an infiltration of plasmacytoid dendritic cells in the microenvironment of the said tumour, and preferably belong to the group of tumours of the head and neck, melanoma, urogenital cancers, breast cancer.

The term “solid tumours involve an infiltration by plasmacytoid dendritic cells in the microenvironment of the said tumour” signifies that plasmacytoid dendritic cells are recruited to the tumour site. The plasmacytoid dendritic cells recruited to the tumour site have particular immunosuppressive and/or tolerogenic properties. The plasmacytoid dendritic cells infiltrated into the tumour microenvironment can also induce the differentiation of regulatory T lymphocyte cells and/or induce the infiltration of these latter into the tumour site.

The term “tumours of the head and neck” is understood to refer to cancers of the oral cavity, pharynx, nasopharynx, larynx, nasal cavity, sinuses, or salivary glands.

The term “melanoma” is understood to refer to a malignant tumour that develops from skin cells known as melanocytes. Four main types of skin melanoma exist: superficial spreading melanoma, nodular melanoma, Dubreuilh melanoma or lentigo maligna melanoma and acral lentiginous melanoma.

The term “urogenital cancers” is understood to refer to cancer of the urogenital tract in male or female organs. The cancers involved are for example prostate cancer, testicular cancer, penile cancer, endometrial cancer, cancer of the vulva and vagina, cancer of the uterus, cervical cancer, ovarian cancer, kidney cancer, bladder cancer.

The term “breast cancer” is understood to refer to cancer of the mammary glands, whether it be non-invasive (Ductal Carcinoma In Situ (DCIS)/Intraductal Carcinoma In Situ) or invasive.

In one particular aspect of the invention, the hematopoietic tumours belong to the group consisting of multiple myeloma, lymphoma, leukemia, in particular T cell leukemia.

The term “multiple myeloma” is understood to refer to a bone marrow disease/disorder characterised by the multiplication in the bone marrow of an abnormal plasma cell.

The term “lymphoma” is understood to refer to tumours in which blood cells proliferate abnormally in secondary lymphoid organs (lymph nodes, spleen, etc).

The term “leukemia” is understood to refer to tumours in which blood cells proliferate abnormally in the blood.

In one particular aspect of the invention, the said antibody for the aforementioned use thereof is monoclonal or polyclonal.

The term “monoclonal” is understood to refer to an antibody that recognizes only one unique epitope in CD303, unlike polyclonal antibodies which correspond to a mixture of monoclonal antibodies, and therefore can recognize multiple epitopes on the same given protein.

The term “monoclonal antibodies” or “monoclonal antibody composition” is understood to refer to a composition comprising antibody molecules having an identical and unique antigen specificity. The antibody molecules present in the composition are likely to differ in terms of their post-translational modifications, and in particular with respect to their glycosylation structures or their isoelectric point, but have all been encoded by the same sequences of heavy and light chains and therefore, prior to any post-translational modification, have the same protein sequence. Certain differences in the protein sequences linked to post-translational modifications (for example, cleavage of the heavy chain C-terminal lysine, deamidation of asparagine residues and/or isomerisation of aspartate residues) may nevertheless exist between the various different antibody molecules present in the composition.

The monoclonal antibodies of the invention may be obtained by techniques well known to the person skilled in the art, in particular the cell fusion technique, the technique of cloning sequences of heavy and light chains, the technique of phage or ribosome display by immunisation of mice having the human immunoglobulin repertoire and expression in an ad hoc cell or in a transgenic animal.

In one particular aspect of the invention, the said antibody for the aforementioned use thereof is selected from among a murine antibody, a chimeric antibody, a humanised antibody or a human antibody.

In one particular aspect of the invention, the said antibody for the aforementioned use thereof is a chimeric antibody, and preferably a chimeric antibody selected from a murine/human chimeric antibody or a human-macaque chimeric antibody.

In one particular aspect of the invention, the antibody, functional fragment or derivative thereof according to the invention is advantageously a chimeric or humanised antibody, particularly a chimeric antibody in which the constant region of the heavy and light chains is of human origin.

The term “murine antibody” is understood to refer to an antibody wherein the constituent sequences of heavy chains and light chains are sequences whose nucleic acid correspondence is found in the genome of murine B cells. This antibody is thus constituted of murine amino acid sequences, whatever the origin of the cell which enables the production thereof. For example, mouse antibody sequences expressed in macaque monkey cells will provide murine antibodies.

The above definition applies mutatis mutandis to human antibodies.

The term “chimeric antibody” is understood to refer to an isolated antibody, wherein each light chain and/or each heavy chain sequence of which it is constituted comprises or consists of a hybrid sequence derived from at least two different animals. In particular, the chimeric antibodies of the invention are human/macaque monkey or human/mouse hybrids, which signifies that one region of the sequence of light chains and heavy chains is derived from the sequence of a macaque or mouse immunoglobulin, and that the rest of the sequence of the said heavy chains and the said light chains is derived from the sequence of one, or possibly more, human immunoglobulins. The term “chimeric antibody” is also understood to refer to an antibody that contains a variable region (light chain and heavy chain) naturally derived from an antibody of a given species in combination with the constant regions of light chain and heavy chain of an antibody of a species heterologous to the said given species. Advantageously, where the monoclonal antibody composition for use thereof as a medicinal product according to the invention comprises a chimeric monoclonal antibody, it comprises human constant regions. Starting from a non-human antibody, a chimeric antibody may be prepared using genetic recombination techniques well known to the person skilled in the art. For example, the chimeric antibody may be prepared by cloning for the heavy chain and the light chain, a recombinant DNA comprising a promoter and a sequence encoding for the variable region of the nonhuman antibody, and a sequence encoding for the constant region of a human antibody. For methods for preparing chimeric antibodies, one could for example refer to the document Verhoeyn et al, 1988.

The term “humanised antibody” is understood to refer to an antibody derived from a non-human animal in which the sequences of heavy chains and light chains other than CDRs have been replaced by corresponding sequences of one or more antibodies of human origin. The antibody is therefore predominantly constituted of human sequences, but its specificity for the antigen provided by the CDRs is derived from another species. The term “humanised antibody” is also understood to refer to an antibody which contains CDR regions derived from a non-human antibody, the other parts of the antibody molecule being derived from one (or more) human antibodies. In addition, some of the residues of the framework segments (referred to as FR) may be modified so as to retain the binding affinity (Jones et al—1986; Verhoeyen et al 1988; Riechmann et al—1988). The humanised antibodies according to the invention may be prepared by techniques known to the person skilled in the art, such as the following technologies: “CDR grafting”, “resurfacing”, SuperHumanisation, “Human string content”, “FR libraries”, “Guided selection”, “FR shuffling”, and “Humaneering”, as summarised in the review article by Almagro et al—2008.

In one particular aspect of the invention, the said antibody for the aforementioned use thereof is a monoclonal antibody directed against the ectodomain of the human CD303 antigen (SEQ ID NO: 130), or a functional fragment or derivative thereof, characterised in that:
a) it competes for binding to the human CD303 antigen with at least one antibody selected from among:
   i) An antibody whose heavy chain variable region includes the sequence SEQ ID NO: 43 and the light chain variable region includes the sequence SEQ ID NO: 48;
   ii) An antibody whose heavy chain variable region includes the sequence SEQ ID NO: 44 and the light chain variable region includes the sequence SEQ ID NO: 49;
   iii) An antibody whose heavy chain variable region includes the sequence SEQ ID NO: 45 and the light chain variable region includes the sequence SEQ ID NO: 50;
   iv) An antibody whose heavy chain variable region includes the sequence SEQ ID NO: 46 and the light chain variable region includes the sequence SEQ ID NO: 51;
   v) An antibody whose heavy chain variable region includes the sequence SEQ ID NO: 47 and the light chain variable region includes the sequence SEQ ID NO: 52; and
b) the constant regions of the light chains and heavy chains are constant regions derived from a non-murine species.

Advantageously, the heavy chains comprise three CDR-Hs (heavy chain CDR according to IMGT nomenclature) having the following amino acid sequences, or sequences having at least 80% identity with the following sequences, and the light chains comprise three CDR-Ls (light chain CDR according to IMGT nomenclature) having the following amino acid sequences, or sequences having at least 80% identity with the following sequences:

i) CDR1-H-family 1: SEQ ID NO: 1, CDR2-H-family 1: SEQ ID NO: 2, CDR3-H-family 1: SEQ ID NO: 3, CDR1-L-family 1: SEQ ID NO: 4, CDR2-L-family 1: SEQ ID NO: 5, CDR3-L-family 1: SEQ ID NO: 6; or

ii) CDR1-H-family 2: SEQ ID NO: 7, CDR2-H-family 2: SEQ ID NO: 8, CDR3-H-family 2: SEQ ID NO: 9, CDR1-L-family 2: SEQ ID NO: 10, CDR2-L-family 2: SEQ ID NO: 11, CDR3-L-family 2: SEQ ID NO: 12.

The term “at least 80% identity” signifies a degree of identity of 80%, 81%, 82%, 83%, 84%, 85%, 86%, 87%, 88%, 89%, 90%, 91%, 92%, 93%, 94%, 95%, 96%, 97%, 98%, 99% and 100%.

Table 1 below summarises the amino acid sequences of the CDRs-IMGT of the two families of antibodies that may be used according to the invention:

TABLE 1
Amino acid sequences of the CDRs of the two
families of antibodies that may be used according
to the invention according to IMGT nomenclature.
In each sequence, X may represent any amino acid.

| | Family 1 | Family 2 |
|---|---|---|
| CDR1-H | GYTFTDYS (SEQ ID NO: 1) | GYTFTDXS |
| | | (SEQ ID NO: 7) |
| CDR2-H | ISXYYGDX (SEQ ID NO: 2) | INTETGXP |
| | | (SEQ ID NO: 8) |
| CDR3-H | ARNXXXYXXXY (SEQ ID NO: | XRNGYYVGYYAXDY |
| | 3) | (SEQ ID NO: 9) |
| CDR1-L | QDIXNY (SEQ ID NO: 4) | SSVXY |
| | | (SEQ ID NO: 10) |
| CDR2-L | YTS (SEQ ID NO: 5) | STS (SEQ ID NO: 11) |
| CDR3-L | QQGXTLPWT (SEQ ID NO: 6) | QQRRSYPXT (SEQ ID |
| | | NO: 12) |

Advantageously, the heavy chains of an antibody, functional fragment or derivative thereof according to the invention comprise three CDR-Hs (heavy chain CDR according to IMGT nomenclature) having the following amino acid sequences, or sequences having at least 80% identity with the following sequences, and the light chain comprises three CDR-Ls (light chain CDR according to IMGT nomenclature) having the following amino acid sequences, or sequences having at least 80% identity with the following sequences:
i) CDR1-H-122A2: SEQ ID NO: 13, CDR2-H-122A2: SEQ ID NO: 14, CDR3-H-122A2: SEQ ID NO: 15, CDR1-L-122A2: SEQ ID NO: 16, CDR2-L-122A2: SEQ ID NO: 17, CDR3-L-122A2: SEQ ID NO: 18;
ii) CDR1-H-102E9: SEQ ID NO: 19, CDR2-H-102E9: SEQ ID NO: 20, CDR3-H-102E9: SEQ ID NO: 21, CDR1-L-102E9: SEQ ID NO: 22, CDR2 L-102E9: SEQ ID NO: 23, CDR3-L-102E9: SEQ ID NO: 24;
iii) CDR1-H-104C12: SEQ ID NO: 25, CDR2-H-104C12: SEQ ID NO: 26, CDR3-H-104C12: SEQ ID NO: 27, CDR1-L-104C12: SEQ ID NO: 28, CDR2 L-104C12: SEQ ID NO: 29, CDR3-L-104C12: SEQ ID NO: 30;
iv) CDR1-H-114D11: SEQ ID NO: 31, CDR2-H-114D11: SEQ ID NO: 32, CDR3-H-114D11: SEQ ID NO: 33, CDR1-L-114D11: SEQ ID NO: 34, CDR2 L-114D11: SEQ ID NO: 35, CDR3-L-114D11: SEQ ID NO: 36; or
v) CDR1-H-104E10: SEQ ID NO: 37, CDR2-H-104E10: SEQ ID NO: 38, CDR3-H-104E10: SEQ ID NO: 39, CDR1-L-104E10: SEQ ID NO: 40, CDR2-L-104E10: SEQ ID NO: 41, CDR3-L-104E10: SEQ ID NO: 42.

Advantageously, the heavy chains of an antibody, functional fragment or derivative thereof according to the invention comprise a variable region having a sequence selected from SEQ ID NO: 43 to 47 or a sequence having at least 80% identity with one of SEQ ID NO: 43 to 47.

Additionally or alternatively, the light chains of an antibody, functional fragment or derivative thereof according to the invention comprise a variable region having a sequence selected from SEQ ID NO: 48 to 52 or a sequence having at least 80% identity with one of SEQ ID NO: 48 to 52.

In one preferred embodiment, the antibody, functional fragment or derivative thereof according to the invention has heavy and light chains whose variable regions have the following amino acid sequences or sequences having at least 80% identity with the following sequences:
i) Antibody 122A2: heavy chain: SEQ ID NO: 43, light chain: SEQ ID NO: 48,
ii) Antibody 102E9: heavy chain: SEQ ID NO: 44, light chain: SEQ ID NO: 49,
iii) Antibody 104C12: heavy chain: SEQ ID NO: 45, light chain: SEQ ID NO: 50,
iv) Antibody 114D11: heavy chain: SEQ ID NO: 46, light chain: SEQ ID NO: 51, or
v) Antibody 104E10: heavy chain: SEQ ID NO: 47, light chain: SEQ ID NO: 52.

Table 2 here below summarises the murine VH, JH, and VL and JL gene segments used by the different antibodies according to the invention and the percentage of identity.

TABLE 2
Murine VH, JH, and VL and JL segments used by the different
antibodies according to the invention, as defined by IMGT.

| Antibody | VH | JH | VL | JL |
|---|---|---|---|---|
| 122A2 | IGHV1S137*01 | IGHJ2*02 | IGKV10-96*01 | IGKJ1*01 |
| | (94.9%) | (85%) | (98.9%) | (100%) |
| 102E9 | IGHV9-2-1*01 | IGHJ4*01 | IGKV4-57*01 | IGKJ1*02 |
| | (93.9%) | (100%) | (95.7%) | (100%) |
| 104C12 | IGHV1S137*01 | IGHJ3*01 | IGKV10-96*02 | IGKJ1*02 |
| | (91.8%) | (100%) | (89.5%) | (100%) |
| 114D11 | IGHV9-2-1*01 | IGHJ4*01 | IGKV4-57*01 | IGKJ1*02 |
| | (94.9%) | (94.1%) | (97.9%) | (100%) |
| 104E10 | IGHV9-2-1*01 | IGHJ4*01 | IGKV4-57*01 | IGKJ1*02 |
| | (98%) | (100%) | (96.8%) | (100%) |

Table 3 here below summarises the amino acid sequences of the CDRs and the variable regions of heavy and light chains of the anti-CD303 antibodies generated by the inventors, according to the invention:

TABLE 3
Amino acid sequences of heavy- and light chain CDR1, CDR2,
and CDR3 according to IMGT nomenclature, and of the VH and
VL fragments of the antibodies according to the invention.
Antibody 122A2
Heavy Chain

| | |
|---|---|
| CDR1-H- | GYTFTDYS (SEQ ID NO: 13) |
| IMGT- | |
| 122A2 | |
| CDR2-H- | ISTYYGDS (SEQ ID NO: 14) |
| IMGT- | |
| 122A2 | |
| CDR3-H- | ARNGNFYVMDY (SEQ ID NO: 15) |
| IMGT- | |
| 122A2 | |
| VH-122A2 | QVQLQQSGAELVRPGVSVKISCKGSGYTFTDYSMHWVKQSHAKSLEW |
| | IGVISTYYGDSNYNQKFKGKATMTVDKSSTTAYMELARLTSEDSAIYYC |
| | ARNGNFYVMDYWGQGTSVTVSS (SEQ ID NO: 43) |

Light Chain

| | |
|---|---|
| CDR1-L- | QDISNY (SEQ ID NO: 16) |
| IMGT- | |
| 122A2 | |
| CDR2-L- | YTS (SEQ ID NO: 17) |
| IMGT- | |
| 122A2 | |
| CDR3-L- | QQGNTLPWT (SEQ ID NO: 18) |
| IMGT- | |
| 122A2 | |
| V-122A2 | DIQMTQTTSSLSASLGDRVTISCRASQDISNYLNWYQQKPDGTVKLLIY |
| | YTSRLHSGVPSRFSGSGSGTDYSLTISNLDQEDIATYFCQQGNTLPWTF |
| | GGGTKLEIK (SEQ ID NO: 48) |

Antibody 102E9
Heavy Chain

| | |
|---|---|
| CDR1-H- | GYTFTDYS (SEQ ID NO: 19) |
| IMGT- | |
| 102E9 | |
| CDR2-H- | INTETGEP (SEQ ID NO: 20) |
| IMGT- | |
| 102E9 | |
| CDR3-H- | TRNGYYVGYYAMDY (SEQ ID NO: 21) |
| IMGT- | |
| 102E9 | |
| VH- | QIHLVQSGPDLKKPGETVKISCKASGYTFTDYSMHWVKQAPGKGLKW |
| 102E9 | MGWINTETGEPTYADDFKGRFAFSLESSASTAFLQINNLKNEDTSTYFC |
| | TRNGYYVGYYAMDYWGQGTSVTVSS (SEQ ID NO: 44) |

Light Chain

| | |
|---|---|
| CDR1-L- | SSVIY (SEQ ID NO: 22) |
| IMGT- | |
| 102E9 | |
| CDR2-L- | STS (SEQ ID NO: 23) |
| IMGT- | |
| 102E9 | |
| CDR3-L- | QQRRSYPFT (SEQ ID NO: 24) |
| IMGT- | |
| 102E9 | |
| V-102E9 | QIVLTQSPAIMSASPGEKVTITCSASSSVIYIHWFQQKPGTSPKLWIYST |
| | SYLASGVPARFSGSGSGTSYSLTISRMEAEDAATYYCQQRRSYPFTFG |
| | GGTKLEIK (SEQ ID NO: 49) |

Antibody 104C12
Heavy Chain

| | |
|---|---|
| CDR1-H- | GYTFTDYS (SEQ ID NO: 25) |
| IMGT- | |
| 104C12 | |
| CDR2-H- | ISPYYGDT (SEQ ID NO: 26) |
| IMGT- | |
| 104C12 | |
| CDR3-H- | ARNDDYYRFAY (SEQ ID NO: 27) |
| IMGT- | |
| 104C12 | |
| VH-104C12 | QVQLQQSGAELVGPGVSVKISCKGSGYTFTDYSMHWVKQSHAKSLEW |
| | IGVISPYYGDTNYNQKFKGKATMTVDKSSSTAYMELASLTSEDSAIYFC |
| | ARNDDYYRFAYWGQGTLVTVSA (SEQ ID NO: 45) |

Light Chain

| | |
|---|---|
| CDR1-L- | QDINNY (SEQ ID NO: 28) |
| IMGT- | |
| 104C12 | |
| CDR2-L- | YTS (SEQ ID NO: 29) |
| IMGT- | |
| 104C12 | |
| CDR3-L- | QQGKTLPWT (SEQ ID NO: 30) |
| IMGT- | |
| 104C12 | |
| VL- | DLQMTQTPSSLSASLGDRVTISCRASQDINNYLSWYQEKPDGTFKLLIY |
| 104C12 | YTSRLHSGVPSRFSGSGSGTDYSLTVRNLEQEDIGTYFCQQGKTLPWT |
| | FGGGTKLEIR (SEQ ID NO: 50) |

Antibody 114D11
Heavy Chain

| | |
|---|---|
| CDR1-H- | GYTFTDSS (SEQ ID NO: 31) |
| IMGT- | |
| 114D11 | |
| CDR2-H- | INTETGGP (SEQ ID NO: 32) |
| IMGT- | |
| 114D11 | |
| CDR3-H- | ARNGYYVGYYALDY (SEQ ID NO: 33) |
| IMGT- | |
| 114D11 | |
| VH- | QIQLVQSGPELKKPGETVKISCKASGYTFTDSSMHWVQQAPNKGLKW |
| 114D11 | MGWINTETGGPTYADDFKGRFAFSLETSARTAYLQINNLKNEDTATYFC |
| | ARNGYYVGYYALDYWGQGTSVTVSS (SEQ ID NO: 46) |

Light Chain

| | |
|---|---|
| CDR1-L- | SSVFY (SEQ ID NO: 34) |
| IMGT- | |
| 114D11 | |
| CDR2-L- | STS (SEQ ID NO: 35) |
| IMGT- | |
| 114D11 | |
| CDR3-L- | QQRRSYPYT (SEQ ID NO: 36) |
| IMGT- | |
| 114D11 | |
| VL- | QIVLTQSPAIMSASPGEKVTITCSASSSVFYMHWFQQKPGTSPKLWIYS |
| 114D11 | TSNLASGVPARFSGSGSGTSYSLTISRMEAEDAATYYCQQRRSYPYTF |
| | GGGTKLEIK (SEQ ID NO: 51) |

Antibody 104E10
Heavy Chain

| | |
|---|---|
| CDR1-H- | GYTFTDYS (SEQ ID NO: 37) |
| IMGT- | |
| 104E10 | |
| CDR2-H- | INTETGEP (SEQ ID NO: 38) |
| IMGT- | |
| 104E10 | |
| CDR3-H- | ARNGYYVGYYAMDY (SEQ ID NO: 39) |
| IMGT- | |
| 104E10 | |
| VH- | QIQLVQSGPELKKPGETVKISCKASGYTFTDYSMHWVKQAPGKGLKW |
| 104E10 | MGWINTETGEPTYADDFKGRFAFSLETSATTAYLQINNFKNEDTATYFC |
| | ARNGYYVGYYAMDYWGQGTSVTVSS (SEQ ID NO: 47) |

Light Chain

| | |
|---|---|
| CDR1-L- | SSVIY (SEQ ID NO: 40) |
| IMGT- | |
| 104E10 | |
| CDR2-L- | STS (SEQ ID NO: 41) |
| IMGT- | |
| 104E10 | |
| CDR3-L- | QQRRSYPYT (SEQ ID NO: 42) |
| IMGT- | |
| 104E10 | |
| VL- | QIVLTQSPAIMSASPGEKVTMTCSASSSVIYMHWFQQKPGTSPKLWIYS |
| 104E10 | TSNLASGVPARFSGSGSGTSYSLTISRMEAEDAATYYCQQRRSYPYTF |
| | GGGTKLEIK (SEQ ID NO: 52) |

In one particular aspect of the invention, the said chimeric, humanised or human antibody, functional fragment or derivative thereof, for the abovementioned use thereof, is an antibody whose heavy chain variable region is represented by the sequence SEQ ID NO: 43 or a sequence having at least 80% identity with the said SEQ ID NO: 43, and whose light chain variable region is represented by the sequence SEQ ID NO: 48 or a sequence having at least 80% identity with the said SEQ ID NO: 48 (antibody 122A2).

In one particular aspect of the invention, the said chimeric, humanised or human antibody, functional fragment or derivative thereof, for the abovementioned use thereof, is an antibody whose heavy chain variable region is represented by the sequence SEQ ID NO: 44 or a sequence having at least 80% identity with the said SEQ ID NO: 44, and whose light chain variable region is represented by the sequence SEQ ID NO: 49, or a sequence having at least 80% identity with the said SEQ ID NO: 49 (antibody 102E9).

In one particular aspect of the invention, the said chimeric, humanised or human antibody, functional fragment or derivative thereof, for the abovementioned use thereof, is an antibody whose heavy chain variable region is represented by the sequence SEQ ID NO: 45, or a sequence having at least 80% identity with the said SEQ ID NO: 45, and whose light chain variable region is represented by the sequence SEQ ID NO: 50 or a sequence having at least 80% identity with the said SEQ ID NO: 50 (antibody 104C12).

In one particular aspect of the invention, the said chimeric, humanised or human antibody, functional fragment or derivative thereof, for the abovementioned use thereof, is an antibody whose heavy chain variable region is represented by the sequence SEQ ID NO: 46, or a sequence having at least 80% identity with the said SEQ ID NO: 46, and whose light chain variable region is represented by the sequence SEQ ID NO: 51, or a sequence having at least 80% identity with the said SEQ ID NO: 51 (antibody 114D11).

In one particular aspect of the invention, the said chimeric, humanised or human antibody, functional fragment or derivative thereof, for the abovementioned use thereof, is an antibody whose heavy chain variable region is represented by the sequence SEQ ID NO: 47, or a sequence having at least 80% identity with the said SEQ ID NO: 47, and whose light chain variable region is represented by the sequence SEQ ID NO: 52, or a sequence having at least 80% identity with the said SEQ ID NO: 52 (antibody 104E10).

In one particular aspect of the invention, the said chimeric, humanised or human antibody, functional fragment or derivative thereof, for the abovementioned use thereof, is an antibody whose heavy chain is represented by the sequence SEQ ID NO: 55, or a sequence having at least 80% identity with the said SEQ ID NO: 55, and whose light chain is represented by the sequence SEQ ID NO: 60, or a sequence having at least 80% identity with the said SEQ ID NO: 60 (antibody 122A2).

In one particular aspect of the invention, the said chimeric, humanised or human antibody, functional fragment or derivative thereof, for the abovementioned use thereof, is an antibody whose heavy chain is represented by the sequence SEQ ID NO: 56, or a sequence having at least 80% identity with the said SEQ ID NO: 56, and whose light chain is represented by the sequence SEQ ID NO: 61, or a sequence having at least 80% identity with the said SEQ ID NO: 61 (antibody 102E9).

In one particular aspect of the invention, the said chimeric, humanised or human antibody, functional fragment or derivative thereof, for the abovementioned use thereof, is an antibody whose heavy chain is represented by the sequence SEQ ID NO: 57, or a sequence having at least 80% identity with the said SEQ ID NO: 57, and whose light chain is represented by the sequence SEQ ID NO: 62, or a sequence having at least 80% identity with the said SEQ ID NO: 62 (antibody 104C12).

In one particular aspect of the invention, the said chimeric, humanised or human antibody, functional fragment or derivative thereof, for the abovementioned use thereof, is an antibody whose heavy chain is represented by the sequence SEQ ID NO: 58, or a sequence having at least 80% identity with the said SEQ ID NO: 58, and whose light chain is represented by the sequence SEQ ID NO: 63, or a sequence having at least 80% identity with the said SEQ ID NO: 63 (antibody 114D11).

In one particular aspect of the invention, the said chimeric, humanised or human antibody, functional fragment or derivative thereof, for the abovementioned use thereof, is an antibody whose heavy chain is represented by the sequence SEQ ID NO: 59, or a sequence having at least 80% identity with the said SEQ ID NO: 59, and whose light chain is represented by the sequence SEQ ID NO: 64, or a sequence having at least 80% identity with the said SEQ ID NO: 64 (antibody 104E10).

The identity percentages to which reference is made in the context of the disclosure of the present invention are determined on the basis of a global alignment of the sequences to be compared, that is to say on an alignment of sequences taken into consideration in their entirety over the entire length by using any suitable algorithm well-known to the person skilled in the art such as the Needleman and Wunsch algorithm, 1970. This sequence comparison may be carried out, using any suitable software well-known to the person skilled in the art, for example the software application needle using the parameter “Gap open” equal to 10.0, the parameter “Gap extend” equal to 0.5, and a “BLOSUM 62” matrix. The software application needle is for example available on the website ebi.ac.uk under the name “Align”.

When the CDR or variable region of an antibody that may be used according to the invention has an amino acid sequence which is not 100% identical to one of those described here above and in the Sequence Listing (reference sequences) but which has at least 80%, preferably at least 85%, at least 90%, at least 95%, at least 96%, at least 97%, at least 98%, at least 99% identity with one such reference sequence, it may have insertions, deletions or substitutions with respect to the reference sequence. In the case of substitutions, the substitution is preferably made by an “equivalent” amino acid, that is to say any amino acid whose structure is similar to that of the original amino acid and therefore has little to no likelihood of modifying the biological activity of the antibody. Examples of such substitutions are presented in the following Table 4:

TABLE 4
Substitutions with equivalent amino acids

| Original amino acid | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (G) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

The antibodies may be of several isotypes, according to the nature of their constant region: the γ, α, μ, ε and δ constant regions correspond to immunoglobulins IgG, IgA, IgM, IgE and IgD, respectively. Advantageously, the monoclonal antibody present in a composition used as a medicinal product as such in the context of the invention is of isotype IgG. Indeed, this isotype shows a capacity to generate “Antibody-Dependent Cellular Cytotoxicity” (ADCC) activity in the greatest number of individuals (humans). The γ constant regions comprise several subtypes: γ1, γ2, γ3, these three types of constant regions having the feature of binding human complement, and γ4, thus creating the sub-isotypes IgG1, lgG2, IgG3, and IgG4. Advantageously, the monoclonal antibody present in a composition used as a medicinal product as such in the context of the invention is of isotype IgG1 or IgG3, and preferably IgG1.

The Fc fragment of an antibody that may be used according to the invention may be natural, as defined here above, or indeed have been modified in various ways, provided that it comprises a functional FcR binding domain (Fc gamma receptors (FcγR) for IgGs), and preferably a functional FcRn binding domain. The modifications may include the deletion of certain parts of the Fc fragment, provided that it contains a functional FcR binding domain (Fc gamma receptors (FcγR) for IgGs), and preferably a functional FcRn binding domain. The modifications may also include various amino acid substitutions that are capable of affecting the biological properties of the antibody, provided that it contains a functional FcR binding domain, and preferably a functional FcRn binding domain. In particular, when the antibody is an IgG, it may comprise mutations designed to increase the binding to the FcγRIIIa (CD16A) receptor, as described in the documents WO00/42072, Shields et al—2001, Lazar et al—2006, WO2004/029207, WO2004/063351, and WO2004/074455. Mutations that make it possible to increase the binding to the receptor FcRn and therefore the in vivo half-life may also be present, for example as described in Shields et al—2001, Dall'Acqua et al—2002, Hinton et al—2004, Dall'Acqua et al—2006(a), WO00/42072, WO02/060919, WO2010/045193, or WO2010/106180. Other mutations, such as those that provide the ability to decrease or increase binding to the complement proteins and therefore the CDC response may or may not be present (see the documents WO99/51642, WO2004/074455, Idusogie et al—2001, Dall'Acqua et al—2006(b), and Moore et al—2010).

In one particular aspect of the invention, the preferred mutants that may be used are those comprising mutations that provide the ability to increase binding to the FcRn and thus the in vivo half-life, and are therefore mutants comprising the following combinations of mutations in their Fc fragment, as described in the document WO2010/106180:
N315D/A330V/N361D/A378V/N434Y,
P230S/N315D/M428L/N434Y,
E294del/T307P/N434Y,
T307A/N315D/A330V/E382V/N389T/N434Y,
V259I/N315D/N434Y, or
T256N/A378V/S383N/N434Y,
   wherein the numbering system for amino acids in the Fc region is the one in the EU index described in Kabat et al., Sequences of Proteins of Immunological Interest, 5th Ed. Public Health Service, National Institutes of Health, Bethesda, Md. (1991), which is incorporated herein by reference.

The term “EU index” or “Kabat EU index” is understood to refer to the numbering of amino acid residues in the human IgG1 antibody.

In another aspect of the invention, the Fc fragments of the antibodies that may be used according to the invention carry at least one mutation selected from among the following:
G316D, K326E, N315D, N361H, P396L, T350A, V284L, V323I, P352S, A378V, Y436H, V266M, N421T, G385R, K326T, H435R, K447N, N434K, K334N, V397M, E283G, A378T, F423L, A431V, F423S, N325S, P343S, K290E, S375R, F405V, K322E, K340E, N389S, F243I, T307P, N389T, S442F, K248E, Y349H, N286I, T359A, S383R, K334R, T394P, V259A, T393A, P352L, Q418P, V302A, L398P, F423P, S442P, V363I, S383N, S254F, K320E, G402D, I253F, V284A, A431T, N315H, Y319H, C226Y, F405L, T393I, N434S, R255W, A287T, N286Y, A231V, K274R, V308G, K414R, M428T, E345G, F243L, P247T, Q362R, S440N, Y278H, D312G, V262A, V305A, K246R, V308I, E380G, N276S, K439Q, S267G, F423Y, A231T, K320R, L410R, K320M, V412M, T307N, T366A, P230S, Y349S, A339T, K246E, K274E, A231P, I336T, S298N, L234P, S267N, V263A, E333G, V308A, K439R, K392R, S440G, V397I, I336V, Y373D, K288E, L309P, P227S, V379A, K288R, K320T, V282A, I377T, N421S et C261R,
   the numbering system being that of the EU index or equivalent as in Kabat.

In another aspect of the invention, the Fc fragments of the antibodies that may be used carry at least one combination of two mutations, the said combination being selected from the following:
(i) a mutation selected from among 307N, 326E, 326T, 334N, 334R, 352L, 378V, 378T, 394P, 396L, 397M and 421T and;
(ii) at least one mutation selected from among 226Y, 227S, 230S, 231V, 234P, 243I, 243L, 246R, 246E, 247T, 248E, 253F, 254F, 255W, 259A, 261R, 262A, 263A, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434S, 435R, 436H, 439R, 440G, 440N, 442F, 442P, and 447N,
   the numbering system being that of the EU index or equivalent as in Kabat and with the proviso that the mutation (i) does not occur on the same amino acid as the mutation (ii).

Preferably, the mutated Fc fragments of the antibodies that may be used according to the invention have an increased affinity for the complement C1q, and comprise at least one combination of two mutations, the said combination comprising:
i) one mutation selected from among 378V, 378T, 396L, 421T, 334R and 326E and
ii) at least one mutation selected from among 361H, 290E, 316D, 248E, 410R, 421T, 334R, 394P, 307P, 447N, 378V, 284L, 421T, 396L, 286I, 315D and 397m,
   the numbering system being that of the EU index or equivalent as in Kabat and with the proviso that the mutation (i) does not occur on the same amino acid as the mutation (ii).

Preferably, the mutated Fc fragments of the antibodies that may be used according to the invention have an increased affinity for the receptor FcγRIIIa (CD16A), and comprise at least one combination of two mutations, the said combination comprising:
i) one mutation selected from among 378V, 326E, 397M, 334N and 334N; and
ii) at least one mutation selected from among 316D, 397M, 334N, 248E, 231V, 246R, 336T, 421T, 361H, 366A, 439R, 290E, 394P, 307P, 378V, 378T, 2861, 286Y and 298N,
   the numbering system being that of the EU index or equivalent as in Kabat and with the proviso that the mutation (i) does not occur on the same amino acid as the mutation (ii).

Preferably, the mutated Fc fragments of the antibodies that may be used according to the invention have an increased affinity for the receptor FcγRIIa (CD32A), and comprise at least one combination of two mutations, the said combination comprising:
i) one mutation selected from among 378V, 326E, 397M, 307N, 394P, 326T, 396L and 334N; and
ii) at least one mutation selected from among: 316D, 334R, 334N, 323I, 231V, 246R, 336T, 378T, 286Y, 286I, 352S, 383R, 359A, 421T, 361H, 315D, 366A, 290E, 307P and 439R,
   the numbering system being that of the EU index or equivalent as in Kabat and with the proviso that the mutation (i) does not occur on the same amino acid as the mutation (ii).

Preferably, the mutated Fc fragments of the antibodies that may be used according to the invention comprise at least one combination of three mutations, the said combination comprising:
(i) one mutation selected from among 326E, 326T, 352L, 378V, 378T, 396L, 397M, 421T, 334N, 334R, 307N and 394P; and
(ii) at least two mutations selected from among 226Y, 227S, 230S, 231V, 234P, 243I, 243L, 246R, 246E, 247T, 248E, 253F, 254F, 255W, 259A, 261R, 262A, 263A, 266M, 267N, 267G, 274E, 274R, 276S, 278H, 282A, 283G, 284L, 286I, 286Y, 287T, 288E, 288R, 290E, 298N, 302A, 305A, 307P, 308A, 308I, 308G, 309P, 312G, 315D, 316D, 319H, 320T, 320R, 320M, 322E, 323I, 325S, 333G, 334N, 334R, 336T, 339T, 340E, 343S, 345G, 349S, 349H, 350A 352S, 359A, 361H, 362R, 363I, 366A, 373D, 375R, 377T, 378V, 378T, 379A, 380G, 383R, 385R, 389S, 389T, 392R, 393A, 393I, 394P, 396L, 397I, 397M, 398P, 405V, 405L, 410R, 412M, 414R, 421T, 421S, 423L, 423Y, 423S, 423P, 428T, 431V, 431T, 434K, 434S, 435R, 436H, 439R, 440G, 440N, 442F, 442P and 447N,
   the numbering system being that of the EU index or equivalent as in Kabat and with the proviso that the mutation (i) does not occur on the same amino acid as the mutation (ii).

An antibody, functional fragment or derivative thereof for use thereof according to the invention, which is chimeric with human constant regions, or indeed humanised, will advantageously comprise a human heavy chain constant region having as the amino acid sequence, SEQ ID NO: 53. Additionally or alternatively, an antibody, functional fragment or derivative thereof for use thereof according to the invention, which is chimeric with human constant regions, or indeed humanised, will advantageously comprise a human light chain constant region having as the amino acid sequence, SEQ ID NO: 54. The preferred human heavy or light chain constant region sequences, SEQ ID NO: 53 and SEQ ID NO: 54, of IgG1 isotype, are presented in Table 5 below.

TABLE 5
Preferred human heavy or light chain constant region
sequences SEQ ID NO: 53 and SEQ ID NO: 54.

| | |
|---|---|
| Preferred | ALTSGVHTFPAVLQSSGLYSLSSVVTVPSSSLGTQTYICNVNH |
| human heavy | KPSNTKVDKKVEPKSCDKTHTCPPCPAPELLGGPSVFLFPPK |
| chain constant | PKDTLMISRTPEVTCVVVDVSHEDPEVKFNWYVDGVEVHNAK |
| region (IgG1) | TKPREEQYNSTYRVVSVLTVLHQDWLNGKEYKCKVSNKALPA |
| | PIEKTISKAKGQPREPQVYTLPPSRDELTKNQVSLTCLVKGFY |
| | PSDIAVEWESNGQPENNYKTTPPVLDSDGSFFLYSKLTVDKS |
| | RWQQGNVFSCSVMHEALHNHYTQKSLSLSPG (SEQ ID NO: 53) |
| Preferred | RTVAAPSVFIFPPSDEQLKSGTASVVCLLNNFYPREAKVQWKV |
| human light | DNALQSGNSQESVTEQDSKDSTYSLSSTLTLSKADYEKHKVY |
| chain constant | ACEVTHQGLSSPVTKSFNRGEC (SEQ ID NO: 54) |
| region (IgG1) | |

Thus, the heavy and light chains of antibodies, functional fragments or derivatives thereof for use thereof according to the invention advantageously comprise the sequences described in Table 6 below.

TABLE 6
Heavy and light chain amino acid sequences of the antibodies
according to the invention.

| Antibody | Heavy Chain | Light Chain |
|---|---|---|
| 122A2 | Fusion SEQ ID NO: 43-SEQ | Fusion SEQ ID NO: 48-SEQ |
| | ID NO: 53 | ID NO: 54 |
| | (SEQ ID NO: 55) | (SEQ ID NO: 60) |
| 102E9 | Fusion SEQ ID NO: 44-SEQ | Fusion SEQ ID NO: 49-SEQ |
| | ID NO: 53 | ID NO: 54 |
| | (SEQ ID NO: 56) | (SEQ ID NO: 61) |
| 104C12 | Fusion SEQ ID NO: 45-SEQ | Fusion SEQ ID NO: 50-SEQ |
| | ID NO: 53 | ID NO: 54 |
| | (SEQ ID NO: 57) | (SEQ ID NO: 62) |
| 114D11 | Fusion SEQ ID NO: 46-SEQ | Fusion SEQ ID NO: 51-SEQ |
| | ID NO: 53 | ID NO: 54 |
| | (SEQ ID NO: 58) | (SEQ ID NO: 63) |
| 104E10 | Fusion SEQ ID NO: 47-SEQ | Fusion SEQ ID NO: 52-SEQ |
| | ID NO: 53 | ID NO: 54 |
| | (SEQ ID NO: 59) | (SEQ ID NO: 64) |

The heavy chain and/or the light chain of the antibody, functional fragment or derivative thereof for use thereof according to the invention advantageously further comprises at least one heterologous signal peptide of the sequence SEQ ID NO: 65 (MRWSWIFLLLLSITSANA, signal peptide MB7). In fact, this peptide has been shown to provide the ability to improve the expression and secretion of recombinant proteins in higher eukaryotic cell lines (see WO2011/114063). Thus, the heavy chains of antibodies, functional fragments or derivatives thereof for use thereof according to the invention advantageously comprise an amino acid sequence selected from among the sequences SEQ ID NO: 66 to70, consisting of the N- to C-terminal fusion between the amino acid sequence of signal peptide MB7 (SEQ ID NO: 65) and one of the amino acid sequences of the VH region of the antibodies according to the invention (SEQ ID NO: 43 to 47). Additionally or alternatively, the light chains of antibodies, functional fragments or derivatives thereof for use thereof according to the invention advantageously comprise an amino acid sequence selected from the sequences SEQ ID NO: 71 to 75, consisting of the N- to C-terminal fusion between the amino acid sequence of signal peptide MB7 (SEQ ID NO: 65) and one of the amino acid sequences of the VL region of the antibodies according to the invention (SEQ ID NO: 48 to 52). By adding the preferred heavy and light chain constant regions, it is possible to obtain the preferred complete amino acid sequences of the antibodies for use thereof according to the invention, as described in Table 7 below.

TABLE 7
Heavy and light chain amino acid sequences of the antibodies
according to the invention, with signal peptide MB7.

| Antibody | Heavy Chain | Light Chain |
|---|---|---|
| 122A2 | Fusion SEQ ID NO: 65-SEQ | Fusion SEQ ID NO: 65-SEQ |
| | ID NO: 43-SEQ ID NO: 53 | ID NO: 48-SEQ ID NO: 54 |
| | (SEQ ID NO: 76) | (SEQ ID NO: 81) |
| 102E9 | Fusion SEQ ID NO: 65-SEQ | Fusion SEQ ID NO: 65-SEQ |
| | ID NO: 44-SEQ ID NO: 53 | ID NO: 49-SEQ ID NO: 54 |
| | (SEQ ID NO: 77) | (SEQ ID NO: 82) |
| 104C12 | Fusion SEQ ID NO: 65-SEQ | Fusion SEQ ID NO: 65-SEQ |
| | ID NO: 45-SEQ ID NO: 53 | ID NO: 50-SEQ ID NO: 54 |
| | (SEQ ID NO: 78) | (SEQ ID NO: 83) |
| 114D11 | Fusion SEQ ID NO: 65-SEQ | Fusion SEQ ID NO: 65-SEQ |
| | ID NO: 46-SEQ ID NO: 53 | ID NO: 51-SEQ ID NO: 54 |
| | (SEQ ID NO: 79) | (SEQ ID NO: 84) |
| 104E10 | Fusion SEQ ID NO: 65-SEQ | Fusion SEQ ID NO: 65-SEQ |
| | ID NO: 47-SEQ ID NO: 53 | ID NO: 52-SEQ ID NO: 54 |
| | (SEQ ID NO: 80) | (SEQ ID NO: 85) |

In one particular aspect of the invention, the said antibody for the aforementioned use thereof has a low fucose content that is less than or equal to 65%.

In one particular aspect of the invention, the said antibody for the aforementioned use thereof has an oligomannose-type N-glycans content that is greater than or equal to 30%.

In one particular aspect of the invention, the said antibody for the aforementioned use thereof has a galactose content that is greater than or equal to 50%.

For the purposes of the present invention, the antibody, functional fragment or derivative thereof, advantageously has a low fucose content that is less than or equal to 65%.

The term “fucose content” refers to the percentage of fucosylated forms within N-glycans attached to the Asn297 residue of the Fc region of each heavy chain of each antibody.

The term “low fucose content” refers to a fucose content that is less than or equal to 65%. Indeed, it is now known that the fucose content of an antibody composition plays a crucial role in the capacity of this composition to induce a strong ADCC response via the FcγRIII.

Advantageously, the fucose content is less than or equal to 65%, preferably less than or equal to 60%, 55% or 50%, or even less than or equal to 45%, 40%, 35%, 30%, 25% or 20%. However, it is not necessary for the fucose content to be nil, and it may for example be greater than or equal to 5%, 10%, 15% or 20%. The fucose content may for example be between 5 and 65%, between 5 and 60%, between 5 and 55%, between 5 and 50%, between 5 and 45%, between 5 and 40%, between 5 and 35%, between 5 and 30%, between 5 and 25%, between 5 and 20%, between 10 and 65%, between 10 and 60%, between 10 and 55%, between 10 and 50%, between 10 and 45%, between 10 and 40%, between 10 and 35%, between 10 and 30%, between 10 and 25%, between 10 and 20%, between 15 and 65%, between 15 and 60%, between 15 and 55%, between 15 and 50% between 15 and 45%, between 15 and 40%, between 15 and 35%, between 15 and 30%, between 15 and 25%, between 15 and 20%, between 20 and 65%, between 20 and 60%, between 20 and 55%, between 20 and 50%, between 20 and 45%, between 20 and 40%, between 20 and 35%, between 20 and 30%, between 20 and 25%.

The antibody, functional fragment or derivative thereof according to the invention may moreover have different types of glycosylation (N-glycans of the oligomannose or biantennary complex type, with a variable proportion of bisecting N-acetylglucosamine (GlcNAc) residues, or of galactose residues in the case of N-glycans of the biantennary complex type), provided that they have a low fucose content. Thus, oligomannose type N-glycans may be obtained by culturing in the presence of various different glycosylation inhibitors, such as α1,2-mannosidase I inhibitors (such as Deoxymannojirimycin or “DMM”) or α-glucosidase inhibitors (such as castanospermine or “Cs”); or indeed by production of the antibody in CHO cell line Lec1. Production in the milk of transgenic goats also leads to the obtaining of antibodies, wherein the majority N-glycan is of the oligomannose type, with as minority forms fucosylated biantennary complex forms with one or two galactoses, without bisecting GlcNAc and without sialylation (G1F or G2F) (see WO2007/048077). N-glycans of the biantennary complex type may be obtained in most mammalian cells, but also in bacteria, yeasts or plants whose glycosylation machinery has been modified. To limit the fucose content, cell lines naturally having low activity of the enzyme 1,6-fucosyltransferase (FUT8) responsible for the addition of fucose on the GlcNAc bound to the Fc fragment; such as the YB2/0 cell line, duck embryonic cell line EB66®, rat hepatoma cell lines H4-II-E (DSM ACC3129) and H4-II-Es (DSM ACC3130) or the cell lines NM-H9D8-E6 (DSM ACC 2807), and NM H9D8-E6Q12 (DSM ACC 2856) may be used. Mutant lines for other genes whose underexpression or overexpression leads to a low fucose content may also be used, such as the CHO line Lec13, a mutant of the CHO cell line having decreased synthesis of GDP-fucose. It is also possible to select a cell line of interest and to decrease or abolish (in particular by use of interfering RNAs or by mutation or deletion of the gene expressing the protein of interest) the expression of a protein involved in the N-glycan fucosylation pathway (in particular FUT8, see Yamane-Ohnuki et al—2004 , but also GMD, a gene involved in GDP-fucose transport, see Kanda et al 2007). Another alternative consists in selecting a cell line of interest and in overexpressing a protein that interferes in some way with the fucosylation of N-glycans, such as the protein GnTIII (β(1,4)-N-acetylglucosaminetransferase III). In particular, antibodies having low fucosylated N-glycans were obtained in particular by:
Production in YB2/0 (see EP1176195A1, WO01/77181, Shinkawa et al, 2003) CHO Lec13 (see Shields et al, 2002), EB66® (Olivier et al, 2010), the rat hepatoma cell lines H4-II-E (DSM ACC3129), H4-II-Es (DSM ACC3130) (see WO2012/041768), and the human cell lines NM-H9D8 (DSM ACC2806), NM-H9D8-E6 (DSM ACC 2807), and NM H9D8-E6Q12 (DSM ACC 2856) (see WO2008/028686).
Production in a wild-type CHO cell line in the presence of small interfering RNAs directed against FUT8 (Mori et al, 2004, Suzuki et al, 2007, Cardarelli et al, 2009, Cardarelli et al, 2010, Herbst et al 2010), or GMD (gene encoding for the GDP-fucose transporter in the Golgi apparatus, see Imai-Nishiya et al—2007)
Production in a CHO cell line in which the two alleles of the FUT8 gene encoding for 1,6-fucosyltransferase have been deleted (Yamane-Ohnuki et al—2004), or in which the two alleles of the GMD gene encoding for the GDP-fucose transporter in the Golgi apparatus have been deleted (Kanda et al, 2007),
Production in a CHO cell line in which the gene encoding for the GnTIII (β(1,4)-N-acetylglucosaminyl-transferase III) enzyme was overexpressed transgenically (Umana et al, 1999). In addition to low fucosylation, the N-glycans obtained are characterised by a high bisecting GlcNAc content.
Production in transgenic plants (N. benthamiana), with a strong reduction of the β1,2-xylose and α1,3-fucose residue contents by means of the use of small interfering RNAs (Forthal et al.—2010).

Oligomannose-type N-glycans have a reduced in vivo half-life as compared to biantennary complex-type N-glycans. As a consequence, advantageously, the antibodies according to the invention have, on their Fc-fragment N-glycosylation sites, biantennary complex-type glycan structures with a low fucose content as defined above.

In particular, the monoclonal antibodies according to the invention may have a content of G0+G1+G0F+G1F glycoforms that is greater than 60% and a low fucose content as defined above. It may also have a content of G0+G1+G0F+G1F glycoforms that is greater than 65% and low fucose content as defined above. It may also have a content of G0+G1+G0F+G1F glycoforms that is greater than greater than 70% and a low fucose content as defined above. It may also have a content of G0+G1+G0F+G1F glycoforms that is greater than 75% and a low fucose content as defined above. It may also have a content of G0+G1+G0F+G1F glycoforms that is greater than 80% and a low fucose content as defined above. It may also have a content of G0+G1+G0F+G1F glycoforms that is greater than 60%, 65%, 70%, 75% or 80% and a content of glycoforms G0F+G1F that is less than 50%. The glycoforms G0, G1, G0F and G1F are as defined here below:

Such antibody compositions may in particular be obtained by production in YB2/0, in CHO Lec13, in wild-type CHO cell lines cultured in the presence of small interfering RNAs directed against FUT8 or GMD, in CHO cell lines in which the two alleles of the FUT8 gene encoding for 1,6-fucosyltransferase or the two alleles of the GMD gene encoding for the GDP-fucose transporter in the Golgi apparatus have been deleted.

However, in another embodiment, the antibody, functional fragment or derivative thereof according to the invention has a high oligomannose-type N-glycans content.

The term “oligomannose-type N-glycans” is understood to refer to N-glycans whose pentasaccharide core, comprising of two N-acetylglucosamine (GlcNAc) residues (one of them being bound to the Asn297 residue of the Fc region of the antibody) and three mannose residues, is supplemented by one to six additional mannoses bound to the terminal mannose residues of the pentasaccharide core. The oligomannose-type N-glycans are not fucosylated.

The term “oligomannose-type N-glycans content” refers to the percentage of oligomannose forms within N-glycans attached to the Asn297 residue of the Fc fragment of each heavy chain of each antibody. The term “high oligomannose-type N-glycans content” refers to an oligomannose-type N-glycans content that is greater than or equal to 30%, advantageously greater than or equal to 35%, greater than or equal to 40%, greater than or equal to 45%, greater than or equal to 50%, greater than or equal to 55%, greater than or equal to 60%, greater than or equal to 65% greater than or equal to 70%, greater than or equal to 75%, greater than or equal to 80%, greater than or equal to 85%, greater than or equal to 90%, or even greater than or equal to 95%.

In addition or alternatively to a low fucose content, the antibody, functional fragment or derivative thereof according to the invention has a high galactose content.

The term “galactose content” or “galactosylation level” of the antibody, refers to a percentage that is calculated from an analytical chromatogram of the N-glycans released by the antibody, according to the following formula:

$$galactose content = \frac{{\sum }_{i = 1}^{n} ( number of Gal ) * ( \% relative surface area )}{{\sum }_{i = 1}^{n} ( number of A ) * ( \% relative surface area )} * 100$$

wherein
“n” represents the number of N-glycans peaks analysed on a chromatogram, for example, with a normal phase high performance liquid chromatography spectrum (NP HPLC),
“number of Gal” represents the number of galactoses on the antenna of the glycan corresponding to the peak,
“number of A” represents the number of N-acetyl-glucosamine antennas of the glycan form corresponding to the peak, and
“% relative surface area” is the percentage of the area under the corresponding peak.

The term “high galactose content” refers to a galactose content that is greater than or equal to 30%, advantageously greater than or equal to 50%, advantageously greater than or equal to 55%, greater than or equal to 60%, greater than or equal to 65%, greater than or equal to 70%, greater than or equal to 75%, greater than or equal to 80%, greater than or equal to 85%, greater than or equal to 90%, greater than or equal to 95%, or even equal to 100%.

The invention also relates to an antibody fragment directed against the CD303 protein as defined above for use thereof.

The term “fragment” refers to the fragments Fab, F(ab′)2, Fd, scFv, scFv dimer, diabody, triabody or tetrabody.

The term “Fab” refers to an antibody fragment having molecular weight of about 50,000 dalton and having an antigen binding activity. It is approximately comprised of the N-terminal half of the heavy chain and the entire light chain linked by a disulfide bond. The Fab may be obtained in particular by treating the IgG with a protease, papain.

The term “F(ab′)2” refers to a fragment of about 100,000 dalton and having an antigen binding activity. It corresponds to the binding via a disulfide bond of two Fab fragments described above. It may be obtained by treating IgG with a protease, pepsin.

The term “Fd” corresponds to the part of the heavy chain which is included in the Fab fragment. The Fd fragment is thus formed by the VH and CH1 domains.

The term “scFv” (single chain Fv) indicates a VH:VL polypeptide synthesised by using genes encoding for the VL and VH domains and a sequence encoding for a peptide intended to bind these domains. An scFv according to the invention includes CDRs maintained in an appropriate conformation, for example by using suitable genetic recombination techniques.

The term “ScFv dimers” is understood to refer to two scFv molecules bound together by a peptide bond.

The scFv may also be used as basic modules for the development of multimeric structures (dimeric: “diabody”, trimeric: “triabody”, tetrameric: “tetrabody”).

The term “diabody” is understood to refer to an scFv dimer. This fragment dimer has the property of being able to maintain the double valence that the parent antibody possesses. The diabody is bivalent, and mono- or bispecific depending on whether it binds two identical or different antigens.

The term “triabody” is understood to refer to the trivalent combination of scFv. A triabody can thus bind three identical or different antigens.

The term “tetrabody” is understood to refer to the tetravalent combination of scFv. A tetrabody may bind four identical or different antigens.

In one particular aspect of the invention, the said antibody or said fragment for the above-mentioned use thereof is conjugated with a bioactive molecule selected from among the following:
radioactive isotopes, in particular selected from At211, I131, I125, Y90, Re186, Re188, Sm153, Bi212, P32,
non-radioactive metals,
toxins, in particular selected from ricin, abrin, diphtheria toxin,
nucleic acids, in particular selected from antisense RNAs,
enzymes, in particular selected from RNases, biotin , avidin or streptavidin,
cytotoxic agents, in particular selected from among:
   antifolates, and more particularly methotrexate, pemetrexed, raltitrexed;
   anti-purines, and more particularly cladribine, fludarabine, azathioprine, azathioprine, mercaptopurine, 5-fluorouracil, capecitabine, cytarabine, gemcitabine,
   topoisomerase I and II inhibitors,
   alkylating agents and related agents, and more particularly selected from among chlormethine, cyclophosphamide, ifosfamide, carmustine, fotemustine, mitomycin C, cisplatin, carboplatin, oxaliplatin,
   intercalating agents,
   anthracyclines, and more particularly selected from daunorubicin, doxorubicin, hydrochloride, epirubicin, idarubicin, bleomycin,
   taxanes,
   specific inhibitors of tyrosine kinase, imatinib, erlotinib.

The present invention also relates to a nucleic acid (also known as nucleic acid or nucleotide sequence) encoding for the heavy chain and/or light chain of an antibody, functional fragment or derivative thereof according to the invention as described above.

All of the different nucleic acid sequences, due to the degeneracy of the genetic code, coding for a particular amino acid sequence are within the scope of the invention. In particular, the sequence of a nucleic acid according to the invention may have been optimised to promote its expression in a host cell, a transgenic non-human animal or a transgenic plant of interest. Indeed, there are in general several combinations of three nucleotides encoding the same amino-acid (except for methionine and tryptophan), referred to as synonymous codons. However, some of these combinations are in general preferentially used by a cell or a given organism (this is referred to as genetic code/codon usage bias). This preference depends notably on the organism that produces or from which the cell is derived. Consequently, when a protein derived from one or more organisms is produced in a heterologous organism or in a cell of such a heterologous organism, it may be useful to modify the nucleic acid sequence encoding for the protein in a manner so as to use mainly the preferred codons of the heterologous organism. Data are available in the literature concerning the codon usage preferred by various different species and the person skilled in the art knows how to optimise the expression of a given protein in an organism or a cell of a heterologous organism.

A nucleic acid according to the invention advantageously comprises at least one of the sequences SEQ ID NO: 86 to 95 as described in Table 8 below, which encode for the amino acid sequences of the VH and VL regions of the antibodies according to the invention and have been optimised for expression in cells of the species Rattus norvegicus.

TABLE 8
Preferred nucleotide sequences, optimised for expression
in cells of the species Rattus norvegicus encoding the VH
and VL regions of the antibodies, functional fragments or
derivatives thereof according to the invention.

| Antibody | VH | VL |
|---|---|---|
| 122A2 | CAGGTCCAGCTGCAGCAGTCTG | GATATCCAGATGACACAGAC |
| | GGGCTGAGCTGGTGAGGCCTG | TACATCCTCCCTGTCTGCCT |
| | GGGTCTCAGTGAAGATTTCCTG | CTCTGGGAGACAGAGTCACC |
| | CAAGGGTTCTGGCTACACATTCA | ATCAGTTGCAGGGCAAGTCA |
| | CTGATTATTCTATGCACTGGGTG | GGACATTAGCAATTATTTAAA |
| | AAGCAGAGTCATGCAAAGAGTC | CTGGTATCAGCAGAAACCAG |
| | TAGAGTGGATTGGAGTTATTAGT | ATGGAACTGTTAAACTCCTG |
| | ACTTACTATGGTGATTCTAACTA | ATCTACTACACATCAAGATTA |
| | TAACCAGAAGTTCAAGGGCAAG | CACTCAGGAGTCCCATCAAG |
| | GCCACAATGACTGTAGACAAATC | GTTCAGTGGCAGTGGGTCTG |
| | CTCCACCACAGCCTATATGGAAC | GAACAGATTATTCTCTCACC |
| | TTGCCAGACTGACATCTGAGGAT | ATTAGCAACCTGGACCAAGA |
| | TCTGCCATCTATTACTGTGCAAG | AGATATTGCCACTTACTTTTG |
| | AAATGGTAATTTCTATGTTATGG | CCAACAGGGTAATACGCTTC |
| | ACTACTGGGGTCAAGGAACCTC | CTTGGACGTTCGGTGGAGG |
| | AGTCACCGTCTCCTCA (SEQ ID | CACCAAGCTGGAAATCAAA |
| | NO: 86) | (SEQ ID NO: 91) |
| 102E9 | CAGATCCATTTGGTGCAGTCTG | CAAATTGTTCTCACCCAGTC |
| | GACCTGACCTGAAGAAGCCTGG | TCCAGCAATCATGTCTGCAT |
| | AGAGACAGTCAAGATCTCCTGC | CTCCAGGGGAGAAGGTCAC |
| | AAGGCTTCTGGTTATACCTTCAC | CATAACCTGCAGTGCCAGCT |
| | AGACTATTCAATGCACTGGGTGA | CAAGTGTAATTTACATTCACT |
| | AGCAGGCTCCAGGAAAGGGTTT | GGTTCCAGCAGAAGCCAGG |
| | AAAGTGGATGGGCTGGATAAAC | CACTTCTCCCAAACTCTGGA |
| | ACTGAGACTGGTGAACCAACATA | TTTATAGCACATCCTACCTG |
| | TGCAGATGACTTCAAGGGACGG | GCTTCTGGAGTCCCTGCTCG |
| | TTTGCCTTCTCTTTGGAAAGTTC | CTTCAGTGGCAGTGGATCTG |
| | TGCCAGCACTGCCTTTTTGCAGA | GGACCTCTTACTCTCTCACA |
| | TCAACAACCTCAAAAATGAGGAC | ATCAGCCGAATGGAGGCTGA |
| | ACGTCTACATATTTCTGTACTAG | AGATGCTGCCACTTATTACT |
| | AAATGGTTACTACGTGGGTTACT | GCCAGCAGAGGAGAAGTTA |
| | ATGCTATGGACTACTGGGGTCA | CCCGTTCACGTTCGGAGGG |
| | AGGAACCTCAGTCACCGTCTCCTCA | GGGACCAAGCTGGAAATAAAA |
| | (SEQ ID NO: 87) | (SEQ ID NO: 92) |
| 104C12 | CAGGTCCAGCTGCAGCAGTCTG | GATCTCCAGATGACACAGAC |
| | GGGCTGAGCTGGTGGGGCCTG | TCCATCCTCCCTGTCTGCCT |
| | GGGTCTCAGTGAAGATTTCCTG | CTCTGGGAGACAGAGTCACC |
| | CAAGGGTTCTGGCTACACATTCA | ATCAGTTGCAGGGCAAGTCA |
| | CTGATTATTCTATGCACTGGGTA | GGACATTAACAATTATTTAAG |
| | AAGCAGAGTCATGCAAAGAGTC | CTGGTATCAGGAGAAACCAG |
| | TAGAGTGGATTGGAGTTATTAGT | ATGGAACTTTTAAACTCCTGA |
| | CCTTACTATGGTGATACTAACTA | TCTACTACACATCAAGATTAC |
| | CAACCAGAAGTTCAAGGGCAAG | ACTCAGGAGTCCCATCAAGG |
| | GCCACAATGACTGTAGACAAATC | TTCAGTGGCAGTGGGTCTGG |
| | CTCCAGCACAGCCTATATGGAA | AACAGATTATTCTCTCACCGT |
| | CTTGCCAGTCTGACATCTGAGG | TCGCAACCTGGAACAGGAAG |
| | ATTCTGCCATCTATTTCTGTGCA | ATATTGGCACTTACTTTTGCC |
| | AGAAATGATGATTACTACAGGTT | AACAGGGTAAAACGCTTCCG |
| | TGCTTACTGGGGCCAAGGGACT | TGGACGTTCGGTGGAGGCA |
| | CTGGTCACTGTCTCTGC (SEQ ID | CCAAGCTGGAAATCAG (SEQ ID |
| | NO: 88) | NO: 93) |
| 114D11 | CAGATCCAGTTGGTGCAGTCTG | CAAATTGTTCTCACCCAGTC |
| | GACCTGAGCTGAAGAAGCCTGG | TCCAGCAATCATGTCTGCAT |
| | AGAGACAGTCAAGATCTCCTGC | CTCCAGGGGAGAAGGTCAC |
| | AAGGCTTCTGGTTATACCTTCAC | CATAACCTGCAGTGCCAGCT |
| | AGACTCTTCAATGCACTGGGTG | CAAGTGTATTTTACATGCACT |
| | CAGCAGGCTCCAAACAAGGGTT | GGTTCCAGCAGAAGCCAGG |
| | TAAAGTGGATGGGCTGGATAAA | CACTTCTCCCAAACTCTGGA |
| | CACTGAGACTGGTGGGCCAACG | TTTATAGCACATCCAACCTG |
| | TATGCAGATGATTTCAAGGGACG | GCTTCTGGAGTCCCTGCTCG |
| | GTTTGCCTTCTCTTTGGAAACCT | CTTCAGTGGCAGTGGATCTG |
| | CTGCCAGAACTGCCTATTTGCAG | GGACCTCTTACTCTCTCACA |
| | ATCAACAACCTCAAAAATGAGGA | ATCAGCCGAATGGAGGCTGA |
| | CACGGCTACATATTTCTGTGCTA | AGATGCTGCCACTTATTACT |
| | GAAATGGATACTACGTGGGGTA | GCCAGCAAAGGAGAAGTTAC |
| | CTATGCTCTGGACTACTGGGGT | CCGTACACGTTCGGAGGGG |
| | CAAGGAACCTCAGTCACCGTCTCCTCA | GGACCAAGCTGGAAATAAAA |
| | (SEQ ID NO: 89) | (SEQ ID NO: 94) |
| 104E10 | CAGATCCAGTTGGTGCAGTCTG | CAAATTGTTCTCACCCAGTC |
| | GACCTGAGCTGAAGAAGCCTGG | TCCAGCAATCATGTCTGCAT |
| | AGAGACAGTCAAGATCTCCTGC | CTCCAGGGGAGAAGGTCAC |
| | AAGGCTTCTGGTTATACCTTCAC | CATGACCTGCAGTGCCAGTT |
| | AGACTATTCAATGCACTGGGTGA | CAAGTGTAATTTACATGCACT |
| | AGCAGGCTCCAGGAAAGGGTTT | GGTTCCAGCAGAAGCCAGG |
| | AAAGTGGATGGGCTGGATAAAC | CACTTCTCCCAAACTCTGGA |
| | ACTGAGACTGGTGAGCCAACAT | TTTATAGCACATCCAACCTG |
| | ATGCAGATGACTTCAAGGGACG | GCTTCTGGAGTCCCTGCTCG |
| | GTTTGCCTTCTCTTTGGAAACCT | CTTCAGTGGCAGTGGATCTG |
| | CTGCCACCACTGCCTATTTGCAG | GGACATCTTACTCTCTCACA |
| | ATCAACAACTTCAAAAATGAGGA | ATCAGCCGAATGGAGGCTGA |
| | CACGGCTACATATTTCTGTGCTA | AGATGCTGCCACTTATTACT |
| | GAAATGGTTACTACGTGGGATAT | GCCAGCAAAGGAGAAGTTAC |
| | TATGCTATGGACTACTGGGGTCA | CCGTACACGTTCGGAGGGG |
| | AGGAACCTCAGTCACCGTCTCCTCA | GGACCAAGCTGGAAATAAAA |
| | (SEQ ID NO: 90) | (SEQ ID NO: 95) |

The preferred nucleic acid sequences encoding the heavy or light chain constant regions have also been optimised for expression in cells of the species

Rattus norvegicus and are preferably those described in Table 9 below.

TABLE 9
Preferred nucleotide sequences encoding the preferred
human heavy or light chain constant regions.

| | |
|---|---|
| Preferred | GCCTCCACCAAGGGCCCATCCGTGTTCCCCCTGGCCCCAT |
| human heavy | CCAGCAAGTCTACCTCCGGAGGCACAGCCGCCCTGGGCTG |
| chain constant | TCTGGTGAAGGACTACTTCCCCGAGCCAGTGACCGTGTCC |
| region | TGGAACTCCGGAGCCCTGACATCCGGCGTGCACACCTTCC |
| | CCGCCGTGCTGCAGTCCAGCGGCCTGTACTCTCTGTCTTC |
| | CGTGGTGACCGTGCCATCCAGCTCCCTGGGAACCCAGACA |
| | TACATCTGCAACGTGAACCACAAGCCTAGCAACACCAAGGT |
| | GGACAAGAAGGTGGAGCCTAAGAGCTGTGACAAGACACAC |
| | ACATGCCCTCCTTGTCCAGCCCCTGAGCTGCTGGGCGGCC |
| | CCTCCGTGTTCCTGTTCCCCCCCAAGCCTAAGGATACCCTG |
| | ATGATCAGCAGAACCCCCGAGGTGACCTGCGTGGTGGTGG |
| | ACGTGTCCCACGAGGATCCCGAGGTGAAGTTCAACTGGTA |
| | CGTGGACGGCGTGGAGGTGCACAACGCTAAGACCAAGCC |
| | CAGAGAGGAGCAGTACAACAGCACATACAGAGTGGTGTCT |
| | GTGCTGACCGTGCTGCACCAGGACTGGCTGAACGGGAAG |
| | GAGTACAAGTGCAAGGTGTCCAACAAGGCCCTGCCTGCCC |
| | CTATCGAGAAGACCATCTCTAAGGCTAAGGGGCAGCCCCG |
| | GGAGCCACAGGTGTACACCCTGCCACCCAGCCGCGACGA |
| | GCTGACCAAGAACCAGGTGTCCCTGACATGCCTGGTGAAG |
| | GGATTCTACCCCAGCGACATCGCCGTGGAGTGGGAGAGCA |
| | ACGGCCAGCCCGAGAACAACTACAAGACAACCCCTCCCGT |
| | GCTGGACAGCGATGGATCCTTCTTCCTGTACTCCAAGCTGA |
| | CCGTGGACAAGAGCAGGTGGCAGCAGGGAAACGTGTTCTC |
| | TTGTTCCGTGATGCACGAGGCTCTGCACAACCACTACACCC |
| | AGAAGTCCCTGAGCCTGTCTCCAGGCAAG (SEQ ID NO: 96) |
| Preferred | CGAACTGTGGCTGCACCAAGTGTCTTCATCTTTCCTCCGAG |
| human light | TGATGAGCAGCTGAAGAGCGGGACAGCTTCTGTGGTGTGT |
| chain constant | CTGCTGAATAACTTCTACCCAAGAGAAGCAAAGGTCCAGTG |
| region | GAAGGTGGACAACGCCCTGCAGTCTGGCAACTCACAGGAG |
| | TCTGTCACTGAGCAGGATTCCAAGGACAGCACTTACAGCCT |
| | GTCCAGCACCCTCACTCTGTCCAAAGCCGACTACGAAAAG |
| | CATAAGGTGTATGCTTGTGAGGTGACCCACCAGGGACTGA |
| | GCAGCCCTGTGACGAAGTCCTTCAACCGGGGCGAGTGC |
| | (SEQ ID NO: 97) |

Thus, a nucleic acid encoding the heavy chain and/or light chain of an antibody according to the invention preferably comprises at least one of the nucleic acid sequences described in Table 10 below, consisting of the 5′ to 3′ fusion:
Of one of the sequences SEQ ID NO: 86 to 90 encoding the VH region of the preferred antibodies according to the invention and the sequence SEQ ID NO: 96 encoding the preferred human heavy chain constant region; or
Of one of the sequences SEQ ID NO: 91 to 95 encoding the VL region of the preferred antibodies according to the invention and the sequence SEQ ID NO: 97 encoding the preferred human light chain constant region.

TABLE 10
Heavy and light chain amino acid sequences of the antibodies
according to the invention.

| Antibody | Heavy Chain | Light Chain |
|---|---|---|
| 122A2 | Fusion SEQ ID NO: 86-SEQ | Fusion SEQ ID NO: 91-SEQ |
| | ID NO9684 | ID NO: 97 |
| | (SEQ ID NO: 98) | (SEQ ID NO: 103) |
| 102E9 | Fusion SEQ ID NO: 87-SEQ | Fusion SEQ ID NO: 92-SEQ |
| | ID NO: 96 | ID NO: 97 |
| | (SEQ ID NO: 99) | (SEQ ID NO: 104) |
| 104C12 | Fusion SEQ ID NO: 88-SEQ | Fusion SEQ ID NO: 93-SEQ |
| | ID NO:96 | ID NO: 97 |
| | (SEQ ID NO: 100) | (SEQ ID NO: 105) |
| 114D11 | Fusion SEQ ID NO: 89-SEQ | Fusion SEQ ID NO: 94-SEQ |
| | ID NO: 96 | ID NO: 97 |
| | (SEQ ID NO: 101) | (SEQ ID NO: 106) |
| 104E10 | Fusion SEQ ID NO: 90-SEQ | Fusion SEQ ID NO: 95-SEQ |
| | ID NO: 96 | ID NO: 97 |
| | (SEQ ID NO: 102) | (SEQ ID NO: 107) |

A nucleic acid encoding for the heavy chain and/or light chain of the antibody, functional fragment or derivative thereof according to the invention advantageously comprises a nucleic acid sequence encoding heterologous signal peptide MB7 (MRWSWIFLLLLSITSANA, SEQ ID NO: 65), and in particular the nucleic acid sequence SEQ ID NO: 108 (ATGAGGTGGTCCTGGATCTTCCTGCTGCTGCTGAGCATCACCAGCGCCAAC GCC). In fact, this peptide has been shown to provide the ability to improve the expression and secretion of recombinant proteins in higher eukaryotic cell lines (see WO2011/114063). Thus, a nucleic acid encoding for the heavy chain of antibodies, functional fragments or derivatives thereof according to the invention advantageously comprises a nucleic acid sequence selected from the sequences SEQ ID NO: 109 to 113, consisting of the 5′ to 3′ fusion of the nucleic acid sequence encoding signal peptide MB7 (SEQ ID NO: 108) and one of the nucleic acid sequences encoding the VH region of the antibodies according to the invention (SEQ ID NO: 86 to 90). Additionally or alternatively, a nucleic acid encoding for the light chain of antibodies, functional fragments or derivatives thereof according to the invention advantageously comprises a nucleic acid sequence selected from the sequences SEQ ID NO: 114 to 118, consisting of the 5′ to 3′ fusion of the nucleic acid sequence encoding signal peptide MB7 (SEQ ID NO: 108) to one of the amino acid sequences of the VL region of the antibodies according to the invention (SEQ ID NO: 91 to 95). By adding the preferred heavy and light chain constant regions, the preferred complete amino acid sequences of the antibodies according to the invention are obtained, as described in Table 11 below.

TABLE 11
Nucleic acid sequences encoding the heavy and light chains of the
antibodies according to the invention, with signal peptide MB7.

| Antibody | Heavy Chain | Light Chain |
|---|---|---|
| 122A2 | Fusion SEQ ID NO: 108-SEQ | Fusion SEQ ID NO: 108-SEQ |
| | ID NO: 86-SEQ ID NO: 96 | ID NO: 91-SEQ ID NO: 97 |
| | (SEQ ID NO: 119) | (SEQ ID NO: 124) |
| 102E9 | Fusion SEQ ID NO: 108-SEQ | Fusion SEQ ID NO: 108-SEQ |
| | ID NO: 87-SEQ ID NO: 96 | ID NO: 92-SEQ ID NO: 97 |
| | (SEQ ID NO: 120) | (SEQ ID NO: 125) |
| 104C12 | Fusion SEQ ID NO: 108-SEQ | Fusion SEQ ID NO: 108-SEQ |
| | ID NO: 88-SEQ ID NO: 96 | ID NO: 93-SEQ ID NO: 97 |
| | (SEQ ID NO: 121) | (SEQ ID NO: 126) |
| 114D11 | Fusion SEQ ID NO: 108-SEQ | Fusion SEQ ID NO: 108-SEQ |
| | ID NO: 89-SEQ ID NO: 96 | ID NO: 94-SEQ ID NO: 97 |
| | (SEQ ID NO: 122) | (SEQ ID NO: 127) |
| 104E10 | Fusion SEQ ID NO: 108-SEQ | Fusion SEQ ID NO: 108-SEQ |
| | ID NO: 90-SEQ ID NO: 96 | ID NO: 95-SEQ ID NO: 97 |
| | (SEQ ID NO: 123) | (SEQ ID NO: 128) |

In one particular aspect of the invention, the said antibody or said fragment for the above-mentioned use thereof is used in combination with at least one anti-cancer agent.

In one particular aspect of the invention, the said anti-cancer agent is a chemical anti-cancer agent and/or an anti-cancer agent used in immunotherapy.

In one particular aspect of the invention, the said chemical anti-cancer agent is selected from among anti-metabolic agents, alkylating agents, intercalating agents, or molecules having an action on the mitotic spindle.

In one particular aspect of the invention, the said metabolic agents are selected from among the following:
antifolates, in particular methotrexate, raltitrexed and pemetrexed,
anti-purines, in particular mercaptopurine, thioguanine, pentostatin, cladribine and fludarabine,
anti-pyrimidines, in particular 5-fluorouracil, tegafur uracil, cytarabine and capecitabine, and
anti-metabolics, in particular hydroxycarbamide, hydroxyurea and gemcitabine.

In one particular aspect of the invention, the said alkylating agents are selected from among the following:
nitrogen mustards, in particular chlorambucil, melphelan, chlormethine, metachloroethamine, estramustine, ifosfamide and cyclophosphamide,
nitrosoureas, in particular fotemustine, lomustine, carmustine, streptozocin,
organoplatines, in particular carboplatin, cisplatin and oxaliplatin,
ethylene imines, in particular thiotepa and altretamine,
triazenes, in particular procarbazine, temozolomide and dacarbazine,
alkylating agents, in particular busulfan, mitomycin C and pipobroman.

In one particular aspect of the invention, the said intercalating agents are selected from among the following:
camptothecin derivatives, in particular irinotecan and topotecan
antrhracyclines, in particular epirubicin, daunorubicin, doxorubicin, pirarubicin and idarubicin,
intercalating agents, in particular mitoxantrone, amsacrine, elliptinium, actinomycin D, dactinomycin, etoposide, and bleomycin.

In one particular aspect of the invention, the molecules having an action on the mitotic spindle are selected from among the following:
vinca alkaloids or spindle poisons, in particular vinorelbine, vindesine, vincristine and vinblastine,
taxoids or spindle microtubule stabilising agents, in particular paclitaxel and docetaxel,
tyrosine kinase inhibitors, in particular dasatinib, erlotinib, imatinib, sorafenib and sunitinib.

The abovementioned molecules are provided purely by way of indicative examples and are not intended to limit the scope of the invention. The abovementioned compounds correspond to the respective International Nonproprietary Names (INN) and the person skilled in the art could easily find the corresponding commercial brand names offered by the various relevant suppliers.

In one particular aspect of the invention, the said anti-cancer agent used in immunotherapy is an antibody specific to the targeted tumour, ie a targeted tumour specific antibody, anti-CD123 antibody, or a TLR agonist.

In another particular aspect, the antibody directed against the CD303 protein according to the invention is used in combination with an anti-CD123 antibody and/or a TLR agonist.

In another particular aspect, the antibody directed against the CD303 protein according to the invention is used in combination with an anti-CD123 antibody and a TLR agonist.

The term “targeted tumour specific antibody” is understood to refer to an antibody directed against a marker that is specific to the tumour to be prevented or treated.

An “anti-CD123 antibody” is an antibody directed against CD123 (or IL-3Rα). Such an antibody is commercially available, for example from Miltenyi Biotec, under the item reference AC145.

The term “TLR agonist” refers to agonists of Toll-Like Receptors, in particular agonists of TLR7 and TLR9. A TLR7 agonist is for example Imiquimob, and a TLR9 agonist is for example CpG oligodeoxynucleotides (CpGODN).

In one particular aspect of the invention, the said use of the said antibody or said fragment and the said use of the said anti-cancer agent are simultaneous, separate or spread out over time.

In one particular aspect of the invention, the said use of the said antibody or said fragment is simultaneous with the said use of the said anti-cancer agent.

In one particular aspect of the invention, the said use of the said antibody or said fragment is sequential to the said use of the said anti-cancer agent.

In one particular aspect of the invention, the said use of the said antibody or said fragment is prior to the said use of the said anti-cancer agent.

In one particular aspect of the invention, the use of the said antibody directed against the CD303 protein according to the invention is simultaneous with the use of the said anti-CD123 antibody and the said TLR agonist.

In one particular aspect of the invention, the use of the said antibody directed against the CD303 protein according to the invention is sequential to the uses of the said anti-CD123 antibody and the said TLR agonist. In such a case, the use of the said anti-CD123 antibody may be sequential, simultaneous or prior to use of the said TLR agonist.

In one particular aspect of the invention, the use of the said antibody directed against the CD303 protein according to the invention is prior to the use of the said anti-CD123 antibody and the said TLR agonist. In such a case, the use of the said anti-CD123 antibody may be sequential, simultaneous or prior to the use of the said TLR agonist.

In one particular aspect of the invention, the said use of the said antibody or said fragment is coupled with radiotherapy.

In one particular aspect of the invention, the said antibody or said fragment and the said anticancer agent is coupled with radiotherapy.

In one particular aspect of the invention, the said prevention or the said treatment is effected in a patient with depletion of plasmacytoid dendritic cells.

The term “patient with depletion of plasmacytoid dendritic cells” is understood to refer to a patient in whom the number of plasmacytoid dendritic cells has been reduced by a prior treatment. This term is also used to refer to a patient in whom the number of plasmacytoid dendritic cells will be reduced by a further treatment. This therefore also refers to a patient in need of or requiring a depletion of plasmacytoid dendritic cells, that is to say a patient in whom the antibodies directed against the CD303 protein will act indirectly on the tumour so as to eliminate the plasmacytoid dendritic cells of the tumour microenvironment. In such patients, the antibodies directed against the CD303 protein will thus facilitate, stimulate or potentiate the action of the anti-cancer agents, or even present a synergy with the said anti-cancer agents.

In another aspect, the present invention also relates to a host cell, a transgenic non-human animal or a transgenic plant comprising at least one nucleic acid according to the invention or a vector according to the invention.

In one particular aspect of the invention, the antibody directed against the CD303 protein is produced by transgenesis, especially in a transgenic non-human animal or a transgenic plant.

In one particular aspect of the invention, the said anti-cancer agent is produced by transgenesis, in particular in a transgenic non-human animal or a transgenic plant.

In one particular aspect of the invention, the antibody directed against the CD303 protein and the said anti-cancer agent are produced by transgenesis, in particular in a transgenic non-human animal or a transgenic plant.

The host cell may be of prokaryotic or eukaryotic origin, and may in particular be selected from bacterial cells, insect cells, plant cells, yeast or mammalian cells. The antibody, functional fragment or derivative according to the invention may then be produced by culturing the host cell under appropriate conditions. A host cell according to the invention may in particular be obtained by transformation of a cell line by the heavy and light chain expression vector(s) of an antibody, functional fragment or derivative thereof according to the invention, and separation of the various different cell clones obtained. The transformed cell line is preferably of eukaryotic origin, and may in particular be selected from insect cells, plant cells, yeast or mammalian cells. Cell lines that are appropriate for the production of antibodies include in particular the cell lines selected from: SP2/0 YB2/0 IR983F Namalwa human myeloma; PERC6; the CHO cell lines, in particular CHO-K-1, CHO-Lec10, CHO-Lec1, CHO-Lec13, CHO Pro-5, CHO dhfr-, or the CHO cell line deleted for both alleles encoding the FUT8 gene and/or the GMD gene; Wil-2; Jurkat; Vero; Molt −4; COS-7; 293-HEK; BHK; K6H6; NSO; SP2/0-Ag 14, P3X63Ag8.653, embryonic duck cell line EB66® (Valneva); rat hepatoma lines H4-II-E (DSM ACC3129), and H4-II-Es (DSM ACC3130) (see WO2012/041768), NM-H9D8 (DSM ACC2806), NM-H9D8-E6 (DSM ACC 2807), and NM H9D8-E6Q12 (DSM ACC 2856) (see WO2008/028686).

A transgenic non-human animal according to the invention may be obtained by directly injecting the gene(s) of interest (here, the rearranged genes encoding the heavy and light chains of the antibody) into a fertilised egg (Gordon et al.—1980). A transgenic non-human animal may also be obtained by introducing the gene(s) of interest (here, the rearranged genes encoding the heavy and light chains of the antibody) into an embryonic stem cell and preparing the animal by a chimera aggregation method or a chimera injection method (see Manipulating the Mouse Embryo, A Laboratory Manual, Second edition, Cold Spring Harbor Laboratory Press (1994); Gene Targeting, A Practical Approach, IRL Press at Oxford University Press (1993)). A transgenic non-human animal may also be obtained by a cloning technique in which a nucleus, into which the gene(s) of interest (here, the rearranged genes encoding the heavy and light chains of the antibody) has/have been introduced, is transplanted into an enucleated egg (Ryan et al.—1997; Cibelli et al.—1998, WO00/26357). A transgenic non-human animal producing an antibody of interest can be prepared by the methods above. The antibody may then be accumulated in the transgenic animal and harvested, notably from the animal's milk or eggs. For producing antibodies in the milk of transgenic non-human animals, preparation methods are notably described in WO90/04036, WO95/17085, WO01/26455, WO2004/050847, WO2005/033281, WO2007/048077. Methods for purifying proteins of interest from milk are also known (see WO01/26455, WO2007/106078). The transgenic non-human animals of interest notably include mice, rabbits, rats, goats, bovines (notably cows), and poultry (notably chicken).

A transgenic plant according to the invention may be selected from any plant allowing antibody production. Numerous antibodies have already been produced in transgenic plants and the technologies required for obtaining a transgenic plant expressing an antibody of interest and for recovering the antibody are well-known to a person skilled in the art (see Stoger et al.—2002, Fisher et al.—2003, Ma et al.—2003, Schillberg et al.—2005). It is also possible to influence the glycosylation obtained in the plants in order to obtain glycosylation similar to that of natural human antibodies (without xylose), but with, in addition, slight fucosylation, for example by means of small interfering RNAs (Forthal et al.—2010).

In a second aspect, the present invention relates to a composition comprising at least two antibodies directed against the protein CD303, or fragments of the said antibodies, and at least one anti-cancer agent. Such a composition consists of a mixture of antibodies directed against the CD303 protein and at least one anti-cancer agent.

In one particular aspect of the invention, the said composition comprises at least two anti-cancer agents.

The invention may also relate to a composition comprising at least one antibody directed against the protein CD303, or fragments of the said antibodies, and at least two anti-cancer agents.

In one particular aspect of the invention, the antibody in the said composition is monoclonal or polyclonal.

In one particular aspect of the invention, the antibody in the said composition is selected from a murine antibody, a chimeric antibody, a humanised antibody or a human antibody.

In one particular aspect of the invention, the antibody in the said composition is a chimeric antibody, preferably a chimeric antibody selected from murine/human chimeric antibody or a human/macaque chimeric antibody.

In one particular aspect of the invention, the antibody in the said composition is a monoclonal antibody directed against the ectodomain of the human CD303 antigen (SEQ ID NO: 130), or a functional fragment or derivative thereof, characterised in that
c) it competes for binding to the human CD303 antigen with at least one antibody selected from among the following:
   vi) An antibody whose heavy chain variable region includes the sequence SEQ ID NO: 43 and the light chain variable region includes the sequence SEQ ID NO: 48;
   vii) An antibody whose heavy chain variable region includes the sequence SEQ ID NO: 44 and the light chain variable region includes the sequence SEQ ID NO: 49;
   viii) An antibody whose heavy chain variable region includes the sequence SEQ ID NO: 45 and the light chain variable region includes the sequence SEQ ID NO: 50;
   ix) An antibody whose heavy chain variable region includes the sequence SEQ ID NO: 46 and the light chain variable region includes the sequence SEQ ID NO: 51;
   x) An antibody whose heavy chain variable region includes the sequence SEQ ID NO: 47 and the light chain variable region includes the sequence SEQ ID NO: 52; and
d) the constant regions of the light chains and heavy chains are constant regions derived from a non-murine species.

In one particular aspect of the invention, the antibody in the said composition is a chimeric, humanised or human antibody, functional fragment or derivative thereof, and is an antibody whose heavy chain variable region is represented by the sequence SEQ ID NO: 43, or a sequence having at least 80% identity with the said SEQ ID NO: 43, and whose light chain variable region is represented by the sequence SEQ ID NO: 48, or a sequence having at least 80% identity with the said SEQ ID NO: 48 (antibody 122A2).

In one particular aspect of the invention, the antibody in the said composition is a chimeric, humanised or human antibody, functional fragment or derivative thereof, and is an antibody whose heavy chain variable region is represented by the sequence SEQ ID NO: 44, or a sequence having at least 80% identity with the said SEQ ID NO: 44, and whose light chain variable region is represented by the sequence SEQ ID NO: 49, or a sequence having at least 80% identity with the said SEQ ID NO: 49 (antibody 102E9).

In one particular aspect of the invention, the antibody in the said composition is a chimeric, humanised or human antibody, functional fragment or derivative thereof, and is an antibody whose heavy chain variable region is represented by the sequence SEQ ID NO: 45, or a sequence having at least 80% identity with the said SEQ ID NO: 45, and whose light chain variable region is represented by the sequence SEQ ID NO: 50, or a sequence having at least 80% identity with the said SEQ ID NO: 50 (antibody 104C12).

In one particular aspect of the invention, the antibody in the said composition is a chimeric, humanised or human antibody, functional fragment or derivative thereof, and is an antibody whose heavy chain variable region is represented by the sequence SEQ ID NO: 46, or a sequence having at least 80% identity with the said SEQ ID NO: 46, and whose light chain variable region is represented by the sequence SEQ ID NO: 51, or a sequence having at least 80% identity with the said SEQ ID NO: 51 (antibody 114D11).

In one particular aspect of the invention, the antibody in the said composition is a chimeric, humanised or human antibody, functional fragment or derivative thereof, and is an antibody whose heavy chain variable region is represented by the sequence SEQ ID NO: 47, or a sequence having at least 80% identity with the said SEQ ID NO: 47, and whose light chain variable region is represented by the sequence SEQ ID NO: 52, or a sequence having at least 80% identity with the said SEQ ID NO: 52 (antibody 104E10).

In one particular aspect of the invention, the antibody in the said composition is a chimeric, humanised or human antibody, functional fragment or derivative thereof, and is an antibody whose heavy chain is represented by the sequence SEQ ID NO: 55, or a sequence having at least 80% identity with the said SEQ ID NO: 55, and whose light chain is represented by the sequence SEQ ID NO: 60, or a sequence having at least 80% identity with the said SEQ ID NO: 60 (antibody 122A2).

In one particular aspect of the invention, the antibody in the said composition is a chimeric, humanised or human antibody, functional fragment or derivative thereof, and is an antibody whose heavy chain is represented by the sequence SEQ ID NO: 56, or a sequence having at least 80% identity with the said SEQ ID NO: 56, and whose light chain is represented by the sequence SEQ ID NO: 61, or a sequence having at least 80% identity with the said SEQ ID NO: 61 (antibody 102E9).

In one particular aspect of the invention, the antibody in the said composition is a chimeric, humanised or human antibody, functional fragment or derivative thereof, and is an antibody whose heavy chain is represented by the sequence SEQ ID NO: 57, or a sequence having at least 80% identity with the said SEQ ID NO: 57, and whose light chain is represented by the sequence SEQ ID NO: 62, or a sequence having at least 80% identity with the said SEQ ID NO: 62 (antibody 104C12).

In one particular aspect of the invention, the antibody in the said composition is a chimeric, humanised or human antibody, functional fragment or derivative thereof, and is an antibody whose heavy chain is represented by the sequence SEQ ID NO: 58, or a sequence having at least 80% identity with the said SEQ ID NO: 58, and whose light chain is represented by the sequence SEQ ID NO: 63, or a sequence having at least 80% identity with the said SEQ ID NO: 63 (antibody 114D11).

In one particular aspect of the invention, the antibody in the said composition is a chimeric, humanised or human antibody, functional fragment or derivative thereof, and is an antibody whose heavy chain is represented by the sequence SEQ ID NO: 59, or a sequence having at least 80% identity with the said SEQ ID NO: 59, and whose light chain is represented by the sequence SEQ ID NO: 64, or a sequence having at least 80% identity with the said SEQ ID NO: 64 (antibody 104E10).

In one particular aspect of the invention, the antibody in the said composition has a low fucose content that is less than or equal to 65%.

In one particular aspect of the invention, the antibody in the said composition has an oligomannose-type N-glycans content that is greater than or equal to 30%.

In one particular aspect of the invention, the antibody in the said composition has a galactose content that is greater than or equal to 50%.

In one particular aspect of the invention, the fragment in the said composition is selected from Fab, F(ab′)2, Fd, scFv, scFv dimer, diabody, triabody or tetrabody.

In one particular aspect of the invention, the said antibody or the said fragment in the said composition is conjugated with a bioactive molecule selected from among the following:
radioactive isotopes, in particular selected from At211, I131, I125, Y90, Re186, Re188, Sm153, Bi212, P32,
non-radioactive metals,
toxins, in particular selected from ricin, abrin, diphtheria toxin,
nucleic acids, in particular selected from antisense RNAs,
enzymes, in particular selected from RNases, biotin, avidin or streptavidin,
cytotoxic agents, in particular selected from among:
   antifolates, and more particularly methotrexate, pemetrexed, raltitrexed;
   anti-purines, and more particularly cladribine, fludarabine, azathioprine, azathioprine, mercaptopurine, 5-fluorouracil, capecitabine, cytarabine, gemcitabine,
   topoisomerase I and II inhibitors,
   alkylating agents and related agents, and more particularly selected from among chlormethine, cyclophosphamide, ifosfamide, carmustine, fotemustine, mitomycin C, cisplatin, carboplatin, oxaliplatin,
   intercalating agents,
   anthracyclines, in particular daunorubicin, doxorubicin and hydrochloride, epirubicin, idarubicin, bleomycin,
   taxanes,
   specific inhibitors of tyrosine kinase, and more particularly imatinib, erlotinib.

In one particular aspect of the invention, the said anti-cancer agent in the said composition is a chemical anti-cancer agent and/or an anti-cancer agent used in immunotherapy.

In one particular aspect of the invention, the said chemical anti-cancer agent in the said composition is selected from anti-metabolic agents, alkylating agents, intercalating agents, or molecules having an action on the mitotic spindle.

In one particular aspect of the invention, the said metabolic agents in the said composition are selected from among the following:
antifolates, in particular methotrexate, raltitrexed and pemetrexed,
anti-purines, in particular mercaptopurine, thioguanine, pentostatin, cladribine and fludarabine,
anti-pyrimidines, in particular 5-fluorouracil, tegafur uracil, cytarabine and capecitabine, and
anti-metabolics, in particular hydroxycarbamide, hydroxyurea and gemcitabine.

In one particular aspect of the invention, the said alkylating agents in the said composition are selected from among the following:
nitrogen mustards, in particular chlorambucil, melphelan, chlormethine, metachloroethamine, estramustine, ifosfamide and cyclophosphamide,
nitrosoureas, in particular fotemustine, lomustine, carmustine, streptozocin,
organoplatines, in particular carboplatin, cisplatin and oxaliplatin,
ethylene imines, in particular thiotepa and altretamine,
triazenes, in particular procarbazine, temozolomide and dacarbazine,
alkylating agents, in particular busulfan, mitomycin C and pipobroman.

In one particular aspect of the invention, the said intercalating agents in the said composition are selected from among the following:
camptothecin derivatives, in particular irinotecan and topotecan
antrhracyclines, in particular epirubicin, daunorubicin, doxorubicin, pirarubicin and idarubicin,
intercalating agents, in particular mitoxantrone, amsacrine, elliptinium, actinomycin D, dactinomycin, etoposide, and bleomycin.

In one particular aspect of the invention, the molecules having an action on the mitotic spindle in the said composition are selected from among the following:
vinca alkaloids or spindle poisons, in particular vinorelbine, vindesine, vincristine and vinblastine,
taxoids or spindle microtubule stabilising agents, in particular paclitaxel and docetaxel,
tyrosine kinase inhibitors, in particular dasatinib, erlotinib, imatinib, sorafenib and sunitinib.

In one particular aspect of the invention, the said anti-cancer agent in the said composition that is used in immunotherapy is a targeted tumour specific antibody, an anti-CD123 antibody, or a TLR agonist.

In one particular aspect of the invention, the said anti-cancer agent in the said composition that is used in immunotherapy is an anti-CD123 antibody and a TLR agonist.

In a third aspect, the present invention relates to a composition comprising at least two antibodies directed against the protein CD303, or fragments of the said antibodies, and at least one anti-cancer agent, for use in the prevention or treatment of a tumour involving activation of plasmacytoid dendritic cells in the microenvironment of the said tumour.

The present invention also relates to a composition comprising at least one antibody directed against the CD303 protein, or fragment of the said antibody, and at least two anti-cancer agents, for use thereof in the prevention or treatment of a tumour involving activation of plasmacytoid dendritic cells in the microenvironment of the said tumour.

In one particular aspect of the invention, and as previously indicated above, the said plasmacytoid dendritic cells are not the cause of the tumour.

In one particular aspect of the invention, and as previously indicated above, the tumours involving activation of plasmacytoid dendritic cells are solid tumours, in particular involving an infiltration of plasmacytoid dendritic cells in their microenvironment, or hematopoietic tumours.

In one particular aspect of the invention, and as previously indicated above, the solid tumours belong to the group of tumours of the head and neck, melanoma, urogenital cancers, breast cancer.

In one particular aspect of the invention, and as previously indicated above, the hematopoietic tumours belong to the group consisting of multiple myeloma, lymphoma, leukemia, in particular T cell leukemia.

In one particular aspect of the invention, the said antibody in the said composition for the aforementioned use thereof is monoclonal or polyclonal.

In one particular aspect of the invention the said antibody in the said composition for the aforementioned use thereof is selected from among a murine antibody, a chimeric antibody, a humanised antibody or a human antibody.

In one particular aspect of the invention, the said antibody in the said composition for the aforementioned use thereof, is a chimeric antibody and preferably a chimeric antibody selected from among a murine/human chimeric antibody or a human-macaque chimeric antibody.

In one particular aspect of the invention, the antibody in the said composition for the aforementioned use thereof is a monoclonal antibody directed against the ectodomain of the human CD303 antigen (SEQ ID NO: 130), or a functional fragment or derivative thereof, characterised in that:
e) it competes for binding to the human CD303 antigen with at least one antibody selected from among the following:
   xi) An antibody whose heavy chain variable region includes the sequence SEQ ID NO: 43 and the light chain variable region includes the sequence SEQ ID NO: 48;
   xii) An antibody whose heavy chain variable region includes the sequence SEQ ID NO: 44 and the light chain variable region includes the sequence SEQ ID NO: 49;
   xiii) An antibody whose heavy chain variable region includes the sequence SEQ ID NO: 45 and the light chain variable region includes the sequence SEQ ID NO: 50;
   xiv) An antibody whose heavy chain variable region includes the sequence SEQ ID NO: 46 and the light chain variable region includes the sequence SEQ ID NO: 51;
   xv) An antibody whose heavy chain variable region includes the sequence SEQ ID NO: 47 and the light chain variable region includes the sequence SEQ ID NO: 52; and
f) the constant regions of the light chains and heavy chains are constant regions derived from a non-murine species.

In one particular aspect of the invention, the antibody in the said composition for the aforementioned use thereof is a chimeric, humanised or human antibody, functional fragment or derivative thereof, and is an antibody whose heavy chain variable region is represented by the sequence SEQ ID NO: 43, or a sequence having at least 80% identity with the said SEQ ID NO: 43, and whose light chain variable region is represented by the sequence SEQ ID NO: 48, or a sequence having at least 80% identity with the said SEQ ID NO: 48 (antibody 122A2).

In one particular aspect of the invention, the antibody in the said composition for the aforementioned use thereof is a chimeric, humanised or human antibody, functional fragment or derivative thereof, and is an antibody whose heavy chain variable region is represented by the sequence SEQ ID NO: 44, or a sequence having at least 80% identity with the said SEQ ID NO: 44, and whose light chain variable region is represented by the sequence SEQ ID NO: 49, or a sequence having at least 80% identity with the said SEQ ID NO: 49 (antibody 102E9).

In one particular aspect of the invention, the antibody in the said composition for the aforementioned use thereof is a chimeric, humanised or human antibody, functional fragment or derivative thereof, and is an antibody whose heavy chain variable region is represented by the sequence SEQ ID NO: 45, or a sequence having at least 80% identity with the said SEQ ID NO: 45, and whose light chain variable region is represented by the sequence SEQ ID NO: 50, or a sequence having at least 80% identity with the said SEQ ID NO: 50 (antibody 104C12).

In one particular aspect of the invention, the antibody in the said composition for the aforementioned use thereof is a chimeric, humanised or human antibody, functional fragment or derivative thereof, and is an antibody whose heavy chain variable region is represented by the sequence SEQ ID NO: 46, or a sequence having at least 80% identity with the said SEQ ID NO: 46, and whose light chain variable region is represented by the sequence SEQ ID NO: 51, or a sequence having at least 80% identity with the said SEQ ID NO: 51 (antibody 114D11).

In one particular aspect of the invention, the antibody in the said composition for the aforementioned use thereof is a chimeric, humanised or human antibody, functional fragment or derivative thereof, and is an antibody whose heavy chain variable region is represented by the sequence SEQ ID NO: 47, or a sequence having at least 80% identity with the said SEQ ID NO: 47, and whose light chain variable region is represented by the sequence SEQ ID NO: 52, or a sequence having at least 80% identity with the said SEQ ID NO: 52 (antibody 104E10).

In one particular aspect of the invention, the antibody in the said composition for the aforementioned use thereof is a chimeric, humanised or human antibody, functional fragment or derivative thereof, and is an antibody whose heavy chain is represented by the sequence SEQ ID NO: 55, or a sequence having at least 80% identity with the said SEQ ID NO: 55, and whose light chain is represented by the sequence SEQ ID NO: 60, or a sequence having at least 80% identity with the said SEQ ID NO: 60 (antibody 122A2).

In one particular aspect of the invention, the antibody in the said composition for the aforementioned use thereof is a chimeric, humanised or human antibody, functional fragment or derivative thereof, and is an antibody whose heavy chain is represented by the sequence SEQ ID NO: 56, or a sequence having at least 80% identity with the said SEQ ID NO: 56, and whose light chain is represented by the sequence SEQ ID NO: 61, or a sequence having at least 80% identity with the said SEQ ID NO: 61 (antibody 102E9).

In one particular aspect of the invention, the antibody in the said composition for the aforementioned use thereof is a chimeric, humanised or human antibody, functional fragment or derivative thereof, and is an antibody whose heavy chain is represented by the sequence SEQ ID NO: 57, or a sequence having at least 80% identity with the said SEQ ID NO: 57, and whose light chain is represented by the sequence SEQ ID NO: 62, or a sequence having at least 80% identity with the said SEQ ID NO: 62 (antibody 104C12).

In one particular aspect of the invention, the antibody in the said composition for the aforementioned use thereof is a chimeric, humanised or human antibody, functional fragment or derivative thereof, and is an antibody whose heavy chain is represented by the sequence SEQ ID NO: 58, or a sequence having at least 80% identity with the said SEQ ID NO: 58, and whose light chain is represented by the sequence SEQ ID NO: 63, or a sequence having at least 80% identity with the said SEQ ID NO: 63 (antibody 114D11).

In one particular aspect of the invention, the antibody in the said composition for the aforementioned use thereof is a chimeric, humanised or human antibody, functional fragment or derivative thereof, and is an antibody whose heavy chain is represented by the sequence SEQ ID NO: 59, or a sequence having at least 80% identity with the said SEQ ID NO: 59, and whose light chain is represented by the sequence SEQ ID NO: 64, or a sequence having at least 80% identity with the said SEQ ID NO: 64 (antibody 104E10).

In one particular aspect of the invention, the said antibody in the said composition for the aforementioned use thereof, has a low fucose content that is less than or equal to 65%.

In one particular aspect of the invention, the said antibody in the said composition for the aforementioned use thereof, has an oligomannose-type N-glycans content that is greater than or equal to 30%.

In one particular aspect of the invention, the said antibody in the said composition for the aforementioned use thereof, has a galactose content that is greater than or equal to 50%.

In one particular aspect of the invention, the said antibody in the said composition for the aforementioned use thereof, is an antibody fragment.

In one particular aspect of the invention, the said fragment in the said composition for the aforementioned use thereof, is selected from Fab, F(ab′)2, Fd, scFv, scFv dimer, diabody, triabody or tetrabody.

In one particular aspect of the invention, the said antibody or the said fragment in the said composition for the aforementioned use thereof, is conjugated with a bioactive molecule selected from among the following:
radioactive isotopes, in particular selected from At211, I131, I125, Y90, Re186, Re188, Sm153, Bi212, P32,
non-radioactive metals,
toxins, in particular selected from ricin, abrin, diphtheria toxin,
nucleic acids, in particular selected from antisense RNAs,
enzymes, in particular selected from RNases, biotin, avidin or streptavidin,
cytotoxic agents, in particular selected from among:
   antifolates, and more particularly methotrexate, pemetrexed, raltitrexed;
   anti-purines, and more particularly selected from cladribine, fludarabine, azathioprine, azathioprine, mercaptopurine, 5-fluorouracil capecitabine, cytarabine, gemcitabine,
   topoisomerase 1 and 11 inhibitors,
   alkylating agents and related agents, and more particularly selected from among chlormethine, cyclophosphamide, ifosfamide, carmustine, fotemustine, mitomycin C, cisplatin, carboplatin, oxaliplatin,
   intercalating agents,
   anthracyclines, in particular daunorubicin, doxorubicin and hydrochloride, epirubicin, idarubicin, bleomycin,
   taxanes,
   specific inhibitors of tyrosine kinase, and more particularly imatinib, erlotinib.

In one particular aspect of the invention, the said anti-cancer agent in the said composition for the aforementioned use thereof, is a chemical anti-cancer agent and/or an anti-cancer agent used in immunotherapy.

In one particular aspect of the invention, the said chemical anti-cancer agent in the said composition for the aforementioned use thereof is selected from anti-metabolic agents, alkylating agents, intercalating agents, or molecules having an action on the mitotic spindle.

In one particular aspect of the invention, the said metabolic agents in the said composition for the aforementioned use thereof, are selected from among the following:
antifolates, in particular methotrexate, raltitrexed and pemetrexed,
anti-purines, in particular mercaptopurine, thioguanine, pentostatin, cladribine and fludarabine,
anti-pyrimidines, in particular 5-fluorouracil, tegafur uracil, cytarabine and capecitabine, and
anti-metabolics, in particular hydroxycarbamide, hydroxyurea and gemcitabine.

In one particular aspect of the invention, the said alkylating agents in the said composition for the aforementioned use thereof, are selected from among the following:
nitrogen mustards, in particular chlorambucil, melphelan, chlormethine, metachloroethamine, estramustine, ifosfamide and cyclophosphamide,
nitrosoureas, in particular fotemustine, lomustine, carmustine, streptozocin,
organoplatines, in particular carboplatin, cisplatin and oxaliplatin,
ethylene imines, in particular thiotepa and altretamine,
triazenes, in particular procarbazine, temozolomide and dacarbazine,
alkylating agents, in particular busulfan, mitomycin C and pipobroman.

In one particular aspect of the invention, the said intercalating agents in the said composition for the aforementioned use thereof, are selected from among the following:
camptothecin derivatives, in particular irinotecan and topotecan
antrhracyclines, in particular epirubicin, daunorubicin, doxorubicin, pirarubicin and idarubicin,
intercalating agents, in particular mitoxantrone, amsacrine, elliptinium, actinomycin D, dactinomycin, etoposide, and bleomycin.

In one particular aspect of the invention, the said molecules having an action on the mitotic spindle in the said composition for the aforementioned use thereof, are selected from among the following:
vinca alkaloids or spindle poisons, in particular vinorelbine, vindesine, vincristine and vinblastine,
taxoids or spindle microtubule stabilising agents, in particular paclitaxel and docetaxel,
tyrosine kinase inhibitors, in particular dasatinib, erlotinib, imatinib, sorafenib and sunitinib.

In one particular aspect of the invention, the said anti-cancer agent used in immunotherapy, in the said composition for the aforementioned use thereof, is a targeted tumour specific antibody, anti-CD123 antibody, or a TLR agonist.

In one particular aspect of the invention, the said anti-cancer agent used in immunotherapy, in the said composition for the aforementioned use thereof, is an anti-CD123 antibody and a TLR agonist.

In one particular aspect of the invention, the use of the said antibody or the said fragment in the said composition and the said use of the said anti-cancer agent in the said composition is simultaneous, separated or spread out over time.

In one particular aspect of the invention, the use of the said antibody or the said fragment in the said composition and the use of the said anti-cancer agent in the said composition are simultaneous.

In one particular aspect of the invention, the use of the said antibody or the said fragment in the said composition and the use of the said anti-cancer agent in the said composition are sequential.

In one particular aspect of the invention, the use of the said antibody or the said fragment in the said composition is prior to the use of the said anti-cancer agent in the said composition.

In one particular aspect of the invention, the use of the said antibody directed against the CD303 protein in the said composition is simultaneous with the use of the said anti-CD123 antibody and the said TLR agonist.

In one particular aspect of the invention, the use of the said antibody directed against the CD303 protein in the said composition is sequential to the uses of the said anti-CD123 antibody and the said TLR agonist. In such a case, the use of the said anti-CD123 antibody may be sequential, simultaneous or prior to use of the said TLR agonist.

In one particular aspect of the invention, the use of the said antibody directed against the CD303 protein in the said composition is prior to the use of the said anti-CD123 antibody and the said TLR agonist. In such a case, the use of the said anti-CD123 antibody may be sequential, simultaneous or prior to use of the said TLR agonist.

In one particular aspect of the invention, the use of the said aforementioned composition is coupled with radiotherapy.

In one particular aspect of the invention, the use of the said aforementioned composition for the prevention or treatment of a tumour involving activation of plasmacytoid dendritic cells in the microenvironment of the said tumour is effected in a patient with depletion of plasmacytoid dendritic cells or in a patient in need of or requiring a depletion of plasmacytoid dendritic cells.

In a fourth aspect, the invention relates to an anti-cancer agent for use thereof in the context of the prevention or treatment of a tumour in patients with a depletion of plasmacytoid dendritic cells or in a patient in need of or requiring a depletion of plasmacytoid dendritic cells.

In one particular aspect of the invention, the said tumour in patients with a depletion of plasmacytoid dendritic cells is a tumour involving activation of plasmacytoid dendritic cells in the microenvironment thereof, wherein the said plasmacytoid dendritic cells are not the cause of the tumour.

In one particular aspect of the invention, and as previously indicated above, the tumours involving activation of plasmacytoid dendritic cells are solid tumours, in particular involving an infiltration of plasmacytoid dendritic cells in their microenvironment, or hematopoietic tumours.

In one particular aspect of the invention, and as previously indicated above, the solid tumours belong to the group of tumours of the head and neck, melanoma, urogenital cancers, breast cancer.

In one particular aspect of the invention, and as previously indicated above, the hematopoietic tumours belong to the group consisting of multiple myeloma, lymphoma, leukemia, in particular T cell leukemia.

In one particular aspect of the invention, the said anti-cancer agent for the aforementioned use thereof, is a chemical anti-cancer agent and/or an anti-cancer agent used in immunotherapy.

In one particular aspect of the invention, the said chemical anti-cancer agent, for the aforementioned use thereof is selected from anti-metabolic agents, alkylating agents, intercalating agents, or molecules having an action on the mitotic spindle.

In one particular aspect of the invention, the said anti-cancer agent which is a metabolic agent, for the aforementioned use thereof is selected from among the following:
antifolates, in particular methotrexate, raltitrexed and pemetrexed,
anti-purines, in particular mercaptopurine, thioguanine, pentostatin, cladribine and fludarabine,
anti-pyrimidines, in particular 5-fluorouracil, tegafur uracil, cytarabine and capecitabine, and
anti-metabolics, in particular hydroxycarbamide, hydroxyurea and gemcitabine.

In one particular aspect of the invention, the said anti-cancer agent which is an alkylating agent, for the aforementioned use thereof is selected from among the following:
nitrogen mustards, in particular chlorambucil, melphelan, chlormethine, metachloroethamine, estramustine, ifosfamide and cyclophosphamide,
nitrosoureas, in particular fotemustine, lomustine, carmustine, streptozocin,
organoplatines, in particular carboplatin, cisplatin and oxaliplatin,
ethylene imines, in particular thiotepa and altretamine,
triazenes, in particular procarbazine, temozolomide and dacarbazine,
alkylating agents, in particular busulfan, mitomycin C and pipobroman.

In one particular aspect of the invention, the said anti-cancer agent which is an intercalating agent, for the aforementioned use thereof is selected from among the following:
camptothecin derivatives, in particular irinotecan and topotecan
antrhracyclines, in particular epirubicin, daunorubicin, doxorubicin, pirarubicin and idarubicin,
intercalating agents, in particular mitoxantrone, amsacrine, elliptinium, actinomycin D, dactinomycin, etoposide, and bleomycin.

In one particular aspect of the invention, the said anti-cancer agent which is a molecule having an action on the mitotic spindle, for the aforementioned use thereof is selected from among the following:
vinca alkaloids or spindle poisons, in particular vinorelbine, vindesine, vincristine and vinblastine,
taxoids or spindle microtubule stabilising agents, in particular paclitaxel and docetaxel,
tyrosine kinase inhibitors, in particular dasatinib, erlotinib, imatinib, sorafenib and sunitinib.

In one particular aspect of the invention, the said anti-cancer agent that is used in immunotherapy, for the aforementioned use thereof is selected from a targeted tumour specific antibody, an anti-CD123 antibody, or a TLR agonist.

In one particular aspect of the invention, the said anti-cancer agent that is used in immunotherapy, for the aforementioned use thereof, is an anti-CD123 antibody and a TLR agonist.

In one particular aspect of the invention, the said anti-cancer agent for the aforementioned use thereof, is used in combination with a monoclonal or polyclonal antibody, directed against the CD303 protein, or fragment thereof, in particular an antibody having a low fucose content, that is less than or equal to 65%, and/or an oligomannose-type N-glycans content that is greater than or equal to 30% and/or a galactose content that is greater than or equal to 50%.

In one particular aspect of the invention, the antibody directed against the CD303 protein used in combination with the said anti-cancer agent for the aforementioned use thereof is selected from a murine antibody, a chimeric antibody, a humanised antibody or a human antibody, preferably a chimeric antibody selected from among a murine/human chimeric antibody or a human-macaque chimeric antibody.

In one particular aspect of the invention, the antibody directed against the CD303 protein used in combination with the said anti-cancer agent for the aforementioned use thereof is a monoclonal antibody directed against the ectodomain of the human CD303 antigen (SEQ ID NO: 130), or a functional fragment or derivative thereof, characterised in that:
g) it competes for binding to the human CD303 antigen with at least one antibody selected from among the following:
   xvi) An antibody whose heavy chain variable region includes the sequence SEQ ID NO: 43 and the light chain variable region includes the sequence SEQ ID NO: 48;
   xvii) An antibody whose heavy chain variable region includes the sequence SEQ ID NO: 44 and the light chain variable region includes the sequence SEQ ID NO: 49;
   xviii) An antibody whose heavy chain variable region includes the sequence SEQ ID NO: 45 and the light chain variable region includes the sequence SEQ ID NO: 50;
   xix) An antibody whose heavy chain variable region includes the sequence SEQ ID NO: 46 and the light chain variable region includes the sequence SEQ ID NO: 51;
   xx)An antibody whose heavy chain variable region includes the sequence SEQ ID NO: 47 and the light chain variable region includes the sequence SEQ ID NO: 52; and
h) the constant regions of the light chains and heavy chains are constant regions derived from a non-murine species.

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain variable region is represented by the sequence SEQ ID NO: 43, or a sequence having at least 80% identity with the said SEQ ID NO: 43, and whose light chain variable region is represented by the sequence SEQ ID NO: 48, or a sequence having at least 80% identity with the said SEQ ID NO: 48 (antibody 122A2).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain variable region is represented by the sequence SEQ ID NO: 44, or a sequence having at least 80% identity with the said SEQ ID NO: 44, and whose light chain variable region is represented by the sequence SEQ ID NO: 49, or a sequence having at least 80% identity with the said SEQ ID NO: 49 (antibody 102E9).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain variable region is represented by the sequence SEQ ID NO: 45, or a sequence having at least 80% identity with the said SEQ ID NO: 45, and whose light chain variable region is represented by the sequence SEQ ID NO: 50, or a sequence having at least 80% identity with the said SEQ ID NO: 50 (antibody 104C12).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain variable region is represented by the sequence SEQ ID NO: 46, or a sequence having at least 80% identity with the said SEQ ID NO: 46, and whose light chain variable region is represented by the sequence SEQ ID NO: 51, or a sequence having at least 80% identity with the said SEQ ID NO: 51 (antibody 114D11).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain variable region is represented by the sequence SEQ ID NO: 47, or a sequence having at least 80% identity with the said SEQ ID NO: 47, and whose light chain variable region is represented by the sequence SEQ ID NO: 52, or a sequence having at least 80% identity with the said SEQ ID NO: 52 (antibody 104E10).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain is represented by the sequence SEQ ID NO: 55, or a sequence having at least 80% identity with the said SEQ ID NO: 55, and whose light chain is represented by the sequence SEQ ID NO: 60, or a sequence having at least 80% identity with the said SEQ ID NO: 60 (antibody 122A2).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain is represented by the sequence SEQ ID NO: 56, or a sequence having at least 80% identity with the said SEQ ID NO: 56, and whose light chain is represented by the sequence SEQ ID NO: 61, or a sequence having at least 80% identity with the said SEQ ID NO: 61 (antibody 102E9).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain is represented by the sequence SEQ ID NO: 57, or a sequence having at least 80% identity with the said SEQ ID NO: 57, and whose light chain is represented by the sequence SEQ ID NO: 62, or a sequence having at least 80% identity with the said SEQ ID NO: 62 (antibody 104C12).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain is represented by the sequence SEQ ID NO: 58, or a sequence having at least 80% identity with the said SEQ ID NO: 58, and whose light chain is represented by the sequence SEQ ID NO: 63, or a sequence having at least 80% identity with the said SEQ ID NO: 63 (antibody 114D11).

In one particular aspect of the invention, the antibody directed against the CD303 protein used in combination with the said anti-cancer agent for the aforementioned use thereof is a chimeric, humanised or human antibody, functional fragment or derivative thereof, and is an antibody whose heavy chain is represented by the sequence SEQ ID NO: 59, or a sequence having at least 80% identity with the said SEQ ID NO: 59, and whose light chain is represented by the sequence SEQ ID NO: 64, or a sequence having at least 80% identity with the said SEQ ID NO: 64 (antibody 104E10).

In one particular aspect of the invention, the antibody directed against the CD303 protein, or fragment thereof, used in combination with the said anti-cancer agent for the abovementioned use thereof, is conjugated with a bioactive molecule selected from among the following:
radioactive isotopes, in particular selected from At211, I131, I125, Y90, Re186, Re188, Sm153, Bi212, P32,
non-radioactive metals,
toxins, in particular selected from ricin, abrin, diphtheria toxin,
nucleic acids, in particular selected from antisense RNAs,
enzymes, in particular selected from RNases, biotin, avidin or streptavidin,
cytotoxic agents, in particular selected from among:
   antifolates, and more particularly methotrexate, pemetrexed, raltitrexed;
anti-purines, and more particularly cladribine, fludarabine, azathioprine, azathioprine, mercaptopurine, 5-fluorouracil, capecitabine, cytarabine, gemcitabine,
   topoisomerase 1 and 11 inhibitors,
   alkylating agents and related agents, and more particularly chlormethine, cyclophosphamide, ifosfamide, carmustine, fotemustine, mitomycin C, cisplatin, carboplatin, oxaliplatin,
   intercalating agents,
   anthracyclines, more particularly selected from daunorubicin, doxorubicin and hydrochloride, epirubicin, idarubicin, bleomycin,
   taxanes,
   specific inhibitors of tyrosine kinase, and more particularly imatinib, erlotinib.

In one particular aspect of the invention, the aforementioned use of the said anticancer agent and the said antibody or said fragment are simultaneous, separate or spread out over time.

In one particular aspect of the invention, the aforementioned use of the said antibody or said fragment is simultaneous with the use of the said anti-CD123 antibody and the said TLR agonist.

In one particular aspect of the invention, the aforementioned use of the said antibody or said fragment is sequential to the uses of the said anti-CD123 antibody and the said TLR agonist. In such a case, the use of the said anti-CD123 antibody may be sequential, simultaneous or prior to use of the said TLR agonist.

In one particular aspect of the invention, the aforementioned use of the said antibody or said fragment is prior to the uses of the said anti-CD123 antibody and the said TLR agonist. In such a case, the use of the said anti-CD123 antibody may be sequential, simultaneous or prior to use of the said TLR agonist.

In one particular aspect of the invention, the said anti-cancer agent for the aforementioned use thereof is used in combination with radiation therapy.

The invention will be better illustrated by the following examples and figures. The examples here below are aimed at clarifying the object of the invention and illustrating advantageous embodiments, while in no way being intended to limit the scope of the invention.

### LEGENDS OF THE FIGURES

FIG. 1. Maps of the expression vectors for chimeric antibodies 122A2 (A), 102E9 (B), 104C12 (C), 114D11 (D), and 104E10 (E).

FIG. 2. Antigen binding of the antibodies according to the invention. (A) Mean fluorescence intensity (MFI) of Fc Gamma chain-CD303 Jurkat cells labelled with the antibodies according to the invention, at different antibody concentrations (represented in logarithmic units). (B) Mean fluorescence intensity (MFI) of CAL-1 cells (cell line established from BPDCN patients) labelled with the chimeric antibodies according to the invention, at different antibody concentrations (represented in logarithmic units).

FIG. 3. FcγRIIIa (CD16A)-Binding. The CD16-binding of the antibodies according to the invention was studied in a competition experiment using a phycoerythrin-coupled murine anti-CD16 antibody, 3G8. The binding of anti-CD16 3G8 to CD16 (mean fluorescence intensity values) is measured as a function of the increasing concentration of antibodies according to the invention added (μg/mL).

FIG. 4. ADCC Activities induced by the antibodies according to the invention with respect to Fc Gamma chain-CD303 Jurkat cells. The percentage of lysis by ADCC (% lysis, as defined in Example 2) of Fc Gamma chain-CD303 Jurkat target cells induced by the chimeric antibodies according to the invention is represented as a function of antibody concentration (ng/mL, logarithmic scale).

FIG. 5. Depletion of NF-3C8 cells induced by the antibody 122A2 in a whole blood test with five donors. The abscissa represents the 6 tests carried out: the test performed with a control antibody and the five tests performed with the antibody 122A2, with the whole blood of five healthy donors wherein NF-3C8 cells have been added in order to mimic the blood of a patient with BPDCN (Blastic Plasmacytoid Dendritic Cell Neoplasm). The ordinate represents the percentage of depletion of NF-3C8 cells.

### EXAMPLES

### Example 1

### Preparation and Structure of Five Chimeric Antibodies

Five chimeric monoclonal antibodies, with murine variable regions and human constant regions of IgG1-type were generated and their structures characterised.

Materials and Methods

Sequencing of the Heavy and Light Chains from Murine Hybridomas

Total RNA from each hybridoma was extracted using the NucleoSpin RNA II Kit (column purification) from Macherey-Nagel.

The mRNAs were converted to cDNAs and the heavy and light chains of the antibody were amplified using the GeneRacer kit (Invitrogen) and cloned into an M13 vector. Bacteria were then transformed by the M13 vector and the clones positive for the M13 sequences were sequenced.

Determination of the Heavy Chain VH, DH, JH Segments and the Light Chain VL and JL Segments

The variable portion, the V and J segments used by the heavy and light chains and the sequences of the heavy and light chain CDRs were determined by using IMGT's Domain Gap Align tool (see Ehrenmann et al.—2010 and Ehrenmann et al.—2011) available at the following address: http://www.imgt.org/3Dstructure-DB/cgi/DomainGapAlign.cgi. Construction of Expression Vectors for the Chimeric Antibodies

The sequences of the variable regions, VH and VL, of the five murine antibodies were optimised for preferential use of codons from species Rattus norvegicus. A sequence encoding heterologous signal peptide MB7 was in addition introduced at the 5′ end of the sequence encoding the variable region, VH or VL, of each antibody.

The sequences of the human constant portions were extracted from the expression vector CHK622-21 for esxpressing a human anti-Rhesus D antibody (T125) by digestion with ApaI/AscI for the H chain (IgG1m1.17) and DraIII/XbaI for the Kappa chain.

Finally, the variable and constant portions of a same given chain were introduced simultaneously into the generic HKgenEFss vector by ligation with KAPA T4 DNA ligase.

Results

The data concerning the heavy chain VH, DH, JH segments and the light chain VL and JL segments of the five antibodies are presented in Table 2 above. It is noted that:
The two antibodies of family 1 (122A2 and 104C12) share the use of the same VH segment (IGHV1S137*01), as well as the use of the VL (IGKV10-96*01/IGKV10-96*02) and JL (IGKJ1*01/IGKJ1*02) segments of the same family, as illustrated in Table 12 below. These two antibodies thus have a similar structure.
The three antibodies of family 2 (102E9, 114D11 and 104E10) share use of the same VH, JH, VL and JL segments, as illustrated in Table 12 below. These three antibodies thus have a similar structure.

TABLE 12
The VH JH, VL and JL segments used by the various antibodies.

| | | | | |
|---|---|---|---|---|
| Antibody | VH | JH | VL | JL |

Family 1

| | | | | |
|---|---|---|---|---|
| 122A2 | IGHV1S137*01 | IGHJ2*02 | IGKV10-96*01 | IGKJ1*01 |
| 104C12 | IGHV1S137*01 | IGHJ3*01 | IGKV10-96*02 | IGKJ1*02 |

Family 2

| | | | | |
|---|---|---|---|---|
| 102E9 | IGHV9-2-1*01 | IGHJ4*01 | IGKV4-57*01 | IGKJ1*02 |
| 114D11 | IGHV9-2-1*01 | IGHJ4*01 | IGKV4-57*01 | IGKJ1*02 |
| 104E10 | IGHV9-2-1*01 | IGHJ4*01 | IGKV4-57*01 | IGKJ1*02 |

The identical segments within the same family are in bold.

Furthermore, the data concerning the amino acid sequences of the CDRs and of the variable regions of the five antibodies are presented in Table 3 above. It is noted that:
The two antibodies of family 1 (122A2 and 104C12) have the same CDR1-H and CDR2-L sequences, and in addition have highly similar CDR2-H, CDR1-L and CDR3-L sequences (a difference of only one or two amino acids). Even the CDR3-H sequences have high homology (5/11 amino acids in common), as illustrated in Table 13 below. That confirms that these two antibodies have a very similar structure.
The three antibodies of family 2 (102E9, 114D11 and 104E10) have the same CDR2-L sequences, and in addition have highly similar CDR1-H, CDR2-H, CDR1-L and CDR3-L sequences (a difference of only one amino acid). Even the CDR3-H sequences have high homology (12/14 amino acids in common), as illustrated in Table 13 below. That confirms that these three antibodies have a structure that is truly highly similar.

TABLE 13
Sequence homology between the CDRs of antibodies of the same
family. The identical amino acids within the same family
are in bold.

| | | | | | | |
|---|---|---|---|---|---|---|
| Antibody | CDR1-H | CDR2-H | CDR3-H | CDR1-L | CDR2-L | CDR3-L |

Family 1

| | | | | | | |
|---|---|---|---|---|---|---|
| 122A2 | GYTFTDYS | ISTYYGDS | ARNGNFYVMDY | QDISNY | YTS | QQGNTLP |
| | | | | | | WT |
| 104C12 | GYTFTDYS | ISPYYGDT | ARNDDYYRFAY | QDINNY | YTS | QQGKTLPWT |

Family 2

| | | | | | | |
|---|---|---|---|---|---|---|
| 102E9 | GYTFTDYS | INTETGEP | TRNGYYVGYYAM | SSVIY | STS | QQRRSYPFT |
| | | | DY | | | |
| 114D11 | GYTFTDSS | INTETGGP | ARNGYYVGYYALDY | SSVFY | STS | QQRRSYPYT |
| 104E10 | GYTFTDYS | INTETGEP | ARNGYYVGYYAM | SSVIY | STS | QQRRSYPYT |
| | | | DY | | | |

The data concerning the amino acid sequences of the constant regions of the five antibodies are presented in Table 5 shown above.

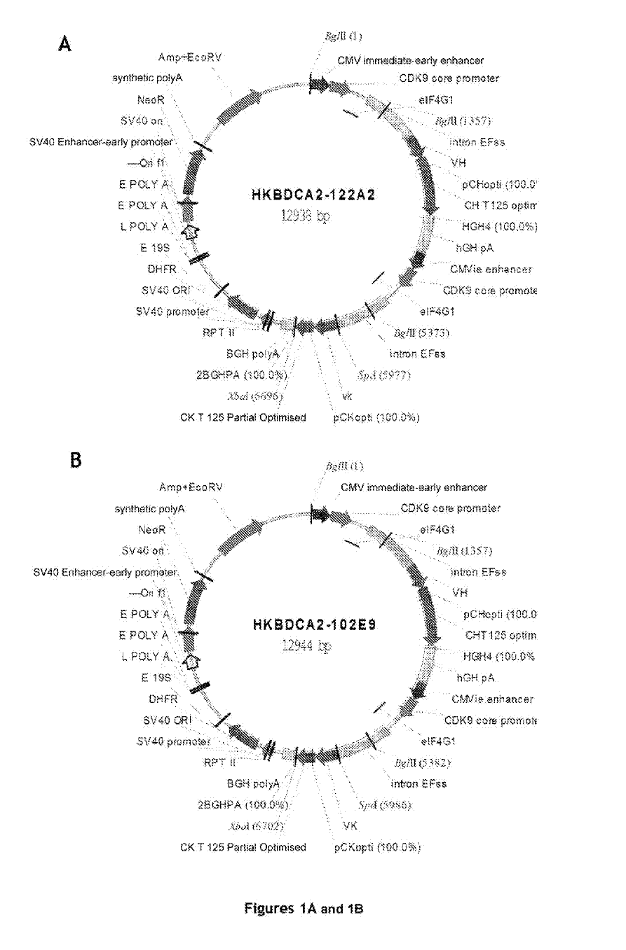

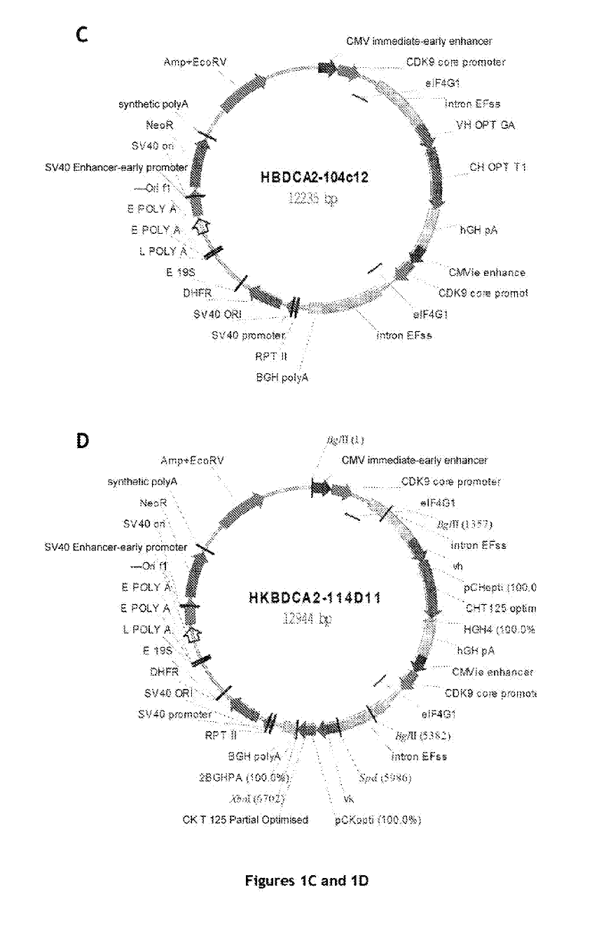

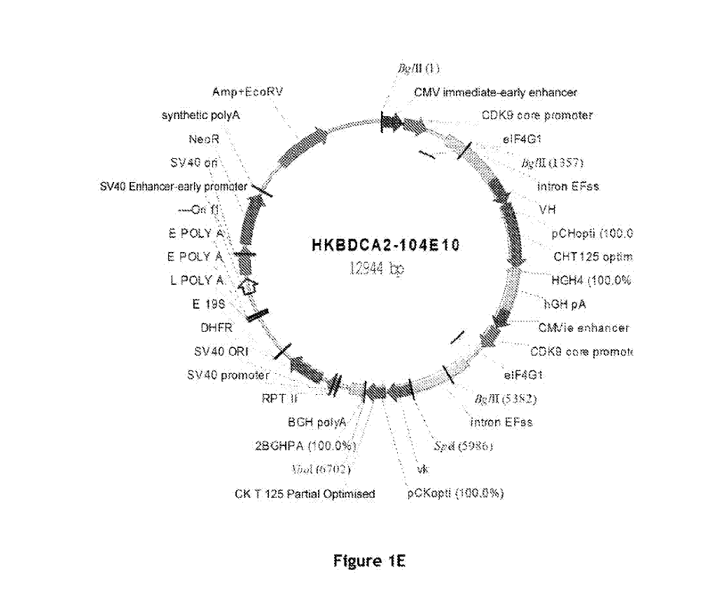

Moreover, the nucleic acid sequences of the variable regions, VH and VL, and of the constant regions of each antibody are presented in Tables 8 and 9 above. Lastly, the maps of the expression vectors for the five antibodies are presented in FIGS. 1A to 1E.

Conclusions

Five chimeric monoclonal antibodies, with murine variable regions and human constant regions of IgG1 type, and directed against the CD303 antigen, were generated and their structures characterised. It turns out that two antibodies (122A2 and 104C12) have similar structures and form an antibody subfamily (family 1), and that the other three antibodies (102E9, 114D11 and 104E10) are also very similar structurally and form another antibody subfamily (family 2).

These antibodies were then characterised in terms of their biological properties (see Example 2).

### Example 2

### Biological Properties of the Five Chimeric Antibodies

The five chimeric monoclonal antibodies, with murine variable regions and human constant regions of IgG1 type, and directed against the CD303 antigen, generated in Example 1 were tested for various biological properties.

Materials and Methods

Antigen-Binding

Binding to CD303+ cells (Fc Gamma chain-CD303 Jurkat and CAL-1)

The Fc Gamma chain-CD303 Jurkat cells (or CAL-1 cells: Maeda T et al, Int J Hematol. 2005 February; 81(2): 148-54), and the antibodies are prepared in diluent (PBS+1% FCS).

1×10⁵ cells are incubated at 4° C. for 30 minutes with 100 μL of antibody (anti-CD303 or negative control) at various concentrations (0-40 μg/mL, final concentration).

After washing with the diluent, the antibodies are visualised by addition of a phycoerythrin (PE)-coupled goat anti-mouse IgG F(ab′)₂ fragment (100 μL diluted to 1:100 in the diluent) for 45 minutes at 4° C. The cells are then washed and analysed by flow cytometry (FC500, Beckman Coulter).

FcγRIIIa (CD16a)-Binding

NK cells were isolated from peripheral blood mononuclear cells (PBMCs), then incubated with varying concentrations of the antibodies tested (0 to 100 μg/mL) simultaneously incubated with the phycoerythrin-coupled murine antibody (3G8-PE, Beckman Coulter) at 10 μL/test.

After washing, the binding of 3G8-PE to CD16 expressed by the NK cells was evaluated by flow cytometry. Mean fluorescence intensity (MFI) values are expressed as a percentage, 100% being the value obtained with the 3G8-PE antibody alone, and 0% the value obtained in the absence of 3G8-PE.

IC50 values (concentration of anti-CD303 antibody necessary to induce 50% inhibition of 3G8-binding) are calculated using the PRISM software.

ADCC

Fc Gamma chain-CD303 Jurkat cells (35,000 cells/well) are incubated in a 96-well flat-bottom plate with NK cells and increasing concentrations of anti-CD303 antibody for 4 hours at 37° C. After incubation, the supernatant is collected. Lysis of the target cells induced by the anti-CD303 antibodies is measured chromogenically by quantifying the intracellular lactate dehydrogenase (LDH) enzyme released into the supernatant by the lysed target cells (Cytotoxicity Detection Kit (LDH), Roche Diagnostics).

The percentage of lysis is calculated according to the following formula:

% lysis=[(ER−SR)/(100−SR)]−[(NC−SR)/(100−SR)]

Where ER and SR represent the experimental release (ER) and the spontaneous release (SR) of LDH, respectively, and NC represents the natural cytotoxicity of the NK cells.

The results (% lysis) are expressed as a function of antibody dilution factor. For each antibody, the “50% activity” value corresponds to the antibody dilution factor necessary to induce 50% of the plateau value obtained for this antibody. This value was calculated with the PRISM software.

Results

Antigen-Binding

Binding to Fc Gamma Chain-CD303 Jurkat Cells

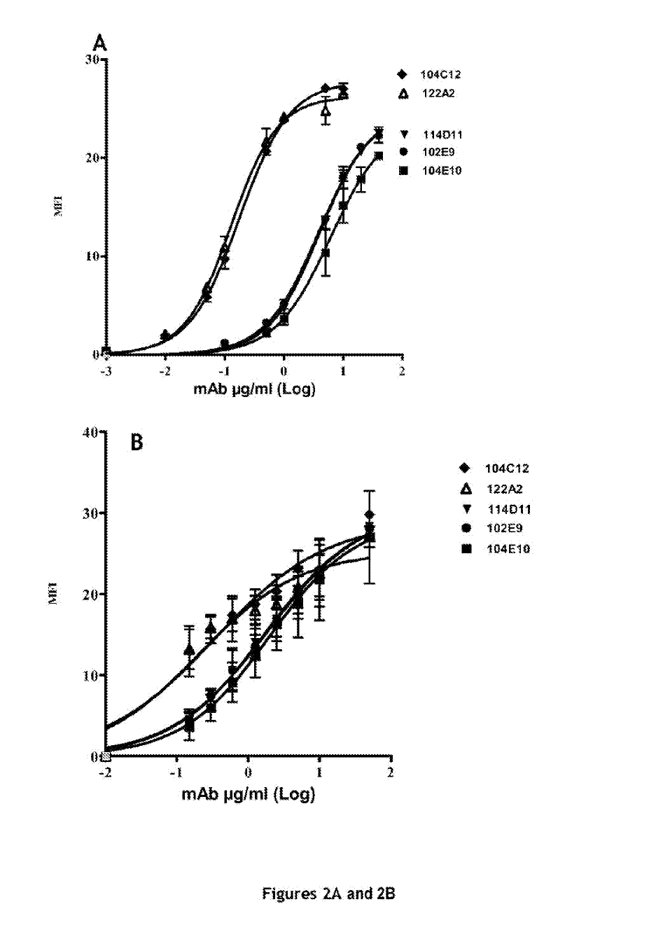

The results of the tests for binding of the antibodies according to the invention to their CD303 antigen on Fc Gamma chain-CD303 Jurkat cells are presented in FIG. 2A and in Table 14 below.

TABLE 14
Binding of the antibodies according to the invention to their CD303
antigen on Fc Gamma chain-CD303 Jurkat cells.

| | 104C12 | 122A2 | 114D11 | 102C9 | 104E10 |
|---|---|---|---|---|---|
| Bmax (MFI) | 27.74 | 26.21 | 24.69 | 24.95 | 23.73 |
| EC50 | 0.1781 | 0.1284 | 3.980 | 3.870 | 6.064 |
| (μg/mL) | | | | | |

Bmax: maximum binding expressed as mean fluorescence intensity (MFI).
EC50 (μg/mL): antibody concentration in μg/mL to obtain 50% of the maximum binding obtained for this antibody.

These relative Kd results and the Bmax values calculated after dose-response modelling make it possible to classify the antibodies into two groups: A first group that contains the antibodies 104C12 (Bmax: MFI=27.7; Kd=0.17 μg/mL) and 122A2 (Bmax: MFI=26.2 Kd=0.13 μg/mL) which are comparable and exhibit higher relative affinity than the antibodies of the second group: 114D11 (Bmax: MFI=24.7; Kd=3.9 μg/mL), 104E10 (Bmax: MFI=23.7; Kd=6 μg/mL) and 102E9 (Bmax: MFI=24.9; Kd=3.8 μg/mL).

These results show that all the chimeric antibodies generated efficiently bind the CD303 antigen expressed on the surface of Jurkat cells, for which they are specific.

Binding to CAL-1 Cells

The results of the tests for binding of the antibodies according to the invention to their CD303 antigen on CAL-1 cells are presented in FIG. 2B and in Table 15 below.

TABLE 15
Binding of the antibodies according to the invention to their CD303
antigen on the CAL-1 cells.

| | 104C12 | 122A2 | 114D11 | 102C9 | 104E10 |
|---|---|---|---|---|---|
| Bmax (MFI) | 29.02 | 25.22 | 30.14 | 30.47 | 29.2 |
| EC50 | 0.3447 | 0.2075 | 1.704 | 1.813 | 1.932 |
| (μg/mL) | | | | | |

Bmax: maximum binding expressed as mean fluorescence intensity (MFI).
EC50 (μg/mL): antibody concentration in μg/mL to obtain 50% of the maximum binding obtained for this antibody.

These relative Kd results and the Bmax values calculated after dose-response modelling make it possible to classify the antibodies into two groups: A first group that contains the antibodies 104C12 (Bmax: MFI=29.02 Kd=0.34 μg/mL) and 122A2 (Bmax: MFI=25.2 Kd=0.20 μg/mL) which are comparable and exhibit higher relative affinity than the antibodies of the second group: 114D11 (Bmax: MFI=30.1; Kd=1.7 μg/mL), 104E10 (Bmax: MFI=29.2; Kd=1.93 μg/mL) and 102E9 (Bmax: MFI=30.47; Kd=1.81 μg/mL).

These results notably correlate with the results for binding to Jurkat-CD303 cells.

FcvRIIIa (CD16a)-Binding

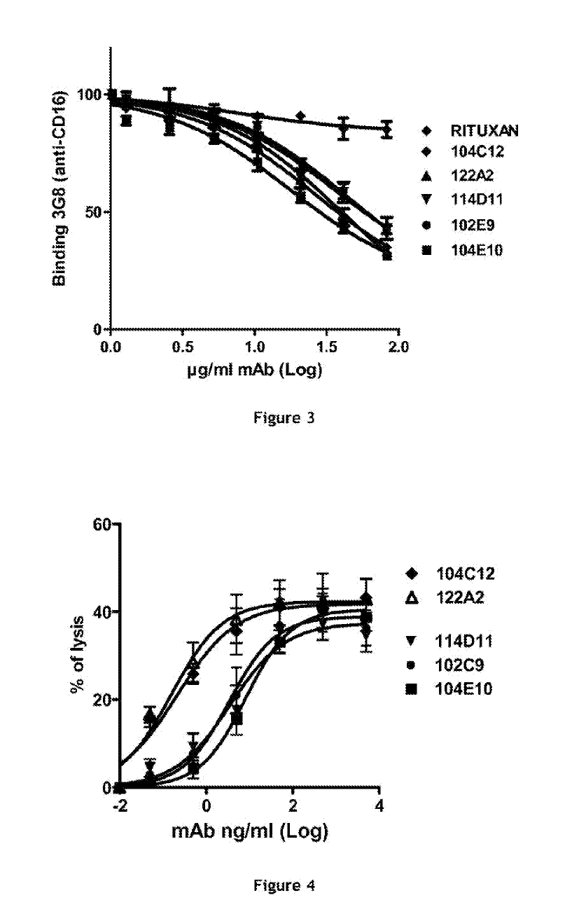

The results of the tests for binding to FcγRIIIa (CD16a) are presented in FIG. 3, and show that the chimeric antibodies according to the invention are all capable of efficiently binding CD16a, with an optimised binding affinity. They have an IC50 value below that of the antibody produced in CHO cells (Rituxan), in a range of 18.08 to 45.02 μg/mL, even below 18 μg/mL, as illustrated in Table 16 below.

TABLE 16
IC50 values (concentration of anti-CD303 antibody necessary
to induce 50% inhibition of 3G8-binding) of the control (Rituxan)
and of the chimeric antibodies of the invention

| | RITUXAN | 104C12 | 122A2 | 114D11 | 102E9 | 104E10 |
|---|---|---|---|---|---|---|
| IC50 | >80 | 26.75 | 40.44 | 45.02 | 36.99 | 18.08 |
| (μg/ml) | | | | | | |

ADCC

The results of the ADCC tests are presented in FIG. 4 and in Table 17 below.

TABLE 17
ADCC induced by the antibodies according to the invention
on Fc Gamma chain-CD303 Jurkat target cells.

| | 104C12 | 122A2 | 114D11 | 102C9 | 104E10 |
|---|---|---|---|---|---|
| Emax | 41.81 | 42.35 | 37.44 | 38.92 | 40.54 |
| (% lysis) | | | | | |
| EC50 | 0.2143 | 0.1592 | 3.612 | 3.424 | 8.280 |
| (ng/mL) | | | | | |

Emax: maximum lysis obtained with this antibody, expressed as a percentage of lysis.
EC50 (ng/mL): concentration of antibody in ng/mL to obtain 50% of the maximum lysis obtained for this antibody.

These results show that the five anti-CD303 chimeric antibodies induce lysis of Jurkat-CD303 cells (Emax about 40%). The EC50 values for 104C12 (EC50: 0.21 ng/mL), 122A2 (EC50: 0.16 ng/mL), 114D11 (EC50: 3.6 ng/mL), 102C9 (EC50: 3.4 ng/mL) and 104E10 (EC50: 8.3 ng/mL) suggest that the antibodies having high affinity are more effective with regard to ADCC than the antibodies having lower affinity.

### Example 3

### Tests for Cell Depletion in Whole Blood

The ability of the antibody 122A2 (as indicated in Example 1) to deplete NF-3C8 cells, in whole blood was tested.

Materials and Methods

NF-3C8 cells are cells derived from patients with BPDCN (Blastic Plasmacytoid Dendritic Cell Neoplasm). These cells were produced from the starting cell line CAL-1 as described in Maeda T et al. (Int J Hematol, 2005 February, 81(2): 148-54) naturally expressing the gamma chain of FccRI. After transfection of this line with CD303, the line 3C8 was selected for stably expressing 40,000 and 50,000 CD303 antigens on their surface.

Whole Blood Test

To mimic in ex vivo conditions the blood of a BPDCN patient, 500 NF-3C8 cells/μL were added to 100 μL of whole blood (in the presence of an anticoagulant: lithium heparin, derived from healthy donors, and then incubated overnight at 37° C., in sterile test tubes with 1 μg/mL of the antibody 122A2 (“Ch122A2 mAb”) or the control antibody (“control”).

Antigen Specificity and Recognition

After incubation over one night, the cells were stained directly in the test tube with CD3-FITC (anti-CD3 antibody, such as item reference A07746 from Beckman Coulter), CD123PE (an anti-CD123 antibody, such as item reference 130-090-899 from Miltenyi Biotec), CD56-PC5 (an anti-CD56 antibody, such as item reference A07789 from Beckman Coulter), and incubated for 20 minutes at 4° C. In order to remove the red blood cells, the tubes are treated with Q-PREP Workstation and IMMUNOPREP Reagent System (Beckman Coulter). The cells are subsequently studied by flow cytometry (FC500, Beckman Coulter).

The depletion of NF-3C8 cells was quantified by using flow cytometry, based on the percentage of CD123+CD56+ cells compared to the percentage of CD3+ cells, which are used as reference cells.

The results are presented as a percentage of depletion of NF-3C8 cells. The control antibody corresponds to 0% depletion.

The depletion percentage was calculated as follows:
1^{st} Step: % of NF-3C8 vs CD3:

X=(number of NF-3C8 CD56+CD123+)/(number of CD3+ cells)*100
2^{nd} Step: % of depletion:

100−(X of “Ch 122A2 mAb”/X of “control”)*100

Results

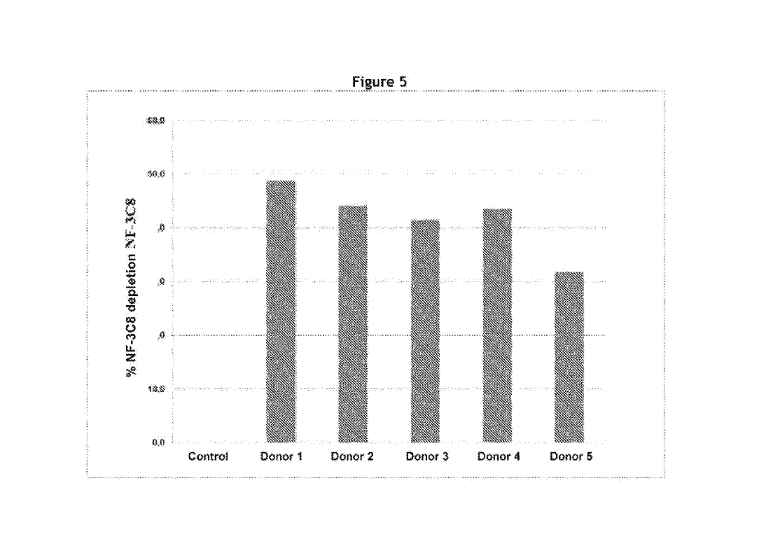

The results of the whole blood tests are presented in FIG. 5 and in Table 18 below.

TABLE 18
Data corresponding to FIG. 5

| | 16 hr incubation at 37° C. | | | |
|---|---|---|---|---|
| | Number of NF- | Number | % = | % |
| mAb = | 3C8 cells | of CD3+ | (CD56+C123+/ | depletion |
| 1 μg/ml | (CD56+CD123+) | cells | CD3+) * 100 | NF-3C8 |
| 1825 | | | | |
| 15 865 | | | | |
| Donor 1 | | | | |
| Control | 4708 | 16042 | 29.35 | 0.0 |
| Ch. | 2084 | 13860 | 15.04 | 48.8 |
| 122A2 | | | | |
| mAb | | | | |
| 1825 | | | | |
| 15 904 | | | | |
| Donor 2 | | | | |
| Control | 4186 | 13703 | 30.55 | 0.0 |
| Ch. | 2246 | 13177 | 17.04 | 44.2 |
| 122A2 | | | | |
| mAb | | | | |
| 1825 | | | | |
| 15 904 | | | | |
| Donor 3 | | | | |
| Control | 3842 | 3739 | 102.75 | 0.0 |
| Ch. | 2140 | 3560 | 60.11 | 41.5 |
| 122A2 | | | | |
| mAb | | | | |
| 1525 | | | | |
| 15 905 | | | | |
| Donor 4 | | | | |
| Control | 3261 | 8384 | 38.90 | 0.0 |
| Ch. | 1983 | 9035 | 21.95 | 43.6 |
| 122A2 | | | | |
| mAb | | | | |
| 1825 | | | | |
| 15 905 | | | | |
| Donor 5 | | | | |
| Control | 2766 | 10915 | 25.34 | 0.0 |
| Ch. | 2843 | 16459 | 17.27 | 31.8 |
| 122A2 | | | | |
| mAb | | | | |

These results show that after incubation for 16 hours at 37° C. the antibody “Ch. 122A2 mAb” induces depletion of NF-3C8 cells in the blood samples from five healthy donors independent of each other. In contrast, the control antibody does not induce depletion.

These results thus show that the antibody 122A2 is capable of inducing the depletion of NF-3C8 cells.

### Example 4

### Impact of Elimination of pDC on the ADCC Activity and Phagocytosis of an Antibody Directed Against a Tumour Antigen

Principle of the Study

The pDCs (plasmacytoid dendritic cells) are responsible for the differentiation of regulatory T cells inter alia by ICOS/ICOSL interaction. The regulatory T cells thus differentiated exert mechanisms of immunosuppression on the functions of other cells of the immune system, in particular NK cells, via cell/cell contact but also via the secretion of immunomodulating cytokines such as IL-10, IL-35 and TGF-β.

By means of a cascade effect, it is thus possible to study the impact of elimination of pDCs on the activity of an anti-cancer agent specific to a tumour involving pDC activation in situ. In particular, the protective effect of the anti-CD303 antibodies targeting the pDCs may be demonstrated by the correlative study of the reduction or elimination of the cytokines IL-10 and TGF-β in the tumour environment and its impact on the effector functions of an anti-cancer agent administered, which is specific to the tumour (for example anti-solid tumour antigen antibody). Indeed, according to this model, the anti-CD303 antibodies that deplete the pDCs lead to the limiting of the immunosuppressive effects of regulatory T cells, thereby limiting their secretion of IL-10 and TGF-β. This limiting of secretion of IL-10 and TGF-β correlates with improved ADCC of NK cells, thus validating the indirect stimulatory effect of anti-CD303 antibodies on the anticancer action of the anti-tumour antigen antibody, for example, anti-solid tumour antigen antibody.

1—Effect of IL-10 and TGF-β on the ADCC Activity of an Anti-Tumour Antigen Antibody

In order to test the ADCC in a tumour context involving activation of pDCs on one hand or by eliminating the pDCs on the other hand, the tumour antigen presenting cells (35000 cells/well) are incubated for 16 hours at 37° C., in a 96-well flat-bottom plate with effector NK cells and increasing concentrations of the tumour antigen-specific antibodies, in the presence or absence of: IL-10 (5 and 50 ng/mL), TGF-β (5 and 50 ng/mL) or IL-10+TGF-β (5 and 50 ng/mL for each of the cytokines). After incubation, the supernatant is collected.

Lysis of the target cells induced by the anti-cancer antibodies is measured chromogenically by quantifying the intracellular lactate dehydrogenase (LDH) enzyme released into the supernatant by the lysed target cells (Cytotoxicity Detection Kit (LDH), Roche Diagnostics).

The percentage of lysis is calculated according to the following formula:

% lysis=[(ER−SR)/(100−SR)]−[(NC−SR)/(100−SR)]

Where ER and SR represent the experimental release (ER) and the spontaneous release (SR) of LDH, respectively, and NC represents the natural cytotoxicity of the NK cells.

The results (% lysis) are expressed as a function of antibody dilution factor. For each antibody, the “50% activity” value corresponds to the antibody dilution factor necessary to induce 50% of the plateau value obtained for this antibody. This value may be calculated with the PRISM software.

Conclusion: By means of a cascade effect, comparison of the percentage of lysis observable in the absence of cytokines IL-10 and/or TGF-β, with the percentage of lysis observable in the presence of these same cytokines, makes it possible to evaluate the impact of the depletion of pDCs present on the tumour site. It may thus be demonstrated that the anti-CD303 antibodies enable indirectly potentiating the effect of the anti-cancer antibodies.

2—Effect of IL-10 and TGF-β on Phagocytosis Induced by an Anti-Tumour Antigen Antibody

The monocytes are differentiated into CD16+ macrophages (M2 like) over 2 days in RPMI 1640+10% FBS (fetal bovine serum)+M-CSF 50 ng/mL for 48 h.

The cells expressing the tumour antigen and the macrophages are labelled with PKH-67 (green fluorescent) and PKH-26 (red fluorescent), respectively.

The cells expressing the tumour antigen are opsonised with 10 μg/mL of the antibody specific to this antigen or with an irrelevant antibody and then incubated with the macrophages (1,105 of each cells/wells) in the absence or presence of different concentrations of IL-10 (5 and 50 ng/mL) alone, of TGF-β (5 and 50 ng/mL) alone, and of IL-10+TGF-β (5 and 50 ng/mL).

After 3 h of incubation at 37° C., the cells are placed on a counting chamber (Mallassez) and observed with a fluorescence microscope.

The percentage of phagocytosis is evaluated by counting the number of macrophages (at least 100 macrophages) containing tumour cells.

Conclusion: By means of a cascade effect, comparison of the percentage of phagocytosis observable in the absence of cytokines IL-10 and/or TGF-β, with the percentage of lysis observable in the presence of these same cytokines, makes it possible to evaluate the impact of the depletion of pDCs present on the tumour site. It may thus be demonstrated that the anti-CD303 antibodies enable indirectly potentiating the effect of the anti-cancer antibodies.

### Example 5

### Effect of an Anti-CD303 Antibody on the Activation of Regulatory T Cells (Treg)

1—Role of Anti-CD303 on the Phenotype and Expansion of Treg Cells in PBMC

Mononuclear cells (PBMC) are isolated from a tube of blood collected on anti-coagulant. The Treg cells are identified and phenotypically characterised by flow cytometry on the basis of 3 markers: CD4, CD25, and Fox P3.

Various differentamounts of the anti-CD303 antibody (from 1 ng to 10 μg/mL) are added to the PBMCs in the presence of IL-2 (500 U/ml). The number of Treg and the phenotype thereof are monitored over time (1 to 4 days).

Under the same conditions, beads coated with anti-CD3/anti-CD28 to stimulate T cell proliferation are added in a Treg/beads ratio of 4/1 in order to check and verify the activation of Tregs.

Conclusion: It may thus be demonstrated that the anti-CD303 antibody, in the absence of pDCs, have no direct impact on the expansion and immunosuppressive phenotype of regulatory T cells.

2—Role of Anti-CD303 on the Phenotype and Expansion of Treg Cells Purified in the Presence of pDC

Treg cells (CD4⁺, CD25⁺) were purified from PBMC by using a method in two steps: depletion of the CD4 negative cells (cells positive for the markers CD8, CD14, CD15, CD16, CD19, CD36, CD56, CD123, TCRγ/δ, and CD235a) and followed by positive selection of CD25⁺ cells.

Purified pDCs or pDC lines, eg obtained according to the method described in Maeda T et al., Int J Hematol. 2005 February; 81(2):148-54 (such as the CAL-1 or NF-3C8 line) are added in a Treg/pDC ratio of 100, 10 and 1. Various different amounts of the anti-CD303 antibody (from 1 ng to 10 μg/mL) are added to the Treg/pDC mixture in the presence of IL-2 (500 U/ml). The number of Treg and the phenotype thereof are monitored over time (1 to 4 days).

Under the same conditions, beads coated with anti-CD3/anti-CD28 to stimulate T cell proliferation are added in a Treg/beads ratio of 4/1 in order to check and verify the activation of Tregs.

A negative control in the absence of pDCs is established in a manner so as to verify the impact (neutral expected) of anti-CD303 directly on the Tregs.

Conclusion: Observation over several days of the expansion and differentiation of purified Treg cells in the presence of pDCs, after anti-CD303 antibody administration may provide the means to show that anti-CD303 administration is effective in reducing or suppressing/eliminating the immunosuppressive properties of pDCs.

### Example 6

### Depletion of Human pDCs via an Anti-CD303 Antibody In Vivo in the Treatment of a Solid Cancer

1—Generating a Model for Reproducible Study of a Pathological Situation in Humans

In order to study the effect of an anti-CD303 antibody in the treatment therapy of a solid cancer, a ‘humanised tumour mouse model’ (HTM) may be used. It is characterised by the development of a mature human immune system and the growth of human solid cancer cells that have previously been co-transplanted with the human hematopoietic stem cells.

This model advantageously makes it possible to bring together a number of elements that are relevant to the reproducibility of in vivo conditions: presence of human pDCs which alone express on their surface the target CD303, presence of infiltration of human Treg cells, presence of human tumour cells expressing at their surface a target tumour antigen, molecule targeted by an anti-tumour antibody and an immunocompetent murine host (NK type effector cells for ADCC activity).

Briefly, NOD-scid IL2Rynull mice (NSG) may be obtained, for example from Laboratoires Jackson, and housed in a pathogen-free specialised establishment. The newborns would be irradiated (1 Gy) during their first 48 hours of life and 3 hours thereafter undergo transplant by intrahepatic injection of 2.5×10⁵ human CD34+ cells isolated from umbilical cord blood (CB) in the presence of 3×10⁶ tumour cells specifically expressing a solid tumour antigen and expressing luciferase for bioluminescence monitoring.

2—Method that may be Used for Testing the Activity of Anti-CD303 Antibodies

Upon the tumour becoming visible by bioluminescence (IVIS), the HTM mice are treated with 20 mg/kg of anti-tumour antigen antibody via the peritoneal route every week and with the anti-CD303 antibody at a dose of 30 mg/kg every 3 days intravenously.

Monitoring of the effectiveness of the treatment is performed by means of bioluminescence monitoring; the survival of the animals as well as the survival rate in the absence of tumours are monitored. Blood samples are taken during the course of the study to ensure the effectiveness of the anti-CD303 antibody on the depletion of pDCs in this tumour context.

The different conditions tested are thus advantageously the following (10 animals per group):

1. HTM+anti-tumour antigen

2. HTM+anti-CD303

3. HTM+anti-tumour antigen+anti-CD303

4. HTM+Control isotype

Conclusion:

The murine model, HTM, may advantageously be used to evaluate the indirect effect of administration of an anti-CD303 antibody on the effect of the anti-solid tumour agent, the anti-tumour antigen antibody, under conditions that reproduce a physiological situation in vivo, in particular by comparing the results obtained under condition 3 above, with on the one hand those obtained under condition 1, and on the other hand those obtained under condition 2.

This model is thus useful for being able to evaluate the benefit, advantageously the synergistic effect, of administering an anti-CD303 antibody in combination with administration of an anti-tumour antigen antibody in a solid tumour.

1-13. (canceled)

14. A method for preventing or treating a tumour involving activation of plasmacytoid dendritic cells in the microenvironment of the said tumour in a patient in need thereof, the said plasmacytoid dendritic cells not being responsible for causing the tumour, comprising administering to said patient an antibody, in particular monoclonal or polyclonal, directed against the CD303 protein.

15. The method according to claim 14, wherein the plasmacytoid dendritic cells have immunosuppressive and/or tolerogenic properties.

16. The method according to claim 14, wherein the tumours involving activation of plasmacytoid dendritic cells are solid tumour or hematopoietic tumour.

17. The method according to claim 14, wherein the said antibody is selected from among a murine antibody, a chimeric antibody, a humanised antibody or a human antibody.

18. The method according to claim 14, wherein the antibody has a low fucose content that is less than or equal to 65%, and/or an oligomannose-type N-glycans content that is greater than or equal to 30%, and/or a galactose content that is greater than or equal to 50%.

19. The method according to claim 14, wherein the antibody is an antibody fragment selected from Fab, F(ab′)2, Fd, scFv, scFv dimer, diabody, triabody or tetrabody.

20. The method according to claim 14, wherein the said antibody or said fragment is conjugated with a bioactive molecule selected from among the following:
radioactive isotopes, in particular selected from At211, I131, I125, Y90, Re186, Re188, Sm153, Bi212, P32,
non-radioactive metals,
toxins, in particular selected from ricin, abrin, diphtheria toxin,
nucleic acids, in particular selected from antisense RNAs,
enzymes, in particular selected from RNases, biotin, avidin or streptavidin,
cytotoxic agents, in particular selected from among:
antifolates, and more particularly methotrexate, pemetrexed, raltitrexed;
anti-purines, and more particularly cladribine, fludarabine, azathioprine, azathioprine, mercaptopurine, 5-fluorouracil, capecitabine, cytarabine, gemcitabine,
topoisomerase I and II inhibitors,
alkylating agents and related agents, and more particularly chlormethine, cyclophosphamide, ifosfamide, carmustine, fotemustine, mitomycin C, cisplatin, carboplatin, oxaliplatin,
intercalating agents,
anthracyclines, more particularly selected from daunorubicin, doxorubicin and hydrochloride, epirubicin, idarubicin, bleomycin,
taxanes,
specific inhibitors of tyrosine kinase, imatinib, erlotinib.

21. The method according to claims 14, wherein the said antibody or said fragment is used in combination with at least one anti-cancer agent, in particular a chemical anti-cancer agent and/or an anti-cancer agent used in immunotherapy.

22. The method according to claim 21, wherein the said chemical anti-cancer agent is selected from among anti-metabolic agents, alkylating agents, intercalating agents, or molecules having an action on the mitotic spindle, and preferably:
the said metabolic agents are selected from among the following:
antifolates, in particular methotrexate, raltitrexed and pemetrexed,
anti-purines, in particular mercaptopurine, thioguanine, pentostatin, cladribine and fludarabine,
anti-pyrimidines, in particular 5-fluorouracil, tegafur uracil, cytarabine and capecitabine, and
anti-metabolics, in particular hydroxycarbamide, hydroxyurea and gemcitabine,
the said alkylating agents are selected from among the following:
nitrogen mustards, in particular chlorambucil, melphelan, chlormethine, metachloroethamine, estramustine, ifosfamide and cyclophosphamide,
nitrosoureas, in particular fotemustine, lomustine, carmustine, streptozocin,
organoplatines, in particular carboplatin, cisplatin and oxaliplatin,
ethylene imines, in particular thiotepa and altretamine,
triazenes, in particular procarbazine, temozolomide and dacarbazine,
alkylating agents, in particular busulfan, mitomycin C and pipobroman,
the said intercalating agents are selected from among the following:
camptothecin derivatives, in particular irinotecan and topotecan
antrhracyclines, in particular epirubicin, daunorubicin, doxorubicin, pirarubicin and idarubicin,
intercalating agents, in particular mitoxantrone, amsacrine, elliptinium, actinomycin D, dactinomycin, etoposide, and bleomycin;
the said molecules having an action on the mitotic spindle are selected from among the following:
vinca alkaloids or spindle poisons, in particular vinorelbine, vindesine, vincristine and vinblastine,
taxoids or spindle microtubule stabilising agents, in particular paclitaxel and docetaxel,
tyrosine kinase inhibitors, in particular dasatinib, erlotinib, imatinib, sorafenib and sunitinib.

23. The method according to claim 21, wherein the said anti-cancer agent used in immunotherapy is a targeted tumour specific antibody, anti-CD123 antibody, or a TLR agonist.

24. The method according to claim 21, wherein the said use of the said antibody or said fragment and the said use of the said anti-cancer agent are simultaneous, separate or spread out over time.

25. The method according to claim 21, wherein it is coupled with radiotherapy.

26. The method according to claim 14, wherein the said prevention or the said treatment is effected in a patient with depletion of plasmacytoid dendritic cells, or in a patient in need of or requiring a depletion of plasmacytoid dendritic cells.

27. The method of claim 16, wherein the tumors involving activation of plasmacytoid dendritic cells are solid tumors involving infiltration of plasmacytoid dendritic cells into the microenvironment of the said tumor.

28. The method of claim 16, wherein the tumors involving activation of plasmacytoid dendritic cells are hematopoietic tumors belonging to the group consisting of multiple myeloma, lymphoma, and leukemia.

29. The method of claim 17, wherein the said antibody is a chimeric antibody selected from a murine/human chimeric antibody or a human-macaque chimeric antibody.

30. The method according to claims 19, wherein the said antibody or said fragment is used in combination with at least one anti-cancer agent, in particular a chemical anti-cancer agent and/or an anti-cancer agent used in immunotherapy.

31. The method according to claim 22, wherein the said use of the said antibody or said fragment and the said use of the said anti-cancer agent are simultaneous, separate or spread out over time


SEQUENCE LISTING (130 sequences)

SEQ ID NO: 1 - protein, 8 aa, artificial
  CDR1-H-famille 1
GYTFTDYS

SEQ ID NO: 2 - protein, 8 aa, artificial
  CDR2-H-famille 1
  misc_feature (3)..(3): Xaa can be any naturally occurring amino acid
  misc_feature (8)..(8): Xaa can be any naturally occurring amino acid
ISXYYGDX

SEQ ID NO: 3 - protein, 11 aa, artificial
  CDR3-H-famille 1
  misc_feature (4)..(6): Xaa can be any naturally occurring amino acid
  misc_feature (8)..(10): Xaa can be any naturally occurring amino acid
ARNXXXYXXXY

SEQ ID NO: 4 - protein, 6 aa, artificial
  CDR1-L-famille 1
  misc_feature (4)..(4): Xaa can be any naturally occurring amino acid
QDIXNY

SEQ ID NO: 5 - protein, 3 aa, artificial
  CDR2-L-famille 1
YTS

SEQ ID NO: 6 - protein, 9 aa, artificial
  CDR3-L-famille 1
  misc_feature (4)..(4): Xaa can be any naturally occurring amino acid
QQGXTLPWT

SEQ ID NO: 7 - protein, 8 aa, artificial
  CDR1-H-famille 2
  misc_feature (7)..(7): Xaa can be any naturally occurring amino acid
GYTFTDXS

SEQ ID NO: 8 - protein, 8 aa, artificial
  CDR2-H-famille 2
  misc_feature (7)..(7): Xaa can be any naturally occurring amino acid
INTETGXP

SEQ ID NO: 9 - protein, 14 aa, artificial
  CDR3-H-famille 2
  misc_feature (1)..(1): Xaa can be any naturally occurring amino acid
  misc_feature (12)..(12): Xaa can be any naturally occurring amino acid
XRNGYYVGYYAXDY

SEQ ID NO: 10 - protein, 5 aa, artificial
  CDR1-L-famille 2
  misc_feature (4)..(4): Xaa can be any naturally occurring amino acid
SSVXY

SEQ ID NO: 11 - protein, 3 aa, artificial
  CDR2-L-famille 2
STS

SEQ ID NO: 12 - protein, 9 aa, artificial
  CDR3-L-famille 2
  misc_feature (8)..(8): Xaa can be any naturally occurring amino acid
QQRRSYPXT

SEQ ID NO: 13 - protein, 8 aa, artificial
  CDR1-H-122A2
GYTFTDYS

SEQ ID NO: 14 - protein, 8 aa, artificial
  CDR2-H-122A2
ISTYYGDS

SEQ ID NO: 15 - protein, 11 aa, artificial
  CDR3-H-122A2
ARNGNFYVMDY

SEQ ID NO: 16 - protein, 6 aa, artificial
  CDR1-L-122A2
QDISNY

SEQ ID NO: 17 - protein, 3 aa, artificial
  CDR2-L-122A2
YTS

SEQ ID NO: 18 - protein, 9 aa, artificial
  CDR3-L-122A2
QQGNTLPWT

SEQ ID NO: 19 - protein, 8 aa, artificial
  CDR1-H-102E9
GYTFTDYS

SEQ ID NO: 20 - protein, 8 aa, artificial
  CDR2-H-102E9
INTETGEP

SEQ ID NO: 21 - protein, 14 aa, artificial
  CDR3-H-102E9
TRNGYYVGYYAMDY

SEQ ID NO: 22 - protein, 5 aa, artificial
  CDR1-L-102E9
SSVIY

SEQ ID NO: 23 - protein, 3 aa, artificial
  CDR2-L-102E9
STS

SEQ ID NO: 24 - protein, 9 aa, artificial
  CDR3-L-102E9
QQRRSYPFT

SEQ ID NO: 25 - protein, 8 aa, artificial
  CDR1-H-104C12
GYTFTDYS

SEQ ID NO: 26 - protein, 8 aa, artificial
  CDR2-H-104C12
ISPYYGDT

SEQ ID NO: 27 - protein, 11 aa, artificial
  CDR3-H-104C12
ARNDDYYRFAY

SEQ ID NO: 28 - protein, 6 aa, artificial
  CDR1-L-104C12
QDINNY

SEQ ID NO: 29 - protein, 3 aa, artificial
  CDR2-L-104C12
YTS

SEQ ID NO: 30 - protein, 9 aa, artificial
  CDR3-L-104C12
QQGKTLPWT

SEQ ID NO: 31 - protein, 8 aa, artificial
  CDR1-H-114D11
GYTFTDSS

SEQ ID NO: 32 - protein, 8 aa, artificial
  CDR2-H-114D11
INTETGGP

SEQ ID NO: 33 - protein, 14 aa, artificial
  CDR3-H-114D11
ARNGYYVGYYALDY

SEQ ID NO: 34 - protein, 5 aa, artificial
  CDR1-L-114D11
SSVFY

SEQ ID NO: 35 - protein, 3 aa, artificial
  CDR2-L-114D11
STS

SEQ ID NO: 36 - protein, 9 aa, artificial
  CDR3-L-114D11
QQRRSYPYT

SEQ ID NO: 37 - protein, 8 aa, artificial
  CDR1-H-104E10
GYTFTDYS

SEQ ID NO: 38 - protein, 8 aa, artificial
  CDR2-H-104E10
INTETGEP

SEQ ID NO: 39 - protein, 14 aa, artificial
  CDR3-H-104E10
ARNGYYVGYYAMDY

SEQ ID NO: 40 - protein, 5 aa, artificial
  CDR1-L-104E10
SSVIY

SEQ ID NO: 41 - protein, 3 aa, artificial
  CDR2-L-104E10
STS

SEQ ID NO: 42 - protein, 9 aa, artificial
  CDR3-L-104E10
QQRRSYPYT

SEQ ID NO: 43 - protein, 118 aa, artificial
  VH-122A2
QVQLQQSGAELVRPGVSVKISCKGSGYTFTDYSMHWVKQSHAKSLEWIGVISTYYGDSNYNQKFKGKATMTVDKSSTTAYMELARLTSEDSAIYYCARNGNFYVMDYWGQGTSVTVSS

SEQ ID NO: 44 - protein, 121 aa, artificial
  VH-102E9
QIHLVQSGPDLKKPGETVKISCKASGYTFTDYSMHWVKQAPGKGLKWMGWINTETGEPTYADDFKGRFAFSLESSASTAFLQINNLKNEDTSTYFCTRNGYYVGYYAMDYWGQGTSVTVSS

SEQ ID NO: 45 - protein, 118 aa, artificial
  VH-104C12
QVQLQQSGAELVGPGVSVKISCKGSGYTFTDYSMHWVKQSHAKSLEWIGVISPYYGDTNYNQKFKGKATMTVDKSSSTAYMELASLTSEDSAIYFCARNDDYYRFAYWGQGTLVTVSA

SEQ ID NO: 46 - protein, 121 aa, artificial
  VH-114D11
QIQLVQSGPELKKPGETVKISCKASGYTFTDSSMHWVQQAPNKGLKWMGWINTETGGPTYADDFKGRFAFSLETSARTAYLQINNLKNEDTATYFCARNGYYVGYYALDYWGQGTSVTVSS

SEQ ID NO: 47 - protein, 121 aa, artificial
  VH-104E10
QIQLVQSGPELKKPGETVKISCKASGYTFTDYSMHWVKQAPGKGLKWMGWINTETGEPTYADDFKGRFAFSLETSATTAYLQINNFKNEDTATYFCARNGYYVGYYAMDYWGQGTSVTVSS

SEQ ID NO: 48 - protein, 107 aa, artificial
  VL-122A2
DIQMTQTTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIK

SEQ ID NO: 49 - protein, 106 aa, artificial
  VL-102E9
QIVLTQSPAIMSASPGEKVTITCSASSSVIYIHWFQQKPGTSPKLWIYSTSYLASGVPARFSGSGSGTSYSLTISRMEAEDAATYYCQQRRSYPFTFGGGTKLEIK

SEQ ID NO: 50 - protein, 107 aa, artificial
  VL-104C12
DLQMTQTPSSLSASLGDRVTISCRASQDINNYLSWYQEKPDGTFKLLIYYTSRLHSGVPSRFSGSGSGTDYSLTVRNLEQEDIGTYFCQQGKTLPWTFGGGTKLEIR

SEQ ID NO: 51 - protein, 106 aa, artificial
  VL-114D11
QIVLTQSPAIMSASPGEKVTITCSASSSVFYMHWFQQKPGTSPKLWIYSTSNLASGVPARFSGSGSGTSYSLTISRMEAEDAATYYCQQRRSYPYTFGGGTKLEIK

SEQ ID NO: 52 - protein, 106 aa, artificial
  VL-104E10
QIVLTQSPAIMSASPGEKVTMTCSASSSVIYMHWFQQKPGTSPKLWIYSTSNLASGVPARFSGSGSGTSYSLTISRMEAEDAATYYCQQRRSYPYTFGGGTKLEIK

SEQ ID NO: 53 - protein, 329 aa, artificial
  CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 107 aa, artificial [sequence omitted]

SEQ ID NO: 55 - protein, 447 aa, artificial
  VH-CH-122A2
QVQLQQSGAELVRPGVSVKISCKGSGYTFTDYSMHWVKQSHAKSLEWIGVISTYYGDSNYNQKFKGKATMTVDKSSTTAYMELARLTSEDSAIYYCARNGNFYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 450 aa, artificial
  VH-CH-102E9
QIHLVQSGPDLKKPGETVKISCKASGYTFTDYSMHWVKQAPGKGLKWMGWINTETGEPTYADDFKGRFAFSLESSASTAFLQINNLKNEDTSTYFCTRNGYYVGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 447 aa, artificial
  VH-CH-104C12
QVQLQQSGAELVGPGVSVKISCKGSGYTFTDYSMHWVKQSHAKSLEWIGVISPYYGDTNYNQKFKGKATMTVDKSSSTAYMELASLTSEDSAIYFCARNDDYYRFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 450 aa, artificial
  VH-CH-114D11
QIQLVQSGPELKKPGETVKISCKASGYTFTDSSMHWVQQAPNKGLKWMGWINTETGGPTYADDFKGRFAFSLETSARTAYLQINNLKNEDTATYFCARNGYYVGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 450 aa, artificial
  VH-CH-104E10
QIQLVQSGPELKKPGETVKISCKASGYTFTDYSMHWVKQAPGKGLKWMGWINTETGEPTYADDFKGRFAFSLETSATTAYLQINNFKNEDTATYFCARNGYYVGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 214 aa, artificial
  VL-CL-122A2
DIQMTQTTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 213 aa, artificial
  VL-CL-102E9
QIVLTQSPAIMSASPGEKVTITCSASSSVIYIHWFQQKPGTSPKLWIYSTSYLASGVPARFSGSGSGTSYSLTISRMEAEDAATYYCQQRRSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 214 aa, artificial
  VL-CL-104C12
DLQMTQTPSSLSASLGDRVTISCRASQDINNYLSWYQEKPDGTFKLLIYYTSRLHSGVPSRFSGSGSGTDYSLTVRNLEQEDIGTYFCQQGKTLPWTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 213 aa, artificial
  VL-CL-114D11
QIVLTQSPAIMSASPGEKVTITCSASSSVFYMHWFQQKPGTSPKLWIYSTSNLASGVPARFSGSGSGTSYSLTISRMEAEDAATYYCQQRRSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 213 aa, artificial
  VL-CL-104E10
QIVLTQSPAIMSASPGEKVTMTCSASSSVIYMHWFQQKPGTSPKLWIYSTSNLASGVPARFSGSGSGTSYSLTISRMEAEDAATYYCQQRRSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 18 aa, artificial
  MB7 signal peptide
MRWSWIFLLLLSITSANA

SEQ ID NO: 66 - protein, 136 aa, artificial
  MB7-VH-122A2
MRWSWIFLLLLSITSANAQVQLQQSGAELVRPGVSVKISCKGSGYTFTDYSMHWVKQSHAKSLEWIGVISTYYGDSNYNQKFKGKATMTVDKSSTTAYMELARLTSEDSAIYYCARNGNFYVMDYWGQGTSVTVSS

SEQ ID NO: 67 - protein, 139 aa, artificial
  MB7-VH-102E9
MRWSWIFLLLLSITSANAQIHLVQSGPDLKKPGETVKISCKASGYTFTDYSMHWVKQAPGKGLKWMGWINTETGEPTYADDFKGRFAFSLESSASTAFLQINNLKNEDTSTYFCTRNGYYVGYYAMDYWGQGTSVTVSS

SEQ ID NO: 68 - protein, 136 aa, artificial
  MB7-VH-104C12
MRWSWIFLLLLSITSANAQVQLQQSGAELVGPGVSVKISCKGSGYTFTDYSMHWVKQSHAKSLEWIGVISPYYGDTNYNQKFKGKATMTVDKSSSTAYMELASLTSEDSAIYFCARNDDYYRFAYWGQGTLVTVSA

SEQ ID NO: 69 - protein, 139 aa, artificial
  MB7-VH-114D11
MRWSWIFLLLLSITSANAQIQLVQSGPELKKPGETVKISCKASGYTFTDSSMHWVQQAPNKGLKWMGWINTETGGPTYADDFKGRFAFSLETSARTAYLQINNLKNEDTATYFCARNGYYVGYYALDYWGQGTSVTVSS

SEQ ID NO: 70 - protein, 139 aa, artificial
  MB7-VH-104E10
MRWSWIFLLLLSITSANAQIQLVQSGPELKKPGETVKISCKASGYTFTDYSMHWVKQAPGKGLKWMGWINTETGEPTYADDFKGRFAFSLETSATTAYLQINNFKNEDTATYFCARNGYYVGYYAMDYWGQGTSVTVSS

SEQ ID NO: 71 - protein, 125 aa, artificial
  MB7-VL-122A2
MRWSWIFLLLLSITSANADIQMTQTTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIK

SEQ ID NO: 72 - protein, 124 aa, artificial
  MB7-VL-102E9
MRWSWIFLLLLSITSANAQIVLTQSPAIMSASPGEKVTITCSASSSVIYIHWFQQKPGTSPKLWIYSTSYLASGVPARFSGSGSGTSYSLTISRMEAEDAATYYCQQRRSYPFTFGGGTKLEIK

SEQ ID NO: 73 - protein, 125 aa, artificial
  MB7-VL-104C12
MRWSWIFLLLLSITSANADLQMTQTPSSLSASLGDRVTISCRASQDINNYLSWYQEKPDGTFKLLIYYTSRLHSGVPSRFSGSGSGTDYSLTVRNLEQEDIGTYFCQQGKTLPWTFGGGTKLEIR

SEQ ID NO: 74 - protein, 124 aa, artificial
  MB7-VL-114D11
MRWSWIFLLLLSITSANAQIVLTQSPAIMSASPGEKVTITCSASSSVFYMHWFQQKPGTSPKLWIYSTSNLASGVPARFSGSGSGTSYSLTISRMEAEDAATYYCQQRRSYPYTFGGGTKLEIK

SEQ ID NO: 75 - protein, 124 aa, artificial
  MB7-VL-104E10
MRWSWIFLLLLSITSANAQIVLTQSPAIMSASPGEKVTMTCSASSSVIYMHWFQQKPGTSPKLWIYSTSNLASGVPARFSGSGSGTSYSLTISRMEAEDAATYYCQQRRSYPYTFGGGTKLEIK

SEQ ID NO: 76 - protein, 465 aa, artificial
  MB7-VH-CH-122A2
MRWSWIFLLLLSITSANAQVQLQQSGAELVRPGVSVKISCKGSGYTFTDYSMHWVKQSHAKSLEWIGVISTYYGDSNYNQKFKGKATMTVDKSSTTAYMELARLTSEDSAIYYCARNGNFYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 468 aa, artificial
  MB7-VH-CH-102E9
MRWSWIFLLLLSITSANAQIHLVQSGPDLKKPGETVKISCKASGYTFTDYSMHWVKQAPGKGLKWMGWINTETGEPTYADDFKGRFAFSLESSASTAFLQINNLKNEDTSTYFCTRNGYYVGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 465 aa, artificial
  MB7-VH-CH-104C12
MRWSWIFLLLLSITSANAQVQLQQSGAELVGPGVSVKISCKGSGYTFTDYSMHWVKQSHAKSLEWIGVISPYYGDTNYNQKFKGKATMTVDKSSSTAYMELASLTSEDSAIYFCARNDDYYRFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 468 aa, artificial
  MB7-VH-CH-114D11
MRWSWIFLLLLSITSANAQIQLVQSGPELKKPGETVKISCKASGYTFTDSSMHWVQQAPNKGLKWMGWINTETGGPTYADDFKGRFAFSLETSARTAYLQINNLKNEDTATYFCARNGYYVGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 468 aa, artificial
  MB7-VH-CH-104E10
MRWSWIFLLLLSITSANAQIQLVQSGPELKKPGETVKISCKASGYTFTDYSMHWVKQAPGKGLKWMGWINTETGEPTYADDFKGRFAFSLETSATTAYLQINNFKNEDTATYFCARNGYYVGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 232 aa, artificial
  MB7-VL-CL-122A2
MRWSWIFLLLLSITSANADIQMTQTTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 231 aa, artificial
  MB7-VL-CL-102E9
MRWSWIFLLLLSITSANAQIVLTQSPAIMSASPGEKVTITCSASSSVIYIHWFQQKPGTSPKLWIYSTSYLASGVPARFSGSGSGTSYSLTISRMEAEDAATYYCQQRRSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 232 aa, artificial
  MB7-VL-CL-104C12
MRWSWIFLLLLSITSANADLQMTQTPSSLSASLGDRVTISCRASQDINNYLSWYQEKPDGTFKLLIYYTSRLHSGVPSRFSGSGSGTDYSLTVRNLEQEDIGTYFCQQGKTLPWTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 231 aa, artificial
  MB7-VL-CL-114D11
MRWSWIFLLLLSITSANAQIVLTQSPAIMSASPGEKVTITCSASSSVFYMHWFQQKPGTSPKLWIYSTSNLASGVPARFSGSGSGTSYSLTISRMEAEDAATYYCQQRRSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 231 aa, artificial
  MB7-VL-CL-104E10
MRWSWIFLLLLSITSANAQIVLTQSPAIMSASPGEKVTMTCSASSSVIYMHWFQQKPGTSPKLWIYSTSNLASGVPARFSGSGSGTSYSLTISRMEAEDAATYYCQQRRSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - DNA, 354 bp, artificial
  VH-122A2
caggtccagctgcagcagtctggggctgagctggtgaggcctggggtctcagtgaagatttcctgcaagggttctggctacacattcactgattattctatgcactgggtgaagcagagtcatgcaaagagtctagagtggattggagttattagtacttactatggtgattctaactataaccagaagttcaagggcaaggccacaatgactgtagacaaatcctccaccacagcctatatggaacttgccagactgacatctgaggattctgccatctattactgtgcaagaaatggtaatttctatgttatggactactggggtcaaggaacctcagtcaccgtctcctca

SEQ ID NO: 87 - DNA, 363 bp, artificial
  VH-102E9
cagatccatttggtgcagtctggacctgacctgaagaagcctggagagacagtcaagatctcctgcaaggcttctggttataccttcacagactattcaatgcactgggtgaagcaggctccaggaaagggtttaaagtggatgggctggataaacactgagactggtgaaccaacatatgcagatgacttcaagggacggtttgccttctctttggaaagttctgccagcactgcctttttgcagatcaacaacctcaaaaatgaggacacgtctacatatttctgtactagaaatggttactacgtgggttactatgctatggactactggggtcaaggaacctcagtcaccgtctcctca

SEQ ID NO: 88 - DNA, 353 bp, artificial
  VH-104C12
caggtccagctgcagcagtctggggctgagctggtggggcctggggtctcagtgaagatttcctgcaagggttctggctacacattcactgattattctatgcactgggtaaagcagagtcatgcaaagagtctagagtggattggagttattagtccttactatggtgatactaactacaaccagaagttcaagggcaaggccacaatgactgtagacaaatcctccagcacagcctatatggaacttgccagtctgacatctgaggattctgccatctatttctgtgcaagaaatgatgattactacaggtttgcttactggggccaagggactctggtcactgtctctgc

SEQ ID NO: 89 - DNA, 363 bp, artificial
  VH-114D11
cagatccagttggtgcagtctggacctgagctgaagaagcctggagagacagtcaagatctcctgcaaggcttctggttataccttcacagactcttcaatgcactgggtgcagcaggctccaaacaagggtttaaagtggatgggctggataaacactgagactggtgggccaacgtatgcagatgatttcaagggacggtttgccttctctttggaaacctctgccagaactgcctatttgcagatcaacaacctcaaaaatgaggacacggctacatatttctgtgctagaaatggatactacgtggggtactatgctctggactactggggtcaaggaacctcagtcaccgtctcctca

SEQ ID NO: 90 - DNA, 363 bp, artificial
  VH-104E10
cagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaacactgagactggtgagccaacatatgcagatgacttcaagggacggtttgccttctctttggaaacctctgccaccactgcctatttgcagatcaacaacttcaaaaatgaggacacggctacatatttctgtgctagaaatggttactacgtgggatattatgctatggactactggggtcaaggaacctcagtcaccgtctcctca

SEQ ID NO: 91 - DNA, 321 bp, artificial
  VL-122A2
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggaccaagaagatattgccacttacttttgccaacagggtaatacgcttccttggacgttcggtggaggcaccaagctggaaatcaaa

SEQ ID NO: 92 - DNA, 318 bp, artificial
  VL-102E9
caaattgttctcacccagtctccagcaatcatgtctgcatctccaggggagaaggtcaccataacctgcagtgccagctcaagtgtaatttacattcactggttccagcagaagccaggcacttctcccaaactctggatttatagcacatcctacctggcttctggagtccctgctcgcttcagtggcagtggatctgggacctcttactctctcacaatcagccgaatggaggctgaagatgctgccacttattactgccagcagaggagaagttacccgttcacgttcggaggggggaccaagctggaaataaaa

SEQ ID NO: 93 - DNA, 320 bp, artificial
  VL-104C12
gatctccagatgacacagactccatcctccctgtctgcctctctgggagacagagtcaccatcagttgcagggcaagtcaggacattaacaattatttaagctggtatcaggagaaaccagatggaacttttaaactcctgatctactacacatcaagattacactcaggagtcccatcaaggttcagtggcagtgggtctggaacagattattctctcaccgttcgcaacctggaacaggaagatattggcacttacttttgccaacagggtaaaacgcttccgtggacgttcggtggaggcaccaagctggaaatcag

SEQ ID NO: 94 - DNA, 318 bp, artificial
  VL-114D11
caaattgttctcacccagtctccagcaatcatgtctgcatctccaggggagaaggtcaccataacctgcagtgccagctcaagtgtattttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagaagttacccgtacacgttcggaggggggaccaagctggaaataaaa

SEQ ID NO: 95 - DNA, 318 bp, artificial
  VL-104E10
caaattgttctcacccagtctccagcaatcatgtctgcatctccaggggagaaggtcaccatgacctgcagtgccagttcaagtgtaatttacatgcactggttccagcagaagccaggcacttctcccaaactctggatttatagcacatccaacctggcttctggagtccctgctcgcttcagtggcagtggatctgggacatcttactctctcacaatcagccgaatggaggctgaagatgctgccacttattactgccagcaaaggagaagttacccgtacacgttcggaggggggaccaagctggaaataaaa

SEQ ID NO: 96 - DNA, 990 bp, artificial
  CH
gcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 97 - DNA, 321 bp, artificial
  CL
cgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 98 - DNA, 1344 bp, artificial
  VH-CH-122A2
caggtccagctgcagcagtctggggctgagctggtgaggcctggggtctcagtgaagatttcctgcaagggttctggctacacattcactgattattctatgcactgggtgaagcagagtcatgcaaagagtctagagtggattggagttattagtacttactatggtgattctaactataaccagaagttcaagggcaaggccacaatgactgtagacaaatcctccaccacagcctatatggaacttgccagactgacatctgaggattctgccatctattactgtgcaagaaatggtaatttctatgttatggactactggggtcaaggaacctcagtcaccgtctcctcagcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 99 - DNA, 1353 bp, artificial
  VH-CH-102E9
cagatccatttggtgcagtctggacctgacctgaagaagcctggagagacagtcaagatctcctgcaaggcttctggttataccttcacagactattcaatgcactgggtgaagcaggctccaggaaagggtttaaagtggatgggctggataaacactgagactggtgaaccaacatatgcagatgacttcaagggacggtttgccttctctttggaaagttctgccagcactgcctttttgcagatcaacaacctcaaaaatgaggacacgtctacatatttctgtactagaaatggttactacgtgggttactatgctatggactactggggtcaaggaacctcagtcaccgtctcctcagcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 100 - DNA, 1343 bp, artificial
  VH-CH-104C12
caggtccagctgcagcagtctggggctgagctggtggggcctggggtctcagtgaagatttcctgcaagggttctggctacacattcactgattattctatgcactgggtaaagcagagtcatgcaaagagtctagagtggattggagttattagtccttactatggtgatactaactacaaccagaagttcaagggcaaggccacaatgactgtagacaaatcctccagcacagcctatatggaacttgccagtctgacatctgaggattctgccatctatttctgtgcaagaaatgatgattactacaggtttgcttactggggccaagggactctggtcactgtctctgcgcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 101 - DNA, 1353 bp, artificial
  VH-CH-114D11
cagatccagttggtgcagtctggacctgagctgaagaagcctggagagacagtcaagatctcctgcaaggcttctggttataccttcacagactcttcaatgcactgggtgcagcaggctccaaacaagggtttaaagtggatgggctggataaacactgagactggtgggccaacgtatgcagatgatttcaagggacggtttgccttctctttggaaacctctgccagaactgcctatttgcagatcaacaacctcaaaaatgaggacacggctacatatttctgtgctagaaatggatactacgtggggtactatgctctggactactggggtcaaggaacctcagtcaccgtctcctcagcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 102 - DNA, 1353 bp, artificial
  VH-CH-104E10
cagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaacactgagactggtgagccaacatatgcagatgacttcaagggacggtttgccttctctttggaaacctctgccaccactgcctatttgcagatcaacaacttcaaaaatgaggacacggctacatatttctgtgctagaaatggttactacgtgggatattatgctatggactactggggtcaaggaacctcagtcaccgtctcctcagcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 103 - DNA, 642 bp, artificial
  VL-CL-122A2
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggaccaagaagatattgccacttacttttgccaacagggtaatacgcttccttggacgttcggtggaggcaccaagctggaaatcaaacgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 104 - DNA, 639 bp, artificial
  VL-CL-102E9
caaattgttctcacccagtctccagcaatcatgtctgcatctccaggggagaaggtcaccataacctgcagtgccagctcaagtgtaatttacattcactggttccagcagaagccaggcacttctcccaaactctggatttatagcacatcctacctggcttctggagtccctgctcgcttcagtggcagtggatctgggacctcttactctctcacaatcagccgaatggaggctgaagatgctgccacttattactgccagcagaggagaagttacccgttcacgttcggaggggggaccaagctggaaataaaacgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 105 - DNA, 641 bp, artificial
  VL-CL-104C12
gatctccagatgacacagactccatcctccctgtctgcctctctgggagacagagtcaccatcagttgcagggcaagtcaggacattaacaattatttaagctggtatcaggagaaaccagatggaacttttaaactcctgatctactacacatcaagattacactcaggagtcccatcaaggttcagtggcagtgggtctggaacagattattctctcaccgttcgcaacctggaacaggaagatattggcacttacttttgccaacagggtaaaacgcttccgtggacgttcggtggaggcaccaagctggaaatcagcgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 106 - DNA, 639 bp, artificial
  VL-CL-114D11
caaattgttctcacccagtctccagcaatcatgtctgcatctccaggggagaaggtcaccataacctgcagtgccagctcaagtgtattttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagaagttacccgtacacgttcggaggggggaccaagctggaaataaaacgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 107 - DNA, 639 bp, artificial
  VL-CL-104E10
caaattgttctcacccagtctccagcaatcatgtctgcatctccaggggagaaggtcaccatgacctgcagtgccagttcaagtgtaatttacatgcactggttccagcagaagccaggcacttctcccaaactctggatttatagcacatccaacctggcttctggagtccctgctcgcttcagtggcagtggatctgggacatcttactctctcacaatcagccgaatggaggctgaagatgctgccacttattactgccagcaaaggagaagttacccgtacacgttcggaggggggaccaagctggaaataaaacgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 108 - DNA, 54 bp, artificial
  MB7 signal peptide
atgaggtggtcctggatcttcctgctgctgctgagcatcaccagcgccaacgcc

SEQ ID NO: 109 - DNA, 408 bp, artificial
  MB7-VH-122A2
atgaggtggtcctggatcttcctgctgctgctgagcatcaccagcgccaacgcccaggtccagctgcagcagtctggggctgagctggtgaggcctggggtctcagtgaagatttcctgcaagggttctggctacacattcactgattattctatgcactgggtgaagcagagtcatgcaaagagtctagagtggattggagttattagtacttactatggtgattctaactataaccagaagttcaagggcaaggccacaatgactgtagacaaatcctccaccacagcctatatggaacttgccagactgacatctgaggattctgccatctattactgtgcaagaaatggtaatttctatgttatggactactggggtcaaggaacctcagtcaccgtctcctca

SEQ ID NO: 110 - DNA, 417 bp, artificial
  MB7-VH-102E9
atgaggtggtcctggatcttcctgctgctgctgagcatcaccagcgccaacgcccagatccatttggtgcagtctggacctgacctgaagaagcctggagagacagtcaagatctcctgcaaggcttctggttataccttcacagactattcaatgcactgggtgaagcaggctccaggaaagggtttaaagtggatgggctggataaacactgagactggtgaaccaacatatgcagatgacttcaagggacggtttgccttctctttggaaagttctgccagcactgcctttttgcagatcaacaacctcaaaaatgaggacacgtctacatatttctgtactagaaatggttactacgtgggttactatgctatggactactggggtcaaggaacctcagtcaccgtctcctca

SEQ ID NO: 111 - DNA, 407 bp, artificial
  MB7-VH-104C12
atgaggtggtcctggatcttcctgctgctgctgagcatcaccagcgccaacgcccaggtccagctgcagcagtctggggctgagctggtggggcctggggtctcagtgaagatttcctgcaagggttctggctacacattcactgattattctatgcactgggtaaagcagagtcatgcaaagagtctagagtggattggagttattagtccttactatggtgatactaactacaaccagaagttcaagggcaaggccacaatgactgtagacaaatcctccagcacagcctatatggaacttgccagtctgacatctgaggattctgccatctatttctgtgcaagaaatgatgattactacaggtttgcttactggggccaagggactctggtcactgtctctgc

SEQ ID NO: 112 - DNA, 417 bp, artificial
  MB7-VH-114D11
atgaggtggtcctggatcttcctgctgctgctgagcatcaccagcgccaacgcccagatccagttggtgcagtctggacctgagctgaagaagcctggagagacagtcaagatctcctgcaaggcttctggttataccttcacagactcttcaatgcactgggtgcagcaggctccaaacaagggtttaaagtggatgggctggataaacactgagactggtgggccaacgtatgcagatgatttcaagggacggtttgccttctctttggaaacctctgccagaactgcctatttgcagatcaacaacctcaaaaatgaggacacggctacatatttctgtgctagaaatggatactacgtggggtactatgctctggactactggggtcaaggaacctcagtcaccgtctcctca

SEQ ID NO: 113 - DNA, 417 bp, artificial
  MB7-VH-104E10
atgaggtggtcctggatcttcctgctgctgctgagcatcaccagcgccaacgcccagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaacactgagactggtgagccaacatatgcagatgacttcaagggacggtttgccttctctttggaaacctctgccaccactgcctatttgcagatcaacaacttcaaaaatgaggacacggctacatatttctgtgctagaaatggttactacgtgggatattatgctatggactactggggtcaaggaacctcagtcaccgtctcctca

SEQ ID NO: 114 - DNA, 375 bp, artificial
  MB7-VL-122A2
atgaggtggtcctggatcttcctgctgctgctgagcatcaccagcgccaacgccgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggaccaagaagatattgccacttacttttgccaacagggtaatacgcttccttggacgttcggtggaggcaccaagctggaaatcaaa

SEQ ID NO: 115 - DNA, 372 bp, artificial
  MB7-VL-102E9
atgaggtggtcctggatcttcctgctgctgctgagcatcaccagcgccaacgcccaaattgttctcacccagtctccagcaatcatgtctgcatctccaggggagaaggtcaccataacctgcagtgccagctcaagtgtaatttacattcactggttccagcagaagccaggcacttctcccaaactctggatttatagcacatcctacctggcttctggagtccctgctcgcttcagtggcagtggatctgggacctcttactctctcacaatcagccgaatggaggctgaagatgctgccacttattactgccagcagaggagaagttacccgttcacgttcggaggggggaccaagctggaaataaaa

SEQ ID NO: 116 - DNA, 374 bp, artificial
  MB7-VL-104C12
atgaggtggtcctggatcttcctgctgctgctgagcatcaccagcgccaacgccgatctccagatgacacagactccatcctccctgtctgcctctctgggagacagagtcaccatcagttgcagggcaagtcaggacattaacaattatttaagctggtatcaggagaaaccagatggaacttttaaactcctgatctactacacatcaagattacactcaggagtcccatcaaggttcagtggcagtgggtctggaacagattattctctcaccgttcgcaacctggaacaggaagatattggcacttacttttgccaacagggtaaaacgcttccgtggacgttcggtggaggcaccaagctggaaatcag

SEQ ID NO: 117 - DNA, 372 bp, artificial
  MB7-VL-114D11
atgaggtggtcctggatcttcctgctgctgctgagcatcaccagcgccaacgcccaaattgttctcacccagtctccagcaatcatgtctgcatctccaggggagaaggtcaccataacctgcagtgccagctcaagtgtattttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagaagttacccgtacacgttcggaggggggaccaagctggaaataaaa

SEQ ID NO: 118 - DNA, 372 bp, artificial
  MB7-VL-104E10
atgaggtggtcctggatcttcctgctgctgctgagcatcaccagcgccaacgcccaaattgttctcacccagtctccagcaatcatgtctgcatctccaggggagaaggtcaccatgacctgcagtgccagttcaagtgtaatttacatgcactggttccagcagaagccaggcacttctcccaaactctggatttatagcacatccaacctggcttctggagtccctgctcgcttcagtggcagtggatctgggacatcttactctctcacaatcagccgaatggaggctgaagatgctgccacttattactgccagcaaaggagaagttacccgtacacgttcggaggggggaccaagctggaaataaaa

SEQ ID NO: 119 - DNA, 1398 bp, artificial
  MB7-VH-CH-122A2
atgaggtggtcctggatcttcctgctgctgctgagcatcaccagcgccaacgcccaggtccagctgcagcagtctggggctgagctggtgaggcctggggtctcagtgaagatttcctgcaagggttctggctacacattcactgattattctatgcactgggtgaagcagagtcatgcaaagagtctagagtggattggagttattagtacttactatggtgattctaactataaccagaagttcaagggcaaggccacaatgactgtagacaaatcctccaccacagcctatatggaacttgccagactgacatctgaggattctgccatctattactgtgcaagaaatggtaatttctatgttatggactactggggtcaaggaacctcagtcaccgtctcctcagcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 120 - DNA, 1407 bp, artificial
  MB7-VH-CH-102E9
atgaggtggtcctggatcttcctgctgctgctgagcatcaccagcgccaacgcccagatccatttggtgcagtctggacctgacctgaagaagcctggagagacagtcaagatctcctgcaaggcttctggttataccttcacagactattcaatgcactgggtgaagcaggctccaggaaagggtttaaagtggatgggctggataaacactgagactggtgaaccaacatatgcagatgacttcaagggacggtttgccttctctttggaaagttctgccagcactgcctttttgcagatcaacaacctcaaaaatgaggacacgtctacatatttctgtactagaaatggttactacgtgggttactatgctatggactactggggtcaaggaacctcagtcaccgtctcctcagcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 121 - DNA, 1397 bp, artificial
  MB7-VH-CH-104C12
atgaggtggtcctggatcttcctgctgctgctgagcatcaccagcgccaacgcccaggtccagctgcagcagtctggggctgagctggtggggcctggggtctcagtgaagatttcctgcaagggttctggctacacattcactgattattctatgcactgggtaaagcagagtcatgcaaagagtctagagtggattggagttattagtccttactatggtgatactaactacaaccagaagttcaagggcaaggccacaatgactgtagacaaatcctccagcacagcctatatggaacttgccagtctgacatctgaggattctgccatctatttctgtgcaagaaatgatgattactacaggtttgcttactggggccaagggactctggtcactgtctctgcgcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 122 - DNA, 1407 bp, artificial
  MB7-VH-CH-114D11
atgaggtggtcctggatcttcctgctgctgctgagcatcaccagcgccaacgcccagatccagttggtgcagtctggacctgagctgaagaagcctggagagacagtcaagatctcctgcaaggcttctggttataccttcacagactcttcaatgcactgggtgcagcaggctccaaacaagggtttaaagtggatgggctggataaacactgagactggtgggccaacgtatgcagatgatttcaagggacggtttgccttctctttggaaacctctgccagaactgcctatttgcagatcaacaacctcaaaaatgaggacacggctacatatttctgtgctagaaatggatactacgtggggtactatgctctggactactggggtcaaggaacctcagtcaccgtctcctcagcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 123 - DNA, 1407 bp, artificial
  MB7-VH-CH-104E10
atgaggtggtcctggatcttcctgctgctgctgagcatcaccagcgccaacgcccagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaacactgagactggtgagccaacatatgcagatgacttcaagggacggtttgccttctctttggaaacctctgccaccactgcctatttgcagatcaacaacttcaaaaatgaggacacggctacatatttctgtgctagaaatggttactacgtgggatattatgctatggactactggggtcaaggaacctcagtcaccgtctcctcagcctccaccaagggcccatccgtgttccccctggccccatccagcaagtctacctccggaggcacagccgccctgggctgtctggtgaaggactacttccccgagccagtgaccgtgtcctggaactccggagccctgacatccggcgtgcacaccttccccgccgtgctgcagtccagcggcctgtactctctgtcttccgtggtgaccgtgccatccagctccctgggaacccagacatacatctgcaacgtgaaccacaagcctagcaacaccaaggtggacaagaaggtggagcctaagagctgtgacaagacacacacatgccctccttgtccagcccctgagctgctgggcggcccctccgtgttcctgttcccccccaagcctaaggataccctgatgatcagcagaacccccgaggtgacctgcgtggtggtggacgtgtcccacgaggatcccgaggtgaagttcaactggtacgtggacggcgtggaggtgcacaacgctaagaccaagcccagagaggagcagtacaacagcacatacagagtggtgtctgtgctgaccgtgctgcaccaggactggctgaacgggaaggagtacaagtgcaaggtgtccaacaaggccctgcctgcccctatcgagaagaccatctctaaggctaaggggcagccccgggagccacaggtgtacaccctgccacccagccgcgacgagctgaccaagaaccaggtgtccctgacatgcctggtgaagggattctaccccagcgacatcgccgtggagtgggagagcaacggccagcccgagaacaactacaagacaacccctcccgtgctggacagcgatggatccttcttcctgtactccaagctgaccgtggacaagagcaggtggcagcagggaaacgtgttctcttgttccgtgatgcacgaggctctgcacaaccactacacccagaagtccctgagcctgtctccaggcaag

SEQ ID NO: 124 - DNA, 696 bp, artificial
  MB7-VL-CL-122A2
atgaggtggtcctggatcttcctgctgctgctgagcatcaccagcgccaacgccgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggaccaagaagatattgccacttacttttgccaacagggtaatacgcttccttggacgttcggtggaggcaccaagctggaaatcaaacgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 125 - DNA, 693 bp, artificial
  MB7-VL-CL-102E9
atgaggtggtcctggatcttcctgctgctgctgagcatcaccagcgccaacgcccaaattgttctcacccagtctccagcaatcatgtctgcatctccaggggagaaggtcaccataacctgcagtgccagctcaagtgtaatttacattcactggttccagcagaagccaggcacttctcccaaactctggatttatagcacatcctacctggcttctggagtccctgctcgcttcagtggcagtggatctgggacctcttactctctcacaatcagccgaatggaggctgaagatgctgccacttattactgccagcagaggagaagttacccgttcacgttcggaggggggaccaagctggaaataaaacgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 126 - DNA, 695 bp, artificial
  MB7-VL-CL-104C12
atgaggtggtcctggatcttcctgctgctgctgagcatcaccagcgccaacgccgatctccagatgacacagactccatcctccctgtctgcctctctgggagacagagtcaccatcagttgcagggcaagtcaggacattaacaattatttaagctggtatcaggagaaaccagatggaacttttaaactcctgatctactacacatcaagattacactcaggagtcccatcaaggttcagtggcagtgggtctggaacagattattctctcaccgttcgcaacctggaacaggaagatattggcacttacttttgccaacagggtaaaacgcttccgtggacgttcggtggaggcaccaagctggaaatcagcgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 127 - DNA, 693 bp, artificial
  MB7-VL-CL-114D11
atgaggtggtcctggatcttcctgctgctgctgagcatcaccagcgccaacgcccaaattgttctcacccagtctccagcaatcatgtctgcatctccaggggagaaggtcaccataacctgcagtgccagctcaagtgtattttacatgcactggttccagcagaagccaggcacttctcccaaactctggatttatagcacatccaacctggcttctggagtccctgctcgcttcagtggcagtggatctgggacctcttactctctcacaatcagccgaatggaggctgaagatgctgccacttattactgccagcaaaggagaagttacccgtacacgttcggaggggggaccaagctggaaataaaacgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 128 - DNA, 693 bp, artificial
  MB7-VL-CL-104E10
atgaggtggtcctggatcttcctgctgctgctgagcatcaccagcgccaacgcccaaattgttctcacccagtctccagcaatcatgtctgcatctccaggggagaaggtcaccatgacctgcagtgccagttcaagtgtaatttacatgcactggttccagcagaagccaggcacttctcccaaactctggatttatagcacatccaacctggcttctggagtccctgctcgcttcagtggcagtggatctgggacatcttactctctcacaatcagccgaatggaggctgaagatgctgccacttattactgccagcaaaggagaagttacccgtacacgttcggaggggggaccaagctggaaataaaacgaactgtggctgcaccaagtgtcttcatctttcctccgagtgatgagcagctgaagagcgggacagcttctgtggtgtgtctgctgaataacttctacccaagagaagcaaaggtccagtggaaggtggacaacgccctgcagtctggcaactcacaggagtctgtcactgagcaggattccaaggacagcacttacagcctgtccagcaccctcactctgtccaaagccgactacgaaaagcataaggtgtatgcttgtgaggtgacccaccagggactgagcagccctgtgacgaagtccttcaaccggggcgagtgc

SEQ ID NO: 129 - DNA, 1313 bp, Homo sapiens
  misc_feature: CD303 humain (AF293615.1)
cagtgattctcgtgcctcagcctcctgagtagccgaaattacagacgtgtgccaccatgcttggctaattttttggatttttagtagagatggggtttcactatgttggccaggctagtcttgaactcctggcctgaagcaatccgcccacctcagcctcccaaagtgctgagattataggcacgagccactacacctggccacaaaattctttaaagaagccaatcccatcctccctcaagagccaaggggccacctcaccctcttgttacagcagatcctgcctcccacagtcaccctgctcccaagtgcaacctctgtctgaccctgcatggtgtgcggtgccctcctgcctcaggccgcgaagaaggatctaagggcttggcttgtttgaaagaaccacaccccgaaagtaacatctttggagaaagtgatacaagagcttctgcacccacctgatagaggaagtccaaagggtgtgcgcacacacaatggtgcctgaagaagagcctcaagaccgagagaaaggactctggtggttccagttgaaggtctggtccatggcagtcgtatccatcttgctcctcagtgtctgtttcactgtgagttctgtggtgcctcacaattttatgtatagcaaaactgtcaagaggctgtccaagttacgagagtatcaacagtatcatccaagcctgacctgcgtcatggaaggaaaggacatagaagattggagctgctgcccaaccccttggacttcatttcagtctagttgctactttatttctactgggatgcaatcttggactaagagtcaaaagaactgttctgtgatgggggctgatctggtggtgatcaacaccagggaagaacaggatttcatcattcagaatctgaaaagaaattcttcttattttctggggctgtcagatccagggggtcggcgacattggcaatgggttgaccagacaccatacaatgaaaatgtcacattctggcactcaggtgaacccaataaccttgatgagcgttgtgcgataataaatttccgttcttcagaagaatggggctggaatgacattcactgtcatgtacctcagaagtcaatttgcaagatgaagaagatctacatataaatgaaatattctccctggaaatgtgtttgggttggcatccaccgttgtagaaagctaaattgattttttaatttatgtgtaagttttgtacaaggaatgcccctaaaatgtttcagcaggctgtcacctattacacttatgatataatccaaaaaaaaaaaaaaaaaaaaaaaaaaaaaa

SEQ ID NO: 130 - protein, 213 aa, Homo sapiens
  SITE (1)..(213): CD303 humain (AAL37036.1)
MVPEEEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI